(19) United States
(12) Patent Application Publication
(10) Pub. No.: US 2004/0014174 A1
(43) Pub. Date: Jan. 22, 2004

(54) Expression of polypeptides in chloroplasts, and compositions and methods for expressing same

(76) Inventors: Stephen P. Mayfield, Cardiff, CA (US); Scott Franklin, Cardiff, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/422,628

(22) Filed: Apr. 23, 2003

Related U.S. Application Data
(60) Provisional application No. 60/434,957, filed on Dec. 19, 2002.
(60) Provisional application No. 60/375,129, filed on Apr. 23, 2002.

(51) Int. Cl. C12P 21/02; C07H 21/04; C12N 5/04; C07K 16/00

(52) U.S. Cl. 435/69.1; 435/320.1; 435/419; 530/387.1; 536/23.5

(57) ABSTRACT

Methods of producing one or more polypeptides in a plant chloroplast, including methods of producing polypeptides that specifically associate in a plant chloroplast to generate a functional protein complex, are provided. An isolated polynucleotide that includes (or encodes) a first ribosome binding sequence (RBS) operatively linked to a second RBS, such that the first RBS directs translation of a polypeptide in a prokaryote and the second RBS directs translation of the polypeptide in a chloroplast, also is provided, as is a vector containing such a polynucleotide, particularly a chloroplast vector and a chloroplast/prokaryote shuttle vector. Also provided is a synthetic polynucleotide, which is chloroplast codon biased. A plant cell that is genetically modified to contain a polynucleotide or vector as described above, as well as transgenic plants containing or derived from such a genetically modified cell, are provide. Polypeptides encoded by a synthetic polynucleotide as described also are provided.

This application claims the benefit of priority under 35 U.S.C. §119 of U.S. Serial No. 60/375,129, filed Apr. 23, 2002, and U.S. Serial No. 60/434,957, filed Dec. 19, 2002, the entire content of each of which is incorporated herein by reference.

This invention was made in part with government support under Grant No. GM54659 awarded by the National Institutes of Health, Grant No. DE-FG03-93ER20116 awarded by the Department of Energy, and Grant No NA06RG00142 awarded by the California Sea Grant college program of the National Oceanic and Atmospheric Administration. The government may have certain rights in this invention.

### BACKGROUND OF THE INVENTION

1. Field of the Invention

The present invention relates generally to compositions and methods for expressing polypeptides in plant cell chloroplasts, and more specifically to chloroplast codon biased polynucleotides encoding heterologous polypeptides, to expression vectors that allow robust expression of heterologous polypeptides in bacteria and in chloroplasts, including, for example, of protein complexes such as antibodies and antibody chimeras that are formed by a specific association of polypeptide subunits.

2. Background Information

Molecular biology and genetic engineering hold promise for the production of large quantities of biologically active compounds that can be used as supplements for healthy individuals or as therapeutic agents for treating individuals having a pathologic disorder. For example, growth hormone has been produced using genetic engineering methods and the recombinant growth hormone has been used to treat individuals suffering from growth stunting disorders. Similarly, monoclonal antibodies having desirable specificity characteristics are finding use as therapeutic agents for various disorders, including cancers such as lymphomas and breast cancer.

A primary advantage of using genetic engineering techniques for producing therapeutic biological agents is that the methods allow for the generation of large amounts of a desired protein. In many cases, the only other way to obtain sufficient quantities of the biological material, for example, for use as a therapeutic agent, is by purifying the naturally occurring biological material from cells of an organism that produce the agent. Thus, prior to the advent of genetic engineering, growth hormone could only be obtained by isolating it from the pituitary gland of animals such as cattle. Insulin is another example of a biological agent that, prior to genetic engineering, was available in a sufficient amount and in a biologically active form only by isolating it from the pancreas of animals such as pigs.

Although genetic engineering provides a means to produce large amounts of a biological material, particularly proteins and nucleic acids, there are limitations to currently available methods. For example, human proteins can be expressed in large quantities in bacterial cells. However, bacteria do not provide an environment suitable to the assembly of more complex proteins such as antibodies, in which four polypeptide chains, for example, associate to form the biologically active protein. Thus, even where bacteria can be used to produce the biological material, additional steps such as denaturing and refolding the protein under defined conditions may be required to obtain biologically active material.

Recombinant proteins also can be produced in eukaryotic cells, including, for example, insect cells and mammalian cells, which may provide the necessary environment and accessory factors required to process an expressed protein into a biologically active agent. For example, antibodies contain a heavy chain and a light chain that form a dimer with each other, and further associates with a second heavy chain and light chain dimer to form an active antibody. Such a process can occur in eukaryotic cells such as mammalian cells. However, eukaryotic cells also can modify a protein, for example, by glycosylating the protein such that it contains sugar groups at specific positions. While such post-translational modifications can result in advantageous characteristics, they also can provide disadvantages that limit the usefulness of the recombinant protein. For example, glycosyl groups can be strongly antigenic and, upon administration to an individual, can result in the stimulation of an immune response that can inactivate the recombinant protein and, in some cases, can produce deleterious effects that cause more harm to the individual than the condition for which the recombinant protein originally was administered.

Generally, a polynucleotide encoding a polypeptide that is to be produced using recombinant DNA methods is contained in a vector, which is a nucleic acid molecule that facilitates manipulation of the polynucleotide. Vectors can be used for introducing a polynucleotide of interest in prokaryotic cells such as bacteria or into eukaryotic cells such as mammalian cells. Depending on the host cell in which the vector is to be contained, the vector also contains regulatory elements that allow, for example, amplification of the vector in the host cell. In addition, vectors have been designed that allow passage in both prokaryotic and eukaryotic cells. Such shuttle vectors can be useful because they allow, for example, generation of large amounts of the vector (and polynucleotide contained therein) in bacteria, then the vectors can be transferred to mammalian cells such that the encoded polypeptide can be produced under conditions that allow for proper assembly of a biologically active protein.

Although such shuttle vectors provide advantages over vectors that are specific for one or a few specific cell types, they do not obviate the potential problems that may be caused by post-translational modifications such as glycosylation, which can occur in eukaryotic cells. Thus, a need exists for methods to conveniently produce proteins that are biologically active, but do not, for example, have undesirable characteristics such as a strong antigenicity when administered to an individual such as a human. The present invention satisfies this need and provides additional advantages.

### SUMMARY OF THE INVENTION

The present invention is based, in part, on a determination that heterologous polypeptides can be expressed robustly in plants by modifying the nucleotide sequence encoding the polypeptide such that it reflects chloroplast codon usage. Accordingly, the present invention relates to a synthetic polynucleotide, which includes at least a first nucleotide sequence encoding at least a first polypeptide, wherein at least one codon in the first nucleotide sequence is biased to reflect chloroplast codon usage. In one embodiment, each codon in the first nucleotide sequence is biased to reflect chloroplast codon usage.

The synthetic polynucleotide can contain a single nucleotide sequence encoding a single polypeptide, or can further include at least a second nucleotide sequence encoding a second polypeptide, wherein one or more of the codons of the second nucleotide sequence also can be biased to reflect chloroplast codon usage. Where the synthetic polynucleotide encodes two or more polypeptides, the encoding nucleotide sequences can be operatively linked such that a single polynucleotide is transcribed therefrom, and the encoded polypeptides can be expressed separately or can be further operatively linked such that a fusion protein comprising the first polypeptide and the second polypeptide can be expressed. In one embodiment, a first and second nucleotide sequence are operatively linked via a third nucleotide sequence, which, for example, can encode a linker peptide. As such, a fusion protein comprising the first polypeptide linked via the linker peptide to the second polypeptide can be expressed from the synthetic polynucleotide.

The polypeptide(s) encoded by a synthetic polynucleotide of the invention can be any polypeptide of interest, and generally is a polypeptide that is not normally expressed in a plastid, particularly a chloroplast. For example, the encoded polypeptide(s) can be an one or more chains of an immunoglobulin (Ig) family member, e.g., Ig variable region, an Ig constant region, an Ig heavy chain, an Ig light chain, or a combination thereof, or a T cell receptor (TCR) a chain, TCR p chain, or combination thereof, or any soluble receptor such as soluble forms of a T cell receptor or fusions of such receptors with, for example, an IG heavy chain. In one embodiment, the synthetic polynucleotide encodes an Ig family member fusion protein, for example, a single chain antibody comprising a complete heavy chain operatively linked to a light chain variable region. Such a fusion protein is exemplified herein by a single chain anti-herpes simplex virus (HSV) antibody having an amino acid sequence as set forth in SEQ ID NO: 16, which can be encoded by the synthetic polynucleotide having a nucleotide sequence as forth in SEQ ID NO: 15, which is biased to reflect chloroplast codon usage. In another example, a fusion protein encoded by a synthetic polynucleotide that is biased to reflect chloroplast codon usage is exemplified by the single chain anti-HSV Fv fragment having an amino SCRIP1510-25 acid sequence as set forth in SEQ ID NO:43, which is encoded by SEQ ID NO:42. In still another example, an fusion protein encoded by a synthetic polynucleotide that is biased to reflect chloroplast codon usage is exemplified by the HSV8-lsc (large single chain) antibody having an amino acid sequence as set forth in SEQ ID NO:48, which is encoded by SEQ ID NO:48.

A polypeptide encoded by a synthetic polynucleotide of the invention also can be a reporter polypeptide, for example, a luciferase polypeptide. Such a luciferase reporter polypeptide is exemplified herein by the luciferase fusion protein comprising the bacterial luciferase A subunit operatively linked via a linker peptide to the bacterial luciferase B subunit, the fusion protein having an amino acid sequence as set forth in SEQ ID NO:46, which can be encoded by the synthetic polynucleotide having a nucleotide sequence as set forth in SEQ ID NO:45, which is biased to reflect chloroplast codon usage. Accordingly, a luciferase fusion polypeptide having an amino acid sequence as set forth in SEQ ID NO:46 is provided. A synthetic chloroplast codon biased polynucleotide encoding a reporter polypeptide, such as the exemplified polynucleotide (SEQ ID NO:45) encoding a fusion bacterial luxAB polypeptide (SEQ ID NO:46) can be useful, for example, as a tool to identify chloroplast promoters, 5′ untranslated regions (5′ UTRs), 3′ UTR, protease deficient strains, and the like, thus providing a means to obtain further improved expression of a heterologous polypeptide in a chloroplast.

The present invention also relates to a method of producing a heterologous polypeptide in a plastid by introducing a synthetic polynucleotide that includes at least a first nucleotide sequence encoding at least a first polypeptide, wherein at least one codon in the first nucleotide sequence is biased to reflect chloroplast codon usage, into the plastid under conditions that allow expression of the at least first polypeptide in the plastid. The synthetic polynucleotide can be operatively linked to a nucleic acid sequence encoding at least one ribosome binding sequence (RBS), particularly an RBS that can direct translation of the polypeptide in a plastid.

The synthetic polynucleotide used according to a method of the invention can be any synthetic polynucleotide comprising at least a first nucleotide sequence containing at least one codon that is biased to reflect chloroplast codon usage. As such, the synthetic polynucleotide can further include at least a second nucleotide sequence encoding a second polypeptide, wherein the first nucleotide sequence can, but need not, be operatively linked to the second nucleotide sequence, and wherein the second polypeptide can, but need not, be heterologous to the chloroplast. Where the synthetic polynucleotide encodes two (or more) polypeptides, the encoded polypeptides can be expressed as separate and distinct polypeptides, or as a fusion protein comprising the first and second (or more) polypeptides.

In one embodiment, a fusion protein expressed from a synthetic polynucleotide according to a method of the invention comprises a first polypeptide linked via a linker peptide to a second polypeptide. Such a method is exemplified herein by expressing a single chain antibody comprising an IgA heavy chain linked to a light chain variable region, the fusion protein having an amino acid sequence as set forth in SEQ ID NO: 16, and encoded by a nucleotide sequence as set forth in SEQ ID NO: 15, which is biased with respect to chloroplast codon usage, wherein the expressed single chain antibody maintains antigen binding specificity (see, also, single chain anti-HSV Fv fragment having an amino acid sequence as set forth in SEQ ID NO:43 (encoded by SEQ ID NO:42), and HSV8-lsc antibody having an amino acid sequence as set forth in SEQ ID NO:48 (encoded by SEQ ID NO:48).

A method of the invention is further exemplified herein by expressing a reporter polypeptide, particularly a luciferase fusion protein comprising the luciferase A subunit operatively linked to the luciferase B subunit, the fusion protein having an amino acid sequence as set forth in SEQ ID NO:46, and encoded by a nucleotide sequence as set forth in SEQ ID NO:45, wherein expression of the heterologous luciferase in chloroplasts is detectable in vivo or in vitro.

A method of the invention can be practiced in any plastid, including in plant chloroplasts. The plant containing the chloroplasts can be any plant that naturally contains chloroplasts, including alga (microalga or macroalga) and higher plants. The method can further include a step of isolating the expressed heterologous polypeptide from plant cells (or isolated chloroplasts) containing the polypeptide. Accordingly, the invention provides a heterologous polypeptide produced by the method of the invention.

The present invention further relates to a method of detecting a plant cell that contains a plastid. Such a method can be performed, for example, by introducing a synthetic polynucleotide of the invention, wherein the polynucleotide encodes a reporter polypeptide, into a plastid, e.g., a chloroplast, of the plant cell under conditions that allow expression of the reporter polypeptide in the chloroplast, and detecting expression of the reporter polypeptide. The reporter polypeptide can be any polypeptide as desired, and is exemplified herein by expressing a luciferase fusion protein having an amino acid sequence as set forth in SEQ ID NO:46.

The present invention also relates to a method of producing a polypeptide in a plastid. Such a method can be performed, for example, by introducing at least a first recombinant nucleic acid molecule into the plastid, wherein the first recombinant nucleic acid molecule includes a first nucleotide sequence encoding at least one ribosome binding sequence (RBS) operatively linked to at least one heterologous polynucleotide encoding at least one polypeptide, and wherein the RBS can direct translation of the polypeptide in a plastid, under conditions that allow expression of the at least one polypeptide, thereby producing the polypeptide in the plastid. The plastic can be any plastid, including, for example, a chloroplast.

According to the present method, one or more codons of the first polynucleotide can be biased to reflect chloroplast codon usage. In one embodiment, the encoded polypeptide is an antibody, or a subunit of an antibody. In another embodiment, the first polynucleotide encodes a first polypeptide and a second polypeptide, for example, a first polypeptide comprising an Ig heavy chain or a variable region thereof, and a second polypeptide comprises an Ig light chain or a variable region thereof. Such an antibody expressed according to a method of the invention is exemplified by an anti-tetanus toxin antibody having an amino acid sequence as set forth in SEQ ID NO: 14, which is encoded by the nucleotide sequences as set forth in SEQ ID NO: 13. In still another embodiment, the first polynucleotide is biased for chloroplast codon usage. Such antibodies expressed according to a method of the invention are exemplified by an anti-HSV antibody having an amino acid sequence as set forth in each of SEQ ID NO: 16, SEQ ID NO:43, and SEQ ID NO:48, such antibodies being encoded, for example, by the nucleotide sequences as set forth in SEQ ID NO: 15, SEQ ID NO:42, SEQ ID NO:47, respectively.

In another embodiment, the first polynucleotide encodes a first polypeptide and at least a second polypeptide, wherein the first and second (or more) polypeptides can, but need not, be subunits of a protein complex, for example, a heterodimer, heterotrimer, etc. In still another embodiment, the method can further include introducing at least a second recombinant nucleic acid molecule into the plastid. Such a second recombinant nucleic acid molecule can include a first nucleotide sequence encoding at least a first RBS operatively linked to at least a second heterologous polypeptide encoding at least a second polypeptide, wherein the first RBS can direct translation of the polypeptide in a plastid, particularly a chloroplast. Preferably, the first recombinant nucleic acid molecule and the second recombinant nucleic acid molecule are co-expressed in the plastid.

According to a method of the invention, the first recombinant nucleic acid molecule can be contained in a vector. In one embodiment, the vector is a chloroplast vector, which comprises a nucleotide sequence of chloroplast genomic DNA that can undergo homologous recombination with chloroplast genomic DNA, and the vector containing the first recombinant nucleic acid molecule is introduced into a chloroplast. Such a vector can further contain a prokaryote origin of replication.

A method of the invention can further include isolating the polypeptide from the plastid. Accordingly, the invention provides an isolated polypeptide obtained by such a method, for example, an isolated antibody that is expressed in and heterologous with respect to a chloroplast.

The present invention further relates to method of producing one or more polypeptides in a plant chloroplast, including methods of producing polypeptides that specifically associate to form a protein complex. As such, a method of the invention provides a means to produce functional protein complexes, for example, a bivalent antibody comprising a first heavy and light chain associated with a second heavy and light chain. A method of the invention can be performed, for example, by introducing a first recombinant nucleic acid molecule into a chloroplast, which includes a first polynucleotide encoding at least one polypeptide; operatively linked to a second polynucleotide, which comprises a nucleotide sequence encoding a first ribosome binding sequence (RBS) operatively linked to a nucleotide sequence encoding a second RBS, wherein the first RBS can direct translation of the polypeptide in a prokaryote and the second RBS can direct translation of the polypeptide in a chloroplast, under conditions that allow expression of the at least one polypeptide, thereby producing the polypeptide in the chloroplast. The methods of the invention can be performed using any plant (or plant cell) that contains chloroplasts, including unicellular plants and algae and multicellular plants and algae.

In one embodiment, the first polynucleotide used in a method of the invention encodes a first polypeptide and at least a second polypeptide, for example, a first polypeptide and a second polypeptide; or a first polypeptide, a second polypeptide, and a third polypeptide; etc., any or all of which can be the same or different. In another embodiment, one or more codons of the first polynucleotide are biased to reflect chloroplast codon usage.

As disclosed herein, polypeptides expressed in plant chloroplasts such as chloroplasts of the microalga Chlamydomonas reinhardtii assemble properly, and can associate with one or more other expressed polypeptides in the chloroplast to form a functional protein complex. Accordingly, in still another embodiment, a first polynucleotide useful in a method of the invention can encode one or more polypeptide subunits that can associate to form a functional protein complex. The protein complex can be a dimer, trimer, tetramer, or the like, and the subunits can be the same or different or a combination thereof. For example, where the protein complex is a dimer, it can be a homodimer or a heterodimer. Where the protein complex is a trimer, it can be a homotrimer, a heterotrimer, or a trimer consisting of two identical polypeptides and one different polypeptide.

A method of the invention is particularly useful for producing functional protein complexes such as antibodies, which generally occur naturally as a complex containing two heavy chains and two light chains, cell surface receptors such as T cell receptors, growth factor receptors, hormone receptors, G-protein coupled receptors, which can associate with a G-protein, and the like. An advantage of using a method of the invention to produce proteins such as antibodies in a chloroplast is that the polypeptides are not glycosylated following expression in chloroplasts and, therefore, have a greatly reduced antigenicity as compared to antibodies raised in an animal or expressed in the cytoplasm of a eukaryotic cell. As disclosed herein, a method of producing a functional protein complex in a chloroplast can be performed using a first recombinant nucleic acid molecule, as defined, wherein the first polynucleotide encodes the two or more subunits of the complex; or using a first recombinant nucleic acid molecule, as defined, which encodes one polypeptide subunit of the complex, and a second recombinant nucleic acid molecule, which has the same defined characteristics as the first recombinant nucleic acid molecule, and which encodes an additional polypeptide subunit of the protein complex.

Accordingly, a method of the invention can be practiced using a first recombinant nucleic acid molecule, wherein the first polynucleotide encodes a first polypeptide, which is an immunoglobulin heavy chain (H) or a variable region thereof, and a second polypeptide, which is an immunoglobulin light chain (L) or a variable region thereof. If desired, a nucleotide sequence encoding an internal ribosome entry site can be positioned between the nucleotide sequences encoding the H and L chains such that expression of the second (downstream) encoded polypeptide is facilitated. Upon translation of the encoded H and L chains in the chloroplast, a H chain can associate with a L chain to form a monovalent antibody (i.e., an H:L complex), and two H:L complexes can further associate to produce a bivalent antibody.

A method of the invention also can be practiced by introducing into a plant chloroplast a first recombinant nucleic acid molecule, wherein the first polynucleotide encodes, for example, a H chain or a variable region thereof, and further introducing into the chloroplast a second recombinant nucleic acid molecule, which comprises a first polynucleotide encoding a L chain or a variable region thereof, operatively linked to a second polynucleotide that includes a nucleotide sequence encoding a first RBS operatively linked to a nucleotide sequence encoding a second RBS, wherein the first RBS can direct translation of the polypeptide in a prokaryote and the second RBS can direct translation of the polypeptide in a chloroplast, under conditions such that the encoded polypeptides are substantially co-expressed in the chloroplasts, wherein the heavy chains (H) and light chains (L) can associate to form an H:L complex, and wherein H:L complexes can further associate to produce a bivalent antibody.

In practicing a method of the invention, the first recombinant nucleic acid molecule can be contained in a vector. Furthermore, where the method is performed using a second (or more) other recombinant nucleic acid molecules, the second recombinant nucleic acid molecule also can be contained in a vector, which can, but need not, be the same vector as that containing the first recombinant nucleic acid molecule. Alternatively, a plant cell can be genetically modified such that chloroplasts in the plant contain a stably integrated recombinant nucleic acid molecule encoding a subunit of a protein complex, and the method of the invention can comprise introducing, for example, a vector comprising a second recombinant nucleic acid molecule, which encodes one or more other subunits of the protein complex, into chloroplasts of the plant such that, upon expression, a functional protein complex is produced.

A vector useful in a method of the invention can be any vector useful for introducing a polynucleotide into a chloroplast. In particular, the vector can include a nucleotide sequence of chloroplast genomic DNA sufficient to undergo homologous recombination with chloroplast genomic DNA. Such a chloroplast vector can contain any additional nucleotide sequence that facilitates use or manipulation of the vector, for example, one or more transcriptional regulatory elements, or selectable markers, or cloning sites, or the like, including combinations thereof. In one embodiment, the vector, which can be a chloroplast vector, includes a transcriptional promoter and a 5′-untranslated region (5′UTR) of a plant chloroplast gene, which further contains, or can be modified to contain, a first RBS operatively linked to a second RBS, as defined herein. In another embodiment, the vector, which can be a chloroplast vector, includes a prokaryote origin of replication (ori), for example, an E. coli ori, thus providing a shuttle vector that can be passaged and manipulated in a prokaryote host cell as well as in a chloroplast. A shuttle vector of the invention can contain any polynucleotide of interest, including a synthetic chloroplast codon biased polynucleotide, for example, a synthetic polynucleotide such as SEQ ID NO:45, which encodes a bacterial luxAB fusion protein (SEQ ID NO:46). Such a shuttle vector expressing SEQ ID NO:46 provides the advantage that regulatory elements or other sequences of interest can be examined for expression in bacteria, then vectors containing those elements have desirable expression characteristics can be shuttled, with the same or other synthetic or other polynucleotide operatively linked thereto, to chloroplasts, wherein improved expression of an encoded heterologous polypeptide can be obtained.

A method of the invention can further include a step of isolating an expressed polypeptide or protein complex from the chloroplast. Accordingly, the present invention also provides an isolated polypeptide or protein complex produced by a method as disclosed herein. For example, the present invention provides isolated antibodies, which are expressed in and obtained from a plant chloroplast. An advantage of an isolated antibody of the invention is that the polypeptide components of the antibody are not glycosylated and, therefore, the antibody has reduced antigenicity when administered to a individual. Furthermore, such an antibody of the invention can have reduced effector activities characteristic of a naturally occurring antibody, for example, complement fixation activity, thus providing antibodies that can be useful for diagnostic purposes in an individual.

The present invention also relates to an isolated ribonucleotide sequence that includes a first ribosome binding sequence (RBS) operatively linked to a second RBS, wherein the first RBS and second RBS are spaced apart by about 5 to 25 nucleotides, and wherein, when the ribonucleotide sequence is operatively linked to a polynucleotide encoding a polypeptide, the first RBS directs translation of the polypeptide in a prokaryote and the second RBS directs translation of the polypeptide in a chloroplast. An isolated ribonucleotide sequence of the invention, which generally is about 11 to 50 ribonucleotides in length, and can be about 15 to 40 ribonucleotides in length, or about 20 to 30 ribonucleotides, can be a discrete unit, or can be operatively linked to a heterologous RNA molecule.

The first RBS and second RBS, which are operatively linked in a ribonucleotide sequence of the invention, generally are spaced apart by about 5 to 25 nucleotides, and usually by about 10 to 20 nucleotides, for example, by about 15 nucleotides. Each of the first RBS and the second RBS independently can consist of about 3 to 9 nucleotides, usually about 4 to 7 nucleotides, and can have any sequence characteristic of a Shine-Delgarno (SD) sequence, for example, a sequence comprising 5′-GGAG-3′, which is complementary to a portion of a 16S rRNA anti-SD sequence. The second RBS, which directs translation in a chloroplast, can be contained within a 5′ UTR of a chloroplast gene, which can be a chloroplast gene encoding a soluble chloroplast protein or a membrane bound chloroplast protein, wherein the 5′ UTR can further include transcriptional regulatory elements, including a promoter.

A ribonucleotide sequence of the invention can further include an initiator AUG codon operatively linked to the first and second RBS. Such an initiator AUG codon can further include adjacent nucleotides of a Kozak sequence, for example, ACCAUGG, which can facilitate translation of a polypeptide in a cell. A ribonucleotide sequence of the invention also can be operatively linked to a polyribonucleotide encoding a polypeptide, which can contain an endogenous initiator AUG codon or can be modified to contain an initiator AUG codon, or can lack an initiator AUG codon, which can be a component of the ribonucleotide sequence of the invention.

An isolated ribonucleotide sequence of the invention can be chemically synthesized, or can be generated using an enzymatic method, for example, from a deoxyribonucleic acid (DNA) or ribonucleic acid (RNA) template using a DNA dependent RNA polymerase or an RNA dependent RNA polymerase, respectively. Such a DNA template can be chemically synthesized, or can be isolated from a naturally occurring DNA molecule, or can be based on naturally occurring DNA sequence that is modified to have the required characteristics, for example, a DNA sequence of a prokaryote gene that has nucleotide sequence encoding an RBS positioned about 5 to 15 nucleotides upstream an initiator ATG codon, and that is further modified to contain a second RBS, which is upstream of and spaced apart from the first RBS such that the second RBS can direct translation in a chloroplast.

Accordingly, the present invention also relates to a polynucleotide encoding a first RBS operatively linked to a second RBS, as defined herein. The polynucleotide can be DNA or RNA, and can be single stranded or double stranded. A polynucleotide of the invention can include an initiator ATG codon operatively linked to the nucleotide sequence encoding the first RBS and second RBS. In addition, a polynucleotide of the invention can include a cloning site that is positioned to allow operative linkage of an expressible polynucleotide, which can encode a polypeptide, to the first RBS and second RBS, such that the polypeptide can be expressed in a chloroplast or in a prokaryote host cell. The cloning site can be any nucleotide sequence that facilitates insertion or linkage of the expressible polynucleotide to the first and second RBS such that translation of an encoded polypeptide can be initiated from the first RBS and the second RBS under suitable conditions, for example, one or more restriction endonuclease recognition sites or recombinase recognition sites or a combination thereof.

A polynucleotide encoding a first and second RBS, as defined herein, can be operatively linked to an expressible polynucleotide, which can encode at least one polypeptide, including a peptide or peptide portion of a polypeptide. As such, the expressible polynucleotide can encode a first polypeptide and one or more additional polypeptides, which can be the same or different. For example, the expressible polynucleotide can encode a first polypeptide and a second polypeptide, which are different from each other. Furthermore, such a first and second polypeptide can be expressed as a fusion protein, or can be expressed as separate polypeptides, in which case a nucleotide sequence encoding an internal ribosome entry site can, but need not, be operatively linked between the coding sequence of the first polypeptide and the coding sequence of the second polypeptide, thus facilitating translation of the second polypeptide.

A polynucleotide of the invention also can be flanked by a first cloning site and a second cloning site, thus providing a cassette that readily can be inserted into or linked to a second polynucleotide. Such flanking first and second cloning sites can be the same or different, and one or both independently can be one of a plurality of cloning sites, i.e., a multiple cloning site.

In one embodiment, a polynucleotide of the invention contains, in operative linkage and in a 5′ to 3′ orientation, a nucleotide sequence encoding the second RBS, a nucleotide sequence encoding the first RBS, and an initiator ATG; and/or a nucleotide sequence complementary to such a polynucleotide. In another embodiment, a polynucleotide of the invention contains, in operative linkage and in a 5′ to 3′ orientation, a nucleotide sequence encoding the second RBS, a nucleotide sequence encoding the first RBS, an initiator ATG, and at least one cloning site; and/or a nucleotide sequence complementary to such a polynucleotide. In still another embodiment, a polynucleotide of invention contains, in operative linkage and in a 5′ to 3′ orientation, a nucleotide sequence encoding the second RBS, a nucleotide sequence encoding the first RBS, and at least one cloning site positioned about 3 to 10 nucleotides 3′ of the nucleotide sequence encoding first RBS; and/or a nucleotide sequence complementary to such a polynucleotide.

The present invention also relates to a vector, which includes a polynucleotide encoding an operatively linked first RBS and second RBS as disclosed herein, and a nucleotide sequence of chloroplast genomic deoxyribonucleic acid (DNA), which can undergo homologous recombination with chloroplast genomic DNA. Such a nucleotide sequence of chloroplast genomic DNA generally, though not necessarily, is a silent nucleotide sequence, which does not encode a chloroplast gene, and is of a sufficient length such that the vector can undergo homologous recombination with a corresponding nucleotide sequence in the chloroplast genome.

A vector of the invention also can contain one or more additional nucleotide sequences that confer desirable characteristics on the vector, including, for example, sequences that facilitate manipulation of the vector. As such, the vector can contain, for example, one or more cloning sites, for example, a cloning site, which can be a multiple cloning site, positioned such that a heterologous polynucleotide can be inserted into the vector and operatively linked to the first RBS and second RBS. The vector also can contain a prokaryote origin of replication (ori), for example, an E. coli ori or a cosmid ori, thus providing a shuttle vector, which can be passaged in a prokaryote host cell or in a plant chloroplast, as desired. Accordingly, in one embodiment, a chloroplast/prokaryote shuttle vector is provided, wherein the shuttle vector includes 1) a nucleotide sequence of chloroplast genomic DNA, which can undergo homologous recombination with chloroplast genomic DNA; 2) a prokaryotic origin; 3) a first RBS operatively linked to a second RBS, wherein the first (or second) RBS can direct translation of an operatively linked expressible polynucleotide in a chloroplast, and the second (or first) RBS can direct translation of the operatively linked expressible polynucleotide in a prokaryote; and 4) an operatively linked expressible polynucleotide, or a cloning site positioned such that a heterologous polynucleotide can be inserted into and operatively linked to the first RBS and second RBS.

A vector of the invention can be a circularized vector, or can be a linear vector, which has a first end and a second end. A linear vector of the invention can have one or more cloning sites at one or both ends, thus providing a means to circularize the vector or to link the vector to a second polynucleotide, which can be a second vector that is the same as or different from the vector of the invention. The cloning site can include a restriction endonuclease recognition site (or a cleavage product thereof), a recombinase site, or a combination of such sites.

The vector can further contain one or more expression control elements, for example, transcriptional regulatory elements, additional translational elements, and the like. In one embodiment, the vector contains an initiator ATG codon operatively linked to the sequence encoding the first RBS and second RBS, such that a polynucleotide encoding a polypeptide can be operatively linked adjacent to ATG codon and, upon transcription, can comprise an RNA that can be translated in a prokaryote and in a chloroplast. Accordingly, the vector also can contain a cloning site that is positioned to allow operative linkage of at least one heterologous polynucleotide to such an ATG codon. A vector of the invention also can contain a nucleotide sequence encoding a first polypeptide operatively linked to the first RBS and second RBS, wherein the encoding nucleotide sequence is modified to contain one or more cloning sites, including, for example, upstream of and near the ATG codon, downstream of and near the ATG codon, and/or at or near the C-terminus of the encoded polypeptide. Such a vector provides a convenient means to insert a nucleotide sequence encoding a second polypeptide therein, either by substitution of the nucleotide sequence encoding the first polypeptide, or in operative linkage near the N-terminus or C-terminus of the encoded polypeptide such that a fusion protein comprising the first and second polypeptide can be expressed.

The present invention also relates to a cell, which contains a polynucleotide of the invention or a vector of the invention. The cell, which can be a host cell for a vector of the invention, can be a prokaryotic or eukaryotic cell, including, for example, a bacterial cell such as an E. coli cell; a plant cell such as an algae or a monocot or dicot; an insect cell; or a vertebrate cell such as a mammalian cell. Where the cell is a plant cell, the polynucleotide, or vector, can be contained in a plastid of the plant cell, particularly in a chloroplast, and can, but need not, be integrated into the plastid genome.

Generally, the polynucleotide of the invention, which can be contained in a vector, is operatively linked to an expressible polynucleotide, whereby the cell containing the polynucleotide provides an expression system, which allows the translation of one or more polypeptides encoded by the expressible polynucleotide. As such, the expressible polynucleotide, which can be biased for codon usage by the plastid, particularly chloroplast codon usage, encodes at least a first polypeptide, for example, a first polypeptide and a second polypeptide. In one embodiment, the expressible polynucleotide encodes an antibody. In another embodiment, the expressible polynucleotide is biased for chloroplast codon usage, for example, an expressible polynucleotide having a nucleotide sequence as set forth in SEQ ID NO:1, SEQ ID NO:13, SEQ ID NO:15, SEQ ID NO:42, SEQ ID NO:45, or SEQ ID NO:47.

The present invention further relates to a transgenic plant, which comprises plant cells containing a polynucleotide of the invention integrated in chloroplast genomic DNA. Accordingly, the present invention provides a plant cell organelle or a cell or tissue obtained from such a transgenic plant, for example, a chloroplast isolated from the transgenic plant, or leaves or flowers isolated from the transgenic plant, a fruit or rhizome isolated from the transgenic plant, or a cutting of the transgenic plant, or a seed produced by the transgenic plant. In addition, the invention provides cDNA or chloroplast genomic DNA library prepared from the transgenic plant of the invention, or from a plant cell or plant tissue obtained from the transgenic plant. A transgenic plant of the invention can be any type of plant, including, for example, an algae, which can be microalgae or a macroalgae; a monocot; or a dicot such as an angiosperm (e.g., a cereal plant, a leguminous plant, an oilseed plant, or a hardwood tree), including an ornamental plant.

The present invention further relates to a composition, which includes plant material obtained from a transgenic plant of the invention or from a plant cell genetically modified to contain a polynucleotide of the invention integrated in chloroplast genomic DNA of the plant. Preferably, the polynucleotide encoding the operatively linked first RBS and second RBS in the transgenic plant or genetically modified plant cell is operatively linked to an expressible polynucleotide, which can, but need not, be biased for chloroplast codon usage. As such, the plant material, which can be cell organelles, cells, or one or more tissues obtained from a transgenic plant, for example, chloroplasts, or leaves or flowers, a fruit or rhizome, or a seed produced by a transgenic plant, provides a source of the polypeptide or polypeptides encoded by the expressible polynucleotide. For example, where the expressible polynucleotide encodes an antibody, or an antigen binding fragment thereof, the plant material and, therefore, the composition, provides a source of the antibody.

A composition of the invention can be formulated such that it is in a form suitable for administration to a living subject, for example, a vertebrate or other mammal, which can be a domesticated animal or a pet, or can be a human. Accordingly, depending on the polypeptide or polypeptides encoded by the expressible polynucleotide, a composition comprising a plant material as disclosed herein can be useful as a nutritional supplement, a therapeutic agent, and the like. For example, where the expressible polynucleotide encodes an antibody, or antigen binding fragment thereof, the composition can be useful for passive immunization of a subject such as an individual exposed to a herpesvirus, or an individual exposed to tetanus toxin. As such, the present invention provides a medicament useful for ameliorating a pathologic condition such as a herpesvirus infection.

The present invention also relates to an isolated polynucleotide encoding a fluorescent protein or a mutant or variant thereof, wherein codons of the polynucleotide are biased to reflect chloroplast codon usage. The polynucleotide can be a DNA sequence or an RNA sequence, and can be single stranded or double stranded, and can be a linear polynucleotide containing a cloning site at one or both ends. The polynucleotide also can be operatively linked to a polynucleotide encoding a first RBS and a second RBS that are spaced apart by about 5 to 25 nucleotides, such that the fluorescent protein conveniently can be translated in a prokaryote and in a chloroplast.

One or more codons encoding a fluorescent protein of the invention can be biased, for example, to contain an adenine or a thymine at position three, thus facilitating translation of the fluorescent protein in a chloroplast. For example, the fluorescent protein can be a green fluorescent protein (GFP) such as that produced by a species of Aequorea jellyfish. Such polynucleotides of the invention are exemplified by polynucleotides that encodes the polypeptide set forth in SEQ ID NO:2, for example, the polynucleotide set forth in SEQ ID NO:1. Accordingly, the present invention also provides a fluorescent protein encoded by and expressed from such a polynucleotide, for example, a fluorescent protein having an amino acid sequence as set forth in SEQ ID NO:2.

The present invention further relates to a recombinant nucleic acid molecule, which includes a first polynucleotide, which encodes at least one polypeptide and contains one or more codons biased to reflect chloroplast codon usage; and a second polynucleotide, which comprises a nucleotide sequence encoding a first RBS operatively linked to a nucleotide sequence encoding a second RBS, wherein the first RBS can direct translation of the polypeptide in a prokaryote and the second RBS can direct translation of the polypeptide in a chloroplast. The first polynucleotide can encode a single polypeptide, or can encode two or more polypeptides, which can be expressed as separate polypeptides or as a fusion protein. Where the first polynucleotide encodes two or more polypeptides, the nucleotide sequence between the coding sequences can, but need not, encode an internal ribosome entry site, which is positioned so as to facilitate translation of the second (or other) polypeptide. A recombinant nucleic acid molecule of the invention can further include a third polynucleotide, which can be operatively linked to the first and second polynucleotides and can, but need not, encode one or more polypeptides.

The present invention also relates to a method of making a chloroplast/prokaryote shuttle expression vector. Such a method can be performed, for example, by introducing into a nucleotide sequence of chloroplast genomic DNA sufficient to undergo homologous recombination with chloroplast genomic DNA, a nucleotide sequence comprising a prokaryote origin of replication; a nucleotide sequence encoding a first RBS; and a nucleotide sequence encoding a second RBS, wherein the first RBS and second RBS are spaced apart by about 5 to 25 nucleotides; and a cloning site, wherein the cloning site is positioned to allow operative linkage of a polynucleotide encoding a polypeptide to the first RBS and second RBS such that the first RBS can direct translation of the polypeptide in a prokaryote and the second RBS can direct translation of the polypeptide in a chloroplast. A method of making a chloroplast/prokaryote shuttle expression vector also can be performed by genetically modifying a nucleotide sequence of chloroplast genomic deoxyribonucleic acid (DNA), which is sufficient to undergo homologous recombination with chloroplast genomic DNA, to contain a prokaryote origin of replication, a nucleotide sequence encoding a first RBS spaced apart from a second RBS by about 5 to 25 nucleotides, and a cloning site positioned to allow operative linkage of a polynucleotide encoding a polypeptide to the first RBS and second RBS such that the first RBS can direct translation of the polypeptide in a prokaryote and the second RBS can direct translation of the polypeptide in a chloroplast. Accordingly, the present invention also provides a chloroplast/prokaryote shuttle vector produced by a method as disclosed herein.

The present invention further relates to a recombinant polynucleotide, which includes a first nucleotide sequence encoding a chloroplast RBS operatively linked to at least a second nucleotide sequence encoding a polypeptide, wherein the first nucleotide sequence is heterologous with respect to the second nucleotide sequence. Such a recombinant polynucleotide can further include an operatively linked third (or more) nucleotide sequence encoding a second (or other) polypeptide, thus providing a recombinant polynucleotide encoding a dicistronic (or polycistronic) polyribonucleotide sequence. A nucleotide encoding an operatively linked RBS generally is positioned about 20 to 40 nucleotides 5′ (upstream) to an initiator ATG codon, which, in turn, is operatively linked to the nucleotide sequence encoding the polypeptide. In one embodiment, the first nucleotide sequence comprises an ATG codon positioned about 20 to 40 nucleotides 3′ of sequence encoding the RBS. In another embodiment, an internal ribosome binding sequence is operatively linked between two or more nucleotide sequences encoding polypeptides, which can be the same or different.

The present invention also relates to a vector, which includes a nucleotide sequence encoding an RBS positioned about 20 to 40 nucleotides 5′ to a cloning site. The cloning site can be any nucleotide sequence that facilitates insertion or linkage of a nucleotide sequence to the vector, for example, one or more restriction endonuclease recognition sites, one or more recombinase recognition sites, or a combination of such sites. Preferably, the cloning site is a multiple cloning site, which includes a plurality of restriction endonuclease recognition sites or recombinase recognition sites, or a combination of at least one restriction endonuclease recognition site and at least one recombinase recognition site. The vector can further contain an initiator ATG codon or a portion thereof adjacent and 5′ to the cloning site, thus providing a translation start site for a coding sequence that otherwise lacks an initiator ATG codon. In addition, the vector can contain a chloroplast gene 3′ untranslated region positioned 3′ to the cloning site.

### BRIEF DESCRIPTION OF THE FIGURES

FIG. 1 provides a comparison of the GFPct (SEQ ID NO: 1) and GFPncb (SEQ ID NO:3) coding regions. The amino acid sequence of GFPct (SEQ ID NO:2) is shown below the nucleotide sequence. Changed codons are boxed, and those that show a significant improvement in usage are shaded. The optimized codons were defined as codons used more than 10 times per 1000 codons in the C. reinhardtii chloroplast genome (Nakamura et al., Nucl. Acids Res. 27:292, 1999). The asterisk (*) indicates the two amino acid changes between GFPct and GFPncb, at positions 2 and 65.

FIG. 2 provides a characterization of pET expressed GFPct and GFPncb. GFPct and GFPncb proteins expressed from pET19b plasmid in E. coli were purified via Ni agarose affinity chromatography (Example 1). Crude E. coli lysates containing either GFPct or GFPncb proteins (20 μl) were prepared by subjecting samples to 12% SDS-PAGE without boiling, and disassembling the gel apparatus, but leaving the gel encased within the glass plates. Fluorescent gels were visualized with the indicated excitation (ex) and emission (em) filters. Five μg of affinity purified GFPct or GFPncb proteins were separated on a 12% SDS-PAGE and stained with Coomassie. 100 ng of affinity purified GFPct or GFPncb protein was subjected to 12% SDS PAGE followed by western blotting and detection with anti-GFP primary antibody. Excitation spectra were generated for affinity purified GFPct (4 μg), and GFPncb (20 μg) proteins. Relative fluorescence was recorded at excitation from 350 to 550 nm with emission fixed at 510 nm. The GFPncb (stippled line) and GFPct (solid line) excitation spectra are shown; dashed line represents the 510 nm emission peak seen in both samples.

FIG. 3 provides maps of the GFPct and GFPncb reporter gene used for expression in C. reinhardtii chloroplasts.

FIG. 3A shows relevant restriction sites delimiting the rbcL 5′ UTR (Bam HI/Nde I; see, also, SEQ ID NO:5) from either GFPct (SEQ ID NO: 1) or GFPncb (SEQ ID NO:3) coding regions (NdeI/Xba I) and the rbcL 3′UTR (Xba I/Bam HI; see, also, SEQ ID NO: 10). Size of each fragment in base pairs (bp) is indicated.

FIG. 3B shows the site of integration into the C. reinhardtii chloroplast genome of the GFPct and GFPncb genes under control of the rbcL 5′ and 3′ UTRs. C. reinhardtii chloroplast DNA is depicted as the Eco RI to Xho I fragment of 5.7 kb. Double headed arrows indicate regions corresponding to the probes used in the Southern blot analysis.

FIG. 4 shows the linear sequence of the mutant psbA 5‘UTR’s (SEQ ID NOS:35 to 41) corresponding to positions +3 to −36 relative to the initiation codon of the wild type 5′UTR (SEQ ID NO:34). The 5′UTR's were placed upstream of the DI cDNA, which is an intron-less copy of the wild type psbA gene. Changes to the primary sequence are underlined and the initiation codons are boxed. The * denotes the 5′ terminus of the mRNA in vivo resulting from a processing event that cleaves the 5′UTR (see Bruick and Mayfield, Trends Plant Sci. 4:190-195, 1998, which is incorporated herein by reference).

FIGS. 5A to 5C provide restriction maps of HSV8-lsc genes for expression in C. reinhardtii chloroplasts. HSV8-lsc nucleotide (SEQ ID NO:47) and amino acid (SEQ ID NO:48) sequences are provided in the Sequence Listing.

FIGS. 5A and 5B show relevant restriction sites delineating the rbcL 5′UTR (Bam HI/Nde I), the HSV8 coding region and flag tag (NdeI/Xba I), and the rbcL 3′UTR (Xba I/Bam HI; FIG. 5A), as well as relevant restriction sites of the atpA 5′UTR (Bam HI/Nde I), the HSV8 coding region and flag tag (NdelI/Xba I), and the rbcL 3′UTR (Xba I/Bam HI; FIG. 5B).

FIG. 5C provides a restriction map showing the site of integration of the HSV8-lsc genes into plasmid p322 for integration into the C. reinhardtii chloroplast genome. p322 DNA includes the 5.7 kb region from Eco RI to Xho I in the C. reinhardtii chloroplast genome corresponding to position 44,877 to 50,577 (see world wide web at URL “biology.duke.edu/chlamy_genome/chloro.html”). Double headed arrows indicate regions corresponding to the probes used in the Southern blot analysis. Black boxes indicate (from left to right) psbA exon 5, and the 5S and a small portion of the 23S ribosomal RNA genes, respectively.

FIG. 6 provides a characterization of HSV8-lsc binding to HSV8 viral protein obtained by ELISA. Affinity purified HSV8-lsc from the transgenic C. reinhardtii strains (10-6-3 and 16-3) were screened in an ELISA assay against HSV proteins prepared from virus infected cells. 100, 80, 70, 60, 30, 20, 10 or 5 μl of Flag purified HSV8-lsc were incubated in microtiter plates coated with a constant amount of viral protein. Protein concentrations in these affinity purified extracts was 13 ng/μl, of which approximately 10% was HSV8-lsc as judged by Coomassie staining. Equal volumes of wt C. reinhardtii proteins were used as a negative control (concentration 1 μg/μl).

FIG. 7 provides a comparison of the luxAB (SEQ ID NO:44) and luxCt (amino acid residues 2 to 695 of SEQ ID NO:46) coding regions. The amino acid sequence is shown with the modified codons indicated by boxed and shaded amino acids. The optimized codons were defined as codons used more than 10 times per 1000 codons in the C. reinhardtii chloroplast genome (Nakamura et al. 1999). The amino acid differences between the two proteins are indicated by boxed and unshaded amino acids, and the two amino acids changed that resulted in active luciferase are indicated by the ** above the changed amino acids.

FIGS. 8A and 8B provide maps of luxCt gene for expression in C. reinhardtii chloroplasts.

FIG. 8A illustrates relevant restriction sites delineating the atpA 5′ UTR (Bam HI/Nde I), the luxCt coding region (NdeI/Xba I) and the rbcL 3′ UTR (Xba I/Bam HI).

FIG. 8B provides a map showing the homologous region between plasmid p322 and the C. reinhardtii chloroplast genome into which the chimeric luxCt gene was inserted. C. reinhardtii chloroplast DNA depicted is the Eco RI to Xho I fragment of 5.7 kb located in the inverted repeat region of the chloroplast region. Double headed arrows indicate regions corresponding to the probes used in the Southern and Northern blot analysis. Black boxes indicate, from 1 to r, psbA exon 5, 5s rRNA and 23s RNA genes, respectively.

### DETAILED DESCRIPTION OF THE INVENTION

The present provides compositions and methods for expressing functional polypeptides, including functional protein complexes, in plastids, particularly in plant chloroplasts, as well as compositions that facilitate transfer of polynucleotides among plant chloroplasts and prokaryotes and allow expression of encoded polypeptides in the chloroplasts and prokaryotes. In one embodiment, a method of the invention is exemplified by expressing functional antibodies, including single chain antibodies that properly assemble and function to specifically bind antigen, as well as antibodies and antigen binding fragments thereof that are expressed as single chains and that specifically associate to form homodimers that specifically bind antigen.

According to one method of the invention, the polynucleotides encoding the antibodies are operatively linked to a 5′-untranslated region (5′UTR) comprising a ribosome binding sequence (RBS) that directs translation of the antibodies in chloroplasts. In another embodiment, the polynucleotides encoding the antibodies are operatively linked to a first RBS, which directs translation in a prokaryotic cell, and a second RBS, which directs translation in a chloroplast. In still another embodiment, the polynucleotide encoding an antibody is biased for chloroplast codon usage.

According to another method of the invention, a synthetic polynucleotide, which includes at least a first nucleotide sequence encoding at least a first polypeptide, wherein at least one codon in the first nucleotide sequence is biased to reflect chloroplast codon usage, is introduced into a cell, wherein the encoded polypeptide is expressed. In one embodiment, each codon in the first nucleotide sequence is biased to reflect chloroplast codon usage, and in another embodiment, the synthetic polynucleotide contains at least a second nucleotide sequence, which can, but need not, be operatively linked to the first nucleotide sequence, and encodes at least a second polypeptide, wherein expression of the polynucleotide can, but need not, generate a fusion protein comprising the first and second (or more) polypeptides. Accordingly, a synthetic polynucleotide, which includes at least a first nucleotide sequence encoding at least a first polypeptide, wherein at least one codon in the first nucleotide sequence is biased to reflect chloroplast codon usage, is provided. As used herein, the term “synthetic polynucleotide” means a nucleic acid molecule that has been modified by changing a codon in the polypeptide that is not biased for chloroplast codon usage to a codon that is biased for chloroplast codon usage (see Table 1, below). As disclosed herein, polypeptides encoded by such synthetic polynucleotides are robustly expressed in chloroplasts.

In other embodiments, compositions for practicing a method of the invention are provided. Advantages provided by the present invention include the ability to obtain robust expression of functional polypeptides in plant chloroplasts, wherein the polypeptides are not glycosylated and, therefore, have reduced antigenicity upon administration to a subject, as well as the ability to produce large amounts of functional polypeptides without a requirement for a fermentation facility and the expense associated therewith.

A method of the invention provides a means to express one or more polypeptides in a plant chloroplast, whereby the polypeptides can assemble to produce functional protein complexes. As disclosed herein, polypeptides expressed in chloroplasts not only assemble properly, but also, where the polypeptides comprise subunits of a protein complex, the polypeptides can specifically associate to produce a functional protein complex. As used herein, the term “protein complex” refers to a composition that is formed by the specific association of at least two polypeptides, which can be the same or different. Polypeptides that specifically associate to function as protein complexes are well known and include enzymes, growth factors, growth factor and hormone receptors, and the like.

As used herein, the term “specifically associate” or “specifically interact” or “specifically bind” refers to two or more polypeptides that form a complex that is relatively stable under physiologic conditions. The terms are used herein in reference to various interactions, including, for example, the interaction of a first polypeptide subunit and a second polypeptide subunit that interact to form a functional protein complex, as well as to the interaction of an antibody and its antigen. A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a cell or subcellular compartment of a living subject, including a plant or an animal, which can be a vertebrate or invertebrate, as well as conditions that occur in a cell culture such as used for maintaining cells or tissues of an organism. Methods for determining whether two molecules interact specifically are well known and include, for example, equilibrium dialysis, surface plasmon resonance, gel shift analyses, and the like.

The usefulness of a method of the invention to produce functional polypeptides, including functional protein complexes, is exemplified herein by the production of functional antibodies. The term “antibody” is used broadly herein to refer to a polypeptide or a protein complex that can specifically bind an epitope of an antigen. Generally, an antibody contains at least one antigen binding domain that is formed by an association of a heavy chain variable region domain and a light chain variable region domain, particularly the hypervariable regions. An antibody generated according to a method of the invention can be based on naturally occurring antibodies, for example, bivalent antibodies, which contain two antigen binding domains formed by first heavy and light chain variable regions and second heavy and light chain variable regions (e.g., an IgG or IgA isotype) or by a first heavy chain variable region and a second heavy chain variable region (V_{HH} antibodies; see, for example, U.S. Pat. No. 6,005,079), or on non-naturally occurring antibodies, including, for example, single chain antibodies, chimeric antibodies, bifunctional antibodies, and humanized antibodies, as well as antigen-binding fragments of an antibody, for example, an Fab fragment, an Fd fragment, an Fv fragment, and the like.

In one embodiment, a method of the invention is exemplified using a polynucleotide encoding a single chain antibody comprising a heavy chain operatively linked to a light chain, wherein the antibody specifically binds tetanus toxin (see SEQ ID NOS:13 and 14). In another embodiment, the method is exemplified using a polynucleotide encoding a single chain antibody comprising a heavy chain operatively linked to a light chain, wherein the antibody specifically binds herpes simplex virus types 1 and 2, and wherein the polynucleotide encoding the antibody is biased for chloroplast codon usage (see SEQ ID NOS:15 and 16; SEQ ID NOS:42 and 43; and SEQ ID NOS:47 and 48; see, also, Example 3).

Polynucleotides useful for practicing a method of the invention can be isolated from cells producing the antibodies of interest, for example, B cells from an immunized subject or from an individual exposed to a particular antigen, can be synthesized de novo using well known methods of polynucleotide synthesis, can be produced recombinantly or can be obtained, for example, by screening combinatorial libraries of polynucleotides that encode variable heavy chains and variable light chains (see Huse et al., Science 246:1275-1281 (1989), which is incorporated herein by reference), and can be biased for chloroplast codon usage, if desired (see Example 1, and Table 1). These and other methods of making polynucleotides encoding, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference).

Polynucleotides encoding humanized monoclonal antibodies, for example, can be obtained by transferring nucleotide sequences encoding mouse complementarity determining regions from heavy and light variable chains of the mouse immunoglobulin gene into a human variable domain gene, and then substituting human residues in the framework regions of the murine counterparts. General techniques for cloning murine immunoglobulin variable domains are known (see, for example, Orlandi et al., Proc. Natl. Acad. Sci., USA 86:3833, 1989, which is hereby incorporated in its entirety by reference), as are methods for producing humanized monoclonal antibodies (see, for example, Jones et al., Nature 321:522, 1986; Riechmann et al., Nature 332:323, 1988; Verhoeyen et al., Science 239:1534, 1988; Carter et al., Proc. Natl. Acad. Sci., USA 89:4285, 1992; Sandhu, Crit. Rev. Biotechnol. 12:437, 1992; and Singer et al., J. Immunol. 150:2844, 1993; each of which is incorporated herein by reference).

The methods of the invention also can be practiced using polynucleotides encoding human antibody fragments isolated from a combinatorial immunoglobulin library (see, for example, Barbas et al., Methods: A Companion to Methods in Immunology 2:119, 1991; Winter et al., Ann. Rev. Immunol. 12:433, 1994; each of which is incorporated herein by reference). Cloning and expression vectors that are useful for producing a human immunoglobulin phage library can be obtained, for example, from Stratagene Cloning Systems (La Jolla, Calif.).

A polynucleotide encoding a human monoclonal antibody also can be obtained, for example, from transgenic mice that have been engineered to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas, from which polynucleotides useful for practicing a method of the invention can be obtained. Methods for obtaining human antibodies from transgenic mice are described, for example, by Green et al., Nature Genet. 7:13, 1994; Lonberg et al., Nature 368:856, 1994; and Taylor et al., Intl. Immunol. 6:579, 1994; each of which is incorporated herein by reference, and such transgenic mice are commercially available (Abgenix, Inc.; Fremont Calif.).

The polynucleotide also can be one encoding an antigen binding fragment of an antibody. Antigen binding antibody fragments, which include, for example, Fv, Fab, Fab′, Fd, and F(ab′)₂ fragments, are well known in the art, and were originally identified by proteolytic hydrolysis of antibodies. For example, antibody fragments can be obtained by pepsin or papain digestion of whole antibodies by conventional methods. Antibody fragments produced by enzymatic cleavage of antibodies with pepsin generate a 5S fragment denoted F(ab′)₂. This fragment can be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647, each of which is incorporated by reference, and references contained therein; Nisonhoff et al., Arch. Biochem. Biophys. 89:230. 1960; Porter, Biochem. J. 73:119, 1959; Edelman et al., Meth. Enzymol. 1:422 (Academic Press 1967); Coligan et al., In Curr. Protocols Immunol., 1992, see sections 2.8.1-2.8.10 and 2.10.1-2.10.4; each of which is incorporated herein by reference).

Another form of an antibody fragment is a peptide coding for a single complementarity-determining region (CDR). CDR peptides can be obtained by constructing a polynucleotide encoding the CDR of an antibody of interest, for example, by using the polymerase chain reaction to synthesize the variable region from RNA of antibody-producing cells (see, for example, Larrick et al., Methods: A Companion to Methods in Enzymology 2:106, 1991, which is incorporated herein by reference). Polynucleotides encoding such antibody fragments, including subunits of such fragments and peptide linkers joining, for example, a heavy chain variable region and light chain variable region, can be prepared by chemical synthesis methods or using routine recombinant DNA methods, beginning with polynucleotides encoding full length heavy chains and light chains, which can be obtained as described above.

The present methods are based, in part, on the determination that proper positioning of a ribosome binding sequence (RBS) with respect to a coding sequence results in robust translation in plant chloroplasts (see below; see, also, Example 2), and that polypeptides that are known to specifically associate to form protein complexes when produced naturally in an organism (e.g., antibodies) also can associate properly in chloroplasts (see Example 3). An advantage of expressing such polypeptides in chloroplasts is that the polypeptides do not proceed through cellular compartments typically traversed by polypeptides expressed from a nuclear gene and, therefore, are not subject to certain post-translational modifications such as glycosylation. As such, the polypeptides and protein complexes produced by a method of the invention can be expected to be less antigenic than the same polypeptides would be if expressed from a polynucleotide introduced into the nucleus of a eukaryote.

A method of the invention provides a means to produce functional polypeptides such as single chain antibodies, and protein complexes such as a bivalent antibody, which include, for example, a first heavy and light chain associated with a second heavy and light chain. As disclosed herein, a method of the invention can be performed, for example, by introducing a recombinant nucleic acid molecule into a chloroplast, wherein the recombinant nucleic acid molecule includes a first polynucleotide, which encodes at least one polypeptide (i.e., 1, 2, 3, 4, or more), operatively linked to a second polynucleotide, which includes a nucleotide sequence encoding a first RBS operatively linked to a nucleotide sequence encoding a second RBS, under conditions that allow expression of the at least one polypeptide. Such conditions include those that allow or facilitate entry of the recombinant nucleic acid molecule into the chloroplast and, preferably, incorporation of the recombinant nucleic acid molecule into the chloroplast genome. Such methods include those exemplified herein, as well as other methods known and routine in the art.

As used herein, the term “operatively linked” means that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated with in a cell. A first polynucleotide coding sequence also can be operatively linked to a second (or more) coding sequence such that a chimeric polypeptide can be expressed from the operatively linked coding sequences (see, for example, SEQ ID NO:30, showing site where polynucleotide, which encodes a GFP and was biased for chloroplast codon usage (i.e., SEQ ID NO: 1) was inserted into the PsbD gene, such that a fluorescent fusion protein comprising the PsbD gene product fused to GFP was generated). The chimeric polypeptide can be a fusion polypeptide, in which the two (or more) encoded peptides are translated into a single polypeptide, i.e., are covalently bound through a peptide bond, for example, a single chain antibody comprising a heavy chain variable region operatively linked (through a linker peptide, if desired) to a light chain variable region; or can be translated as two discrete peptides that, upon translation, can specifically associate with each other to form a stable protein complex, for example, an antibody heavy chain and an antibody light chain, which form a quaternary structure resulting in a functional monovalent antibody, and which can further associate to produce a functional bivalent antibody. Examples of synthetic polynucleotides encoding such fusion proteins include SEQ ID NO:45, which encodes a bacterial luciferase fusion protein, and SEQ ID NOS: 15, 42, and 47, which encode single chain anti-HSV antibodies.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition, except that the term “synthetic polynucleotide” as used herein refers to a polynucleotide that has been modified to reflect chloroplast codon usage.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available (e.g., Ambion, Inc.; Austin Tex.), as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference). The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995, each of which is incorporated herein by reference).

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

The term “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element, particularly a first RBS operatively linked to a second RBS. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

As disclosed herein, positioning of an RBS about 20 to 40 nucleotides upstream (5′) of an initiation codon, for example, an AUG codon, allows robust translation of coding sequence starting with the AUG codon (see Example 2). As such, an RBS positioned about 20 to 40 nucleotides upstream of an AUG codon is considered “operatively linked” to the AUG codon. Furthermore, it is well known that an RBS positioned about 5 to 15 nucleotides upstream from an initiation codon can direct translation of a coding sequence in prokaryotes and, as disclosed herein, such an RBS can be operatively linked to a second RBS positioned about 20 to 40 nucleotides upstream of the initiation codon to produce a translational regulatory element than can direct translation in a prokaryote and in a chloroplast. As such, a first and second RBS spaced apart by about 5 to 25 nucleotides are considered operatively linked with respect to each other. It should be recognized that the terms “first”, “second”, “third”, etc., when used herein in reference to an RBS or a polynucleotide or polypeptide or the like, are used only for convenience of discussion and, unless specifically indicated otherwise, do not imply an order, importance, or the like. As such, while reference is made herein, for example, to a first RBS that can direct translation in a prokaryote and a second RBS that can direct translation in a chloroplast, the designations “first” and “second” (and the like) are made only to conveniently distinguish the two (or more) elements.

Reference to an RBS having the ability to “direct translation” means that, when operatively linked to a coding sequence, which generally begins with an initiation codon, the RBS can be bound by a ribosome such that translation can occur beginning with the initiation codon. As used herein, the term “initiation codon” refers to a ribonucleotide sequence or encoding deoxyribonucleotide sequence that is the first codon of a coding sequence. Generally, an initiation codon is an “initiator AUG codon” (in RNA) or an “initiator ATG codon” (in DNA), and encodes methionine, although other codons also can act as an initiation codons, including, for example, CUG.

One or more codons of an encoding polynucleotide can be biased to reflect chloroplast codon usage (Example 1). Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. Table 1 (below) shows the chloroplast codon usage for C. reinhardtii.

The term “biased”, when used in reference to a codon, means that the sequence of a codon in a polynucleotide has been changed such that the codon is one that is used preferentially in chloroplasts (see Table 1). A polynucleotide that is biased for chloroplast codon usage can be synthesized de novo, or can be genetically modified using routine recombinant DNA techniques, for example, by a site directed mutagenesis method, to change one or more codons such that they are biased for chloroplast codon usage (see Example 1). As disclosed herein, chloroplast codon bias can be variously skewed in different plants, including, for example, in alga chloroplasts as compared to tobacco. Generally, the chloroplast codon bias selected for purposes of the present invention, including, for example, in preparing a synthetic polynucleotide as disclosed herein,

TABLE 1
Chloroplast Codon Usage in Chlamydomonas reinhardtii -

| | | | |
|---|---|---|---|
| UUU 34.1*( 348**) | UCU 19.4( 198) | UAU 23.7( 242) | UGU 8.5( 87) |
| UUC 14.2( 145) | UCC 4.9( 50) | UAC 10.4( 106) | UGC 2.6( 27) |
| UUA 72.8( 742) | UCA 20.4( 208) | UAA 2.7( 28) | UGA 0.1( 1) |
| UUG 5.6( 57) | UCG 5.2( 53) | UAG 0.7( 7) | UGG 13.7( 140) |
| CUU 14.8( 151) | CCU 14.9( 152) | CAU 11.1( 113) | CGU 25.5( 260) |
| CUC 1.0( 10) | CCC 5.4( 55) | CAC 8.4( 86) | CGC 5.1( 52) |
| CUA 6.8( 69) | CCA 19.3( 197) | CAA 34.8( 355) | CGA 3.8( 39) |
| CUG 7.2( 73) | CCG 3.0( 31) | CAG 5.4( 55) | CGG 0.5( 5) |
| AUU 44.6( 455) | ACU 23.3( 237) | AAU 44.0( 449) | AGU 16.9( 172) |
| AUC 9.7( 99) | ACC 7.8( 80) | AAC 19.7( 201) | AGC 6.7( 68) |
| AUA 8.2( 84) | ACA 29.3( 299) | AAA 61.5( 627) | AGA 5.0( 51) |
| AUG 23.3( 238) | ACG 4.2( 43) | AAG 11.0( 112) | AGG 1.5( 15) |
| GUU 27.5( 280) | GCU 30.6( 312) | GAU 23.8( 243) | GGU 40.0( 408) |
| GUC 4.6( 47) | GCC 11.1( 113) | GAC 11.6( 118) | GGC 8.7( 89) |
| GUA 26.4( 269) | GCA 19.9( 203) | GAA 40.3( 411) | GGA 9.6( 98) |
| GUG 7.1( 72) | GCG 4.3( 44) | GAG 6.9( 70) | GGG 4.3( 44) |

* - Frequency of codon usage per 1,000 codons.
** - Number of times observed in 36 chioroplast coding sequences (10,193 codons).

reflects chloroplast codon usage of a plant chloroplast, and includes a codon bias that, with respect to the third position of a codon, is skewed towards A/T, fore example, where the third position has greater than about 66% AT bias, particularly greater than about 70% AT bias. As such, chloroplast codon biased for purposes of the present invention excludes the third position bias observed, for example, in Nicotiana tabacus (tobacco), which has 34.56% GC bias in the third codon position (see, for example, world wide web at URL “kazusa.or.jp/codon/”, and the “chloroplast” link). In one embodiment, the chloroplast codon usage is biased to reflect alga chloroplast codon usage, for example, C. reinhardtii, which has about 74.6% AT bias in the third codon position.

A method of the invention can be performed using a polynucleotide that encodes a first polypeptide and at least a second polypeptide. As such, the polynucleotide can encode, for example, a first polypeptide and a second polypeptide; a first polypeptide, a second polypeptide, and a third polypeptide; etc. Furthermore, any or all of the encoded polypeptides can be the same or different. As disclosed herein, polypeptides expressed in chloroplasts of the microalga Chlamydomonas reinhardtii assembled to form functional polypeptides and protein complexes (see Examples 1 and 3). As such, a method of the invention provides a means to produce functional protein complexes, including, for example, dimers, trimers, and tetramers, wherein the subunits of the complexes can be the same or different (e.g., homodimers or heterodimers, respectively).A method of expressing functional polypeptides and protein complexes in chloroplasts is exemplified by the production of antibodies, and by the production of reporter proteins, including a green fluorescent protein and a luciferase (luxAB fusion protein; see Examples 1 and 4; see, also, SEQ ID NOS:1 and 45, respectively), and of an antibodies expressed from polynucleotides biased for chloroplast codon usage (see Example 3; see, also, SEQ ID NOS:15, 42, and 47). As exemplified herein, chloroplasts were transfected with a recombinant nucleic acid molecule comprising a polynucleotide encoding a single chain antibody having a complete heavy chain linked to a light chain variable region, wherein homodimers comprising two single chain antibodies that associated through a specific interaction of their heavy chain domains were produced. These results provide the first evidence that heterologous polypeptides can specifically associate to form quaternary structures in chloroplasts, and demonstrate that heteropolymers can be produced, according to a method of the invention, by introducing into chloroplasts a single recombinant nucleic acid molecule encoding each of the different polypeptides of the heteropolymer, or by introducing two or more polynucleotides, each of which encodes one (or more) subunits of the heteropolymer.

A method of the invention can be practiced using a first recombinant nucleic acid molecule, which includes a nucleotide sequence encoding an RBS that directs translation in chloroplasts, and, preferably, further encoding an operatively linked RBS that directs translation in a prokaryote, the nucleotide sequence being operatively linked to at least one polynucleotide encoding at least a first polypeptide. For example, the recombinant nucleic acid molecule can include a polynucleotide encoding an immunoglobulin heavy chain (H) or a variable region thereof (V_{H}), and can further encode a second polypeptide, which is an immunoglobulin light chain (L) or a variable region thereof (V_{L}). If desired, a nucleotide sequence encoding an internal ribosome entry site can be positioned between the nucleotide sequences encoding the H and L chains such that expression of the second (downstream) encoded polypeptide is facilitated. Upon translation of the encoded H and L chains in the chloroplast, a H chain can associate with a L chain to form a monovalent antibody (i.e., an H:L complex), and two H:L complexes can further associate to produce a bivalent antibody.

A method of the invention also can be practiced by introducing into a plant chloroplast a first recombinant nucleic acid molecule, which includes a polynucleotide encoding, for example, a H chain or a V_{H} chain, and further introducing into the chloroplast as second recombinant nucleic acid molecule, which includes a polynucleotide encoding a L chain or a V_{L} chain, wherein each recombinant nucleic acid molecule includes a nucleotide sequence encoding a first RBS operatively linked to a nucleotide sequence encoding a second RBS, wherein the first RBS can direct translation of the polypeptide in a prokaryote and the second RBS can direct translation of the polypeptide in a chloroplast, and wherein the nucleotide sequence encoding the two RBS is operatively linked to the encoding polynucleotide sequence. Where the plant cells containing the chloroplasts are exposed to conditions that allow encoded polypeptides to be co-expressed the H chains and L chains can associate to form an H:L complex, and the H:L complexes can further associate to produce a bivalent antibody.

A recombinant nucleic acid molecule comprising a polynucleotide encoding a polypeptide can further contain, operatively linked to the coding sequence, a peptide tag such as a His-6 tag or the like, which can facilitate identification of expression of the polypeptide in a cell. A polyhistidine tag peptide such as His-6 can be detected using a divalent cation such as nickel ion, cobalt ion, or the like. Additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S-transferase, which can be detected using glutathione. Such tags can provide the additional advantage that they can facilitate isolation of the operatively linked polypeptide, for example, where it is desired to obtain a substantially purified polypeptide.

A recombinant nucleic acid molecule useful in a method of the invention can be contained in a vector. Furthermore, where the method is performed using a second (or more) recombinant nucleic acid molecules, the second recombinant nucleic acid molecule also can be contained in a vector, which can, but need not, be the same vector as that containing the first recombinant nucleic acid molecule. The vector can be any vector useful for introducing a polynucleotide into a chloroplast and, preferably, includes a nucleotide sequence of chloroplast genomic DNA that is sufficient to undergo homologous recombination with chloroplast genomic DNA, for example, a nucleotide sequence comprising about 400 to 1500 or more substantially contiguous nucleotides of chloroplast genomic DNA. Chloroplast vectors and methods for selecting regions of a chloroplast genome for use as a vector are well known (see, for example, Bock, J. Mol. Biol. 312:425-438, 2001; see, also, Staub and Maliga, Plant Cell 4:39-45, 1992; Kavanagh et al., Genetics 152:1111-1122, 1999, each of which is incorporated herein by reference).

The entire chloroplast genome of C. reinhardtii is available to the public on the world wide web, at the URL “biology.duke.edu/chlamy_genome/chloro.html” (see “view complete genome as text file” link and “maps of the chloroplast genome” link), each of which is incorporated herein by reference (J. Maul, J. W. Lilly, and D. B. Stern, unpublished results; revised Jan. 28, 2002; to be published as GenBank Acc. No. AF396929). Generally, the nucleotide sequence of the chloroplast genomic DNA is selected such that it is not a portion of a gene, including a regulatory sequence or coding sequence, particularly a gene that, if disrupted due to the homologous recombination event, would produce a deleterious effect with respect to the chloroplast, for example, for replication of the chloroplast genome, or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector of the invention. For example, the chloroplast vector, p322, which was used in experiments disclosed herein, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlamy/chloro/chlorol40.html”; see, also, Example 1).

The vector also can contain any additional nucleotide sequences that facilitate use or manipulation of the vector, for example, one or more transcriptional regulatory elements, a sequence encoding a selectable markers, one or more cloning sites, and the like. In one embodiment, the chloroplast vector contains a prokaryote origin of replication (ori), for example, an E. coli ori, thus providing a shuttle vector that can be passaged and manipulated in a prokaryote host cell as well as in a chloroplast.

The methods of the present invention are exemplified using the microalga, C. reinhardtii. The use of microalgae to express a polypeptide or protein complex according to a method of the invention provides the advantage that large populations of the microalgae can be grown, including commercially (Cyanotech Corp.; Kailua-Kona HI), thus allowing for production and, if desired, isolation of large amounts of a desired product. However, the ability to express, for example, functional mammalian polypeptides, including protein complexes, in the chloroplasts of any plant allows for production of crops of such plants and, therefore, the ability to conveniently produce large amounts of the polypeptides. Accordingly, the methods of the invention can be practiced using any plant having chloroplasts, including, for example, macroalgae, for example, marine algae and seaweeds, as well as plants that grow in soil, for example, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals such as azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum are also included. Additional ornamentals useful for practicing a method of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Leguminous plants useful for practicing a method of the invention include beans and peas. Beans include guar, locust bean, fenugreek, soybean, garden beans, cowpea, mung bean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop. Other plants useful in the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, chicory, groundnut and zucchini.

A method of the invention can generate a plant containing chloroplasts that are genetically modified to contain a stably integrated polynucleotide (i.e., transplastomes; see, for example, Hager and Bock, Appl. Microbiol. Biotechnol. 54:302-310, 2000, which is incorporated herein by reference; see, also, Bock, supra, 2001). The integrated polynucleotide can comprise, for example, an encoding polynucleotide operatively linked to a first and second RBS as defined herein. Accordingly, the present invention further provides a transgenic (transplastomic) plant, which comprises one or more chloroplasts containing a polynucleotide encoding one or more heterologous polypeptides, including polypeptides that can specifically associate to form a functional protein complex. A transgenic plant comprising a transplastome provides advantages over transgenic plants having a polynucleotide integrated in the nuclear genome. For example, in most crop species, chloroplasts are strictly maternally inherited through the egg; the pollen (sperm) lacks chloroplasts (see, for example, Hager and Bock, supra, 2000). As such, a transgenic plant comprising a transplastome is unable to cross-pollinate other plants, including native plants that may be in the vicinity of the transgenic plant, thus reducing any potential ecological risk associated with the growth of transgenic plants in the environment.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

A transgenic plant can be regenerated from a transformed plant cell containing genetically modified chloroplasts. As used herein, the term “regenerate” means growing a whole plant from a plant cell; a group of plant cells; a protoplast; a seed; or a piece of a plant such as a callus or tissue. Regeneration from protoplasts varies from species to species of plants. For example, a suspension of protoplasts can be made and, in certain species, embryo formation can be induced from the protoplast suspension, to the stage of ripening and germination. The culture media generally contains various components necessary for growth and regeneration, including, for example, hormones such as auxins and cytokinins; and amino acids such as glutamic acid and proline, depending on the particular plant species. Efficient regeneration will depend, in part, on the medium, the genotype, and the history of the culture. If these variables are controlled, however, regeneration is reproducible.

Regeneration can occur from plant callus, explants, organs or plant parts. Transformation can be performed in the context of organ or plant part regeneration. (see Meth. Enzymol. Vol. 118; Klee et al. Ann. Rev. Plant Physiol. 38:467, 1987, which is incorporated herein by reference). Utilizing the leaf disk-transformation-regeneration method, for example, disks are cultured on selective media, followed by shoot formation in about two to four weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated utilizing cuttings or tissue culture techniques to produce multiple identical plants. Selection of desirable transgenotes is made and new varieties are obtained and propagated vegetatively for commercial use. In seed propagated crops, the mature transgenic plants can be self crossed to produce a homozygous inbred plant. The resulting inbred plant produces seeds that contain the introduced heterologous polynucleotide, and can be grown to produce plants that express a polypeptide encoded by the polynucleotide. As such, the invention further provides seeds produced by a transgenic plant obtained by a method of the invention.

If desired, transgenic plants of the invention containing chloroplasts that are genetically modified to express different heterologous polypeptides can be crossbred, thereby providing a means to obtain transgenic plants containing two or more different transgenes. Methods for breeding plants and selecting for crossbred plants having desirable characteristics or other characteristics of interest are well known in the art.

A method of producing a heterologous polypeptide or protein complex in a chloroplast or in a transgenic plant of the invention can further include a step of isolating an expressed polypeptide or protein complex from the plant cell chloroplasts. As used herein, the term “isolated” or “substantially purified” means that a polypeptide or polynucleotide being referred to is in a form that is relatively free of proteins, nucleic acids, lipids, carbohydrates or other materials with which it is naturally associated. Generally, an isolated polypeptide (or polynucleotide) constitutes at least twenty percent of a sample, and usually constitutes at least about fifty percent of a sample, particularly at least about eighty percent of a sample, and more particularly about ninety percent or ninety-five percent or more of a sample.

The term “heterologous” is used herein in a comparative sense to indicate that a nucleotide sequence (or polypeptide) being referred to is from a source other than a reference source, or is linked to a second nucleotide sequence (or polypeptide) with which it is not normally associated, or is modified such that it is in a form that is not normally associated with a reference material. For example, a polynucleotide encoding an antibody is heterologous with respect to a nucleotide sequence of a plant chloroplast, as are the components of a recombinant nucleic acid molecule comprising, for example, a first nucleotide sequence operatively linked to a second nucleotide sequence, as is a mutated polynucleotide introduced into a chloroplast where the mutant polynucleotide is not normally found in the chloroplast.

A polypeptide or protein complex can be isolated from chloroplasts using any method suitable for the particular polypeptide or protein complex, including, for example, salt fractionation methods and chromatography methods such as an affinity chromatography method using a ligand or receptor that specifically binds the polypeptide or protein complex. A determination that a polypeptide or protein complex produced according to a method of the invention is in an isolated form can be made using well known methods, for example, by performing electrophoresis and identifying the particular molecule as a relatively discrete band or the particular complex as one of a series of bands. Accordingly, the present invention also provides an isolated polypeptide or protein complex produced by a method of the invention.

The present invention also provides compositions that can be used alone or in combination to obtain robust expression of heterologous polypeptides in a chloroplast. In one embodiment, the invention provides a nucleotide sequence comprising (or encoding) a first RBS and a second RBS, wherein the first and second RBS are spaced apart such that one RBS directs translation in prokaryotic cells and the other RBS directs translation in plant chloroplasts. In one aspect, the nucleotide sequence also can contain (or encode) an initiation codon, for example, an initiator AUG (or ATG) codon, operatively linked to the first RBS and second RBS, or can contain a cloning site positioned so as to permit operative linkage of a coding sequence to the first and second RBS. In another aspect, the nucleotide sequence is contained in a vector, which, preferably, includes a nucleotide sequence of chloroplast genomic DNA that is sufficient to undergo site specific homologous recombination with a chloroplast genome. In still another aspect, the vector is a shuttle vector that further contains a prokaryote origin of replication.

In another embodiment, codon selection is utilized to bias an encoding polynucleotide for chloroplast codon usage, thus providing a means to obtain robust expression of one or more encoded polypeptides in a chloroplast. The usefulness of codon selection to optimize polypeptide expression in chloroplasts is exemplified herein using the Aequeoria victoria green fluorescent protein (GFP; Example 1). As such, the present invention also provides a polynucleotide encoding a GFP, wherein the polynucleotide has been codon optimized for expression in chloroplasts. As disclosed herein, the variant polynucleotide encodes a GFP that expressed in an amount making it useful as a reagent for detecting plant chloroplasts, including for examining gene expression in chloroplasts. The general usefulness of chloroplast codon optimization for expressing polypeptides is further demonstrated by the preparation of a synthetic polynucleotide encoding luciferase (Example 4), the expression of which can be detected in vivo or in vitro, and by polynucleotides encoding antibodies (Example 3). Furthermore, the exemplified compositions and methods demonstrate that functional fusion proteins can be expressed robustly in chloroplasts, including single chain antibodies and reporter polypeptides (see Examples 3 and 4).

The chloroplasts of higher plants and algae likely originated by an endosymbiotic incorporation of a photosynthetic prokaryote into a eukaryotic host. During the integration process genes were transferred from the chloroplast to the host nucleus (Gray, Curr. Opin. Gen. Devel. 9:678-687, 1999). As such, proper photosynthetic function in the chloroplast requires both nuclear encoded proteins and plastid encoded proteins, as well as coordination of gene expression between the two genomes. Expression of nuclear and chloroplast encoded genes in plants is acutely coordinated in response to developmental and environmental factors.

In chloroplasts, regulation of gene expression generally occurs after transcription, and often during translation initiation. This regulation is dependent upon the chloroplast translational apparatus, as well as nuclear-encoded regulatory factors (see Barkan and Goldschmidt-Clermont, Biochemie 82:559-572, 2000; Zerges, Biochemie 82:583-601, 2000; Bruick and Mayfield, supra, 1999). The chloroplast translational apparatus generally resembles that in bacteria; chloroplasts contain 70S ribosomes; have mRNAs that lack 5′ caps and generally do not contain 3′ poly-adenylated tails (Harris et al., Microbiol. Rev. 58:700-754, 1994); and translation is inhibited in chloroplasts and in bacteria by selective agents such as chloramphenicol.

In bacteria, the RNA elements that mediate proper translation initiation include an initiation codon, an RBS, a defined spacing between the RBS and the initiation codon, translational enhancer sequences, bias at the second codon, and secondary structures that affect RNA accessibility (Gold, Ann. Rev. Biochem. 57:199-233, 1988). In chloroplasts, ribosome binding and proper translation start site selection are mediated, at least in part, by cis-acting RNA elements (see Bruick and Mayfield, supra, 1999). Like bacteria, chloroplast initiation codons affect the efficiency of translation initiation, but do not determine the location of the initiation site (Chen et al., Plant Cell 7:1295-1305, 1995), indicating that additional determinants are required for translation start site selection in chloroplasts.

Several RNA elements that act as mediators of translational regulation have been identified within the 5′UTR's of chloroplast mRNAs (Alexander et al., Nucl. Acids Res. 26:2265-2272, 1998; Hirose and Sugiura, EMBO J. 15:1687-1695, 1996; Mayfield et al., J. Cell Biol. 127:1537-1545, 1994; Sakamoto et al., Plant J. 6:503-512, 1994; Zerges et al., supra, 1997, each of which is incorporated herein by reference). These elements may interact with nuclear-encoded factors and generally do not resemble known prokaryotic regulatory sequences (McCarthy and Brimacombe, Trends Genet. 10:402-407, 1994).

Consensus prokaryotic RBS elements feature a Shine-Dalgarno (SD) sequence, which is a sequence containing three to nine nucleotides, including generally about 4, 5 or 6 nucleotides that are complementary to the 3′ end of the 16S rRNA. Early in translation initiation, the 30S ribosomal subunit binds the mRNA at the SD sequence by virtue of the complementary anti-SD sequence within the 16S rRNA. Because the SD sequence in prokaryote mRNAs is located 5 to 15 nucleotides upstream of the initiation codon, the 30S ribosomal subunit is positioned such that the proper initiation codon resides within the ribosomal P site.

Many chloroplast mRNAs contain elements resembling prokaryotic RBS elements (Bonham-Smith and Bourque, Nucl. Acids Res. 17:2057-2080, 1989; Ruf and Kossel, FEBS Lett. 240:41-44, 1988, each of which is incorporated herein by reference). However, the functional utility of these RBS sequences in chloroplast translation has been unclear because these elements are often located further upstream of the start codon than is typically observed in prokaryotes. In some studies, alteration of a putative RBS in the 5′UTR's of chloroplast mRNAs was reported to affect translation (Betts and Spremulli, J. Biol. Chem. 269:26456-26465, 1994; Hirose et al., FEBS Lett. 430:257-260, 1998; Hirose and Sugiura, supra, 1996; Mayfield et al., supra, 1994), whereas alteration of potential RBS elements in other chloroplast mRNAs had little affect on translation (Fargo et al., Mol. Gen. Genet. 257:271-282, 1998; Koo and Spremulli, J. Biol. Chem. 269:7494-7500, 1994; Rochaix, Plant Mol. Biol. 32:327-341, 1996; Sakamoto et al., supra, 1994). Interpretation of these results has been complicated by the lack of a consensus for chloroplast RBS elements, and because the mutations generated to study these putative RBS sequences may have altered the context of other important sequences within the 5′UTR.

A functional role for RBS elements in chloroplast translation is disclosed herein (Example 2). Mutations to the chloroplast 16S rRNA anti-SD sequence, which is positioned at the 3′ end of the 16S rRNA, and has the sequence 3′-CUUCCUCCAC-5′ (SEQ ID NO:29), that eliminated potential base pairing with the SD sequence of chloroplast mRNAs severely impaired translation of several chloroplast-encoded integral membrane proteins in C. reinhardtii (Example 2). Ribosomes bearing the 16S rRNA anti-SD mutations remained competent for translation, as the synthesis of soluble chloroplast proteins was largely unaffected by these mutations.

Analysis of potential SD elements in the 5′UTR of the chloroplast psbA mRNA, encoding the photosystem II reaction center D1 protein, revealed the presence of a single prokaryotic-like RBS element positioned 27 nucleotides 5′ (upstream) of the initiator AUG codon. This RBS is too far upstream of the start codon to allow the 30S ribosomal subunit to simultaneously contact both the RBS element and the initiation codon, as in bacteria. When the RBS was repositioned closer to the start codon, it no longer supported translation initiation in the chloroplast, but rendered the transcripts newly competent for translation in E. coli (Example 2). Because a pre-initiation complex can form at this RBS element, it has the characteristics of a bonafide recognition site for the 30S ribosomal subunit. However, the RBS element is unable to correctly define the translational start site in the absence of additional factors, which include nuclear-encoded translational activator proteins (Danon and Mayfield, 1991; Yohn et al., 1998a; Yohn et al., 1998b). This result indicates that the additional distance between the RBS and the initiation codon in the psbA mRNA accommodates additional translation factors, as exemplified by function of the RBS elements in chloroplasts to promote translation initiation in conjunction with light-regulated trans-acting factors.

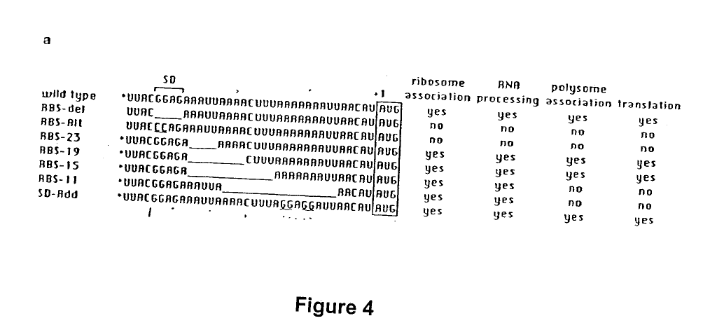

Accordingly, the invention provides an isolated ribonucleotide sequence that includes a first RBS operatively linked to a second RBS. As disclosed herein, such operatively linked first and second RBS generally are spaced apart by about 5 to 25 nucleotides such that, when the ribonucleotide sequence is operatively linked to a polynucleotide encoding a polypeptide, the first RBS can direct translation of the polypeptide in a prokaryote and the second RBS can direct translation of the polypeptide in a chloroplast. An RBS is active in translation in chloroplasts, including allowing polysome formation, when it is positioned at least about 19 nucleotides upstream (5′) of the initiator AUG codon, whereas positioning the RBS closer to the AUG results a loss of translation activity in chloroplasts (see FIG. 4). As shown in FIG. 4, the RBS (SD sequence) of the psbA mRNA begins at position −27 (i.e., following position −27 upstream of the AUG codon). Deletions bringing the RBS closer than about 19 nucleotides to the AUG codon resulted in a substantial loss of translation and polysome formation in chloroplasts, but resulted in an increased translational activity in bacteria (see Example 2, also showing decreased translational activity in bacteria for RBS greater than about 15 nucleotides from the AUG codon).

An isolated ribonucleotide sequence of the invention generally is about 11 to 50 nucleotides in length, and can be about 15 to 40 nucleotides in length or about 20 to 30 nucleotides. Such a length allows for two SD sequences, which generally are about 3 to 9 nucleotides in length, usually about 4 to 7 nucleotides in length, to be spaced apart by about 5 to 25 nucleotides (generally by about 10 to 20 nucleotides, and particularly by about 15 nucleotides). For example, a ribonucleotide sequence of the invention can include a first RBS of 4 nucleotides, e.g., GGAG, spaced apart by 5 nucleotides from a second of about 4 nucleotides, e.g., GGAG, thus providing a ribonucleotide sequence of 13 nucleotides in length. Each of the first RBS and the second RBS independently can have any sequence characteristic of a SD sequence. As disclosed herein, an RBS useful for directing translation in a plant chloroplast is complementary to at least three, particularly, four, five, or six, or more, of the anti-SD sequence at the 3′ end of 16S rRNA (3′-CUUCCUCCAC-5′; SEQ ID NO:29), particularly complementary to the central eight nucleotides of the anti-SD sequence. For example, RBS sequences comprising GGAG, GGAGG, or ACGAGA (nucleotides complementary to SEQ ID NO:29 in italics) directed translation in plant chloroplasts, when operatively linked to an encoded polypeptide.

An RBS useful in preparing a composition of the invention or in practicing a method of the invention can be chemically synthesized, or can be isolated from a naturally occurring nucleic acid molecule. For example, an RBS that directs translation in a chloroplast generally is present in the 5′UTR of a chloroplast gene and, therefore, can be isolated from a chloroplast gene. In addition, there can be advantages to including additional nucleotide sequences as are normally associated with the SD sequence in the gene. For example, a 5′UTR can include transcriptional regulatory elements such as a promoter, thus facilitating construction of a recombinant nucleic acid molecule that can be transcribed and translated in a plant chloroplast. In addition, as disclosed herein, the inclusion of additional 5′UTR sequences from a chloroplast gene encoding the membrane associated D1 (psbA) chloroplast protein resulted in expression of a membrane heterologous polypeptide in the chloroplasts (Example 3). As such, a ribonucleotide of the invention containing an RBS that directs translation in a chloroplast, can further contain a 5′UTR of a chloroplast gene, for example, a 5′UTR of a chloroplast gene that encodes a soluble protein, or a 5′UTR of a gene encoding a membrane-bound chloroplast protein. Such 5′UTRs are well known in the art and include those encoded by chloroplast genes encoding soluble proteins, for example, an AtpA 5′UTR (SEQ ID NO:4) or a RbcL 5′UTR (SEQ ID NO:5), and those encoded by chloroplast genes encoding membrane bound proteins, for example, a PsbD 5′UTR (SEQ ID NO:6), or a PsbA 5′UTR (SEQ ID NO:7). In addition, a 16S rRNA 5′UTR (SEQ ID NO:8) can be used, for example, to direct transcription of an operatively linked heterologous polynucleotide, and can be modified at the sequence complementary to the anti-SD sequence to generate an RBS that is particularly useful for directing translation of a polypeptide encoded by the polynucleotide in plant chloroplasts.

A ribonucleotide sequence of the invention can further include an initiation codon, for example, an initiator AUG codon, operatively linked to the first and second RBS. Such an initiator AUG codon can further include adjacent nucleotides of a Kozak sequence, for example, ACCAUGG or GCCAUGG or CC(A/G)CCAUGG or the like (see Kozak, J. Mol. Biol. 196:947-950, 1987, which is incorporated herein by reference), which can facilitate translation of an encoded polypeptide in a cell. In addition, the ribonucleotide sequence of the invention can be operatively linked to a polynucleotide encoding a polypeptide, wherein the polynucleotide contains an initiation codon, which can, but need not, be an endogenous initiation codon, or can be modified to contain an initiation codon.

An isolated ribonucleotide sequence of the invention can be chemically synthesized, or can be generated using an enzymatic method, for example, from a DNA or RNA template using a DNA dependent RNA polymerase or an RNA dependent RNA polymerase, respectively. A DNA template encoding the ribonucleotide of the invention can be chemically synthesized, can be isolated from a naturally occurring DNA molecule, or can be derived from a naturally occurring DNA sequence that is modified to have the required characteristics. For example, a DNA sequence of a prokaryote gene normally has nucleotide sequence encoding an RBS positioned about 5 to 15 nucleotides upstream an initiation codon. Such a nucleotide sequence can be isolated and modified using routine recombinant DNA methods to contain a second RBS appropriately position upstream (5′) of the endogenous prokaryote RBS. Accordingly, the present invention provides a polynucleotide encoding an operatively linked first RBS and second RBS as defined herein.

A polynucleotide encoding a first RBS operatively linked to a second RBS, wherein the first RBS can direct translation in a prokaryote and the second RBS can direct translation in a chloroplast, can be DNA or RNA, and can be single stranded or double stranded. The polynucleotide also can include an initiation codon, e.g., ATG, operatively linked to the nucleotide sequence encoding the first RBS and second RBS, i.e., an ATG codon positioned about 3 to 15 nucleotides, including about 4, 5, 6, 7, 8, 9, 10, 11, 12, 13 or 14 nucleotides, downstream (3′) of the first RBS, which directs translation in a prokaryote. A polynucleotide of the invention also can include a cloning site that is positioned to allow operative linkage of an expressible polynucleotide, which can encode a polypeptide, to the first RBS and second RBS, and to an ATG codon if present, such that the polypeptide can be expressed in a chloroplast or in a prokaryote host cell.

As used herein, the term “cloning site” is used broadly to refer to any nucleotide or nucleotide sequence that facilitates linkage of a first polynucleotide to a second polynucleotide. Generally, a cloning site comprises one or a plurality restriction endonuclease recognition sites, for example, a multiple cloning site, or one or a plurality of recombinase recognition sites, for example, a loxP site or an att site, or a combination of such sites. The cloning site can be provided to facilitate insertion or linkage, which can be operative linkage, of the first and second polynucleotide, for example, a first polynucleotide encoding a first RBS operatively linked to a second RBS to a second polynucleotide encoding a polypeptide of interest, which is to be translated in a prokaryote or a chloroplast or both.

A polynucleotide encoding a first and second RBS, as defined herein, can be operatively linked to an expressible polynucleotide, which can encode at least one polypeptide, including a peptide or peptide portion of a polypeptide. As such, the expressible polynucleotide can encode only a first polypeptide, or can encode two or more polypeptides, which can be the same or different as the first polypeptide. For example, the expressible polynucleotide can encode a first polypeptide and a second polypeptide, which are different from each other, particularly a first and second polypeptide that can specifically associate to form a functional heterodimer such as an antibody; an enzyme; a cell surface receptor such as a T cell receptor, a growth factor receptor, a cannabinoid receptor; or the like. Such a first and second (or other) polypeptide can be expressed as a fusion protein, for example, single chain antibody comprising a H chain linked to a L chain, or can be expressed as separate and discrete polypeptides, which can, but need not, have the ability to specifically associate to form a functional protein complex. Where the polypeptides are to be expressed as separate entities, it can be useful to include a nucleotide sequence encoding an internal ribosome entry site (IRES) operatively linked between the coding sequence of the first polypeptide and the coding sequence of the second polypeptide, thus facilitating translation of the second (or downstream) polypeptide.

A polynucleotide encoding a first RBS operatively linked to a second RBS, as defined herein, can be a linear nucleotide sequence, and can be flanked at one end by a first cloning site and the second end by a second cloning site, thus providing a cassette that readily can be inserted into or linked to a second polynucleotide. The flanking first and second cloning sites can be the same or different, and one or both independently can comprise a multiple cloning site. The polynucleotide can further include any other nucleotide sequences of interest, for example, an operatively linked initiator ATG codon.

The present invention further provides a vector containing a polynucleotide encoding an first RBS operatively linked to a second RBS, as defined herein. The vector can be any vector useful for introducing a polynucleotide into a prokaryotic or eukaryotic cell, including a cloning vector or an expression vector. In one embodiment, the vector comprises a nucleotide sequence of chloroplast genomic DNA sufficient to undergo homologous recombination with chloroplast genomic DNA, particularly a silent nucleotide sequence, which does not encode a chloroplast gene. Such chloroplast vectors are well known in the art and include, for example, p322 (see Example 1; see, also, Kindle et al., Proc. Natl. Acad. Sci., USA 88:1721-1725, 1991, which is incorporated herein by reference; Hager and Bock, supra, 2000; Bock, supra, 2001).

A vector of the invention also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, sequences that encode a selectable marker, and the like. As such, the vector can contain, for example, one or more cloning sites such as a multiple cloning site, which can, but need not, be positioned such that a heterologous polynucleotide can be inserted into the vector and operatively linked to the first RBS and second RBS. The vector also can contain a prokaryote origin of replication (ori), for example, an E. coli ori or a cosmid ori, thus allowing passage of the vector in a prokaryote host cell, as well as in a plant chloroplast, as desired.

The term “regulatory element” is used broadly herein to refer to a nucleotide sequence that regulates the transcription or translation of a polynucleotide or the localization of a polypeptide to which it is operatively linked. In addition to an RBS, an expression control sequence can be a promoter, enhancer, transcription terminator, an initiation (start) codon, a splicing signal for intron excision and maintenance of a correct reading frame, a STOP codon, an amber or ochre codon, an IRES, or a sequence that targets a polypeptide to a particular location, for example, a cell compartmentalization signal, which can be useful for targeting a polypeptide to the cytosol, nucleus, plasma membrane, endoplasmic reticulum, mitochondrial membrane or matrix, chloroplast membrane or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see, also, Hancock et al., EMBO J 10:4033-4039, 1991; Buss et al., Mol. Cell. Biol. 8:3960-3963, 1988; U.S. Pat. No. 5,776,689, each of which is incorporated herein by reference). Inclusion of a cell compartmentalization domain in a polypeptide produced using a method of the invention can allow use of the polypeptide, which can comprise a protein complex, where it is desired to target the polypeptide to a particular cellular compartment in an individual.

A vector or other recombinant nucleic acid molecule of the invention can include a nucleotide sequence encoding a reporter polypeptide or other selectable marker. The term “reporter” or selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells or plant cells or both that express the marker and, therefore, can be useful as a component of a vector of the invention (see, for example, Bock, supra, 2001). Examples of selectable markers include those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983), hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39). Since a composition or a method of the invention can result in expression of a polypeptide in chloroplasts, it can be useful if a polypeptide conferring a selective advantage to a plant cell is operatively linked to a nucleotide sequence encoding a cellular localization motif such that the polypeptide is translocated to the cytosol, nucleus, or other subcellular organelle where, for example, a toxic effect due to the selectable marker is manifest (see, for example, Von Heijne et al., Plant Mol. Biol. Rep. 9: 104, 1991; Clark et al., J. Biol. Chem. 264:17544, 1989; della Cioppa et al., Plant Physiol. 84:965, 1987; Romer et al., Biochem. Biophys. Res. Comm. 196:1414, 1993; Shah et al., Science 233:478, 1986; Archer et al., J. Bioenerg Biomemb. 22:789, 1990; Scandalios, Prog. Clin. Biol. Res. 344:515, 1990; Weisbeek et al., J. Cell Sci. Suppl. 11: 199, 1989; Bruce, Trends Cell Biol. 10:440, 2000.

The ability to passage a shuttle vector of the invention in a prokaryote allows for conveniently manipulating the vector. For example, a reaction mixture containing the vector and putative inserted polynucleotides of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. The shuttle vector then can be introduced into plant cell chloroplasts, wherein a polypeptide of interest can be expressed and, if desired, isolated according to a method of the invention.

A polynucleotide or recombinant nucleic acid molecule of the invention, which can be contained in a vector, including a vector of the invention, can be introduced into plant chloroplasts using any method known in the art. As used herein, the term “introducing” means transferring a polynucleotide into a cell, including a prokaryote or a plant cell, particularly a plant cell plastid. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, or the “glass bead method” (see, for example, Kindle et al., supra, 1991), or by pollen-mediated transformation, liposome-mediated transformation, transformation using wounded or enzyme-degraded immature embryos, or wounded or enzyme-degraded embryogenic callus (see Potrykus, Ann. Rev. Plant. Physiol. Plant Mol. Biol. 42:205-225, 1991, which is incorporated herein by reference).

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994, each of which is incorporated herein by reference). Chloroplast transformation involves introducing regions of chloroplast DNA flanking a desired nucleotide sequence into a suitable target tissue; using, for example, a biolistic or protoplast transformation method (e.g., calcium chloride or PEG mediated transformation). One to 1.5 kb flanking nucleotide sequences of chloroplast genomic DNA allow homologous recombination of the vector with the chloroplast genome, and allow the replacement or modification of specific regions of the plastome. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990; Staub and Maliga, supra, 1992), and can result in stable homoplasmic transformants, at a frequency of approximately one per 100 bombardments of target leaves. The presence of cloning sites between these markers provides a convenient nucleotide sequence for making a chloroplast vector (Staub and Maliga, EMBO J. 12:601-606, 1993), including a vector of the invention. Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga, Proc. Natl. Acad. Sci., USA 90:913-917, 1993). Approximately 15 to 20 cell division cycles following transformation are generally required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

A direct gene transfer method such as electroporation also can be used to introduce a polynucleotide of the invention into a plant protoplast (Fromm et al., Proc. Natl. Acad. Sci., USA 82:5824, 1985, which is incorporated herein by reference). Electrical impulses of high field strength reversibly permeabilize membranes allowing the introduction of the polynucleotide. Electroporated plant protoplasts reform the cell wall, divide and form a plant callus. Microinjection can be performed as described in Potrykus and Spangenberg (eds.), Gene Transfer To Plants (Springer Verlag, Berlin, NY 1995). A transformed plant cell containing the introduced polynucleotide can be identified by detecting a phenotype due to the introduced polynucleotide, for example, expression of a reporter gene or a selectable marker.

Microprojectile mediated transformation also can be used to introduce a polynucleotide into a plant cell chloroplast (Klein et al., Nature 327:70-73, 1987, which is incorporated herein by reference). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known (Wan, Plant Physiol. 104:37-48, 1984; Vasil, BioTechnology 11: 1553-1558, 1993; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method (Kindle et al., supra, 1991), and the like.

The present invention also provides a vector that includes a nucleotide sequence encoding an RBS positioned about 20 to 40 nucleotides 5′ to a cloning site. The cloning site can be any nucleotide sequence that facilitates insertion or linkage of a heterologous nucleotide sequence into the vector, for example, one or more restriction endonuclease recognition sites, one or more recombinase recognition sites, or a combination of such sites. Preferably, the cloning site is a multiple cloning site, which includes a plurality of restriction endonuclease recognition sites or recombinase recognition sites, or a combination of at least one restriction endonuclease recognition site and at least one recombinase recognition site. The vector can further contain an initiation codon or a portion thereof adjacent and 5′ to the cloning site, thus providing a translation start site (or cryptic start site) for a coding sequence that otherwise lacks an initiator ATG codon or contains a partial initiation codon due, for example, to cleavage by a restriction endonuclease. The vector also can contain a chloroplast gene 3′UTR positioned 3′ to the cloning site, for example, PsbA 3′UTR (SEQ ID NO:9), a RbcL 3′UTR (SEQ ID NO:10), an AtpA 3′UTR (SEQ ID NO: 11), a tRNA^{ARG} 3′UTR (SEQ ID NO: 12), or a PsbD 3′UTR (see SEQ ID NO:30, beginning at position 1553; also showing insertion site for GFP construct encoding PsbD-GFP fusion protein).

Also provided is a method of making a chloroplast/prokaryote shuttle expression vector. A shuttle vector of the invention can be made, for example, by introducing into a nucleotide sequence of chloroplast genomic DNA sufficient to undergo homologous recombination with chloroplast genomic DNA, a nucleotide sequence comprising a prokaryote origin of replication; a nucleotide sequence encoding a first RBS; and a nucleotide sequence encoding a second RBS, wherein the first RBS and second RBS are spaced apart by about 5 to 25 nucleotides; and a cloning site, wherein the cloning site is positioned to allow operative linkage of a polynucleotide encoding a polypeptide to the first RBS and second RBS such that the first RBS can direct translation of the polypeptide in a prokaryote and the second RBS can direct translation of the polypeptide in a chloroplast. A method of making a chloroplast/prokaryote shuttle expression vector also can be performed by genetically modifying a nucleotide sequence of chloroplast genomic DNA, which is sufficient to undergo homologous recombination with chloroplast genomic DNA, to contain a prokaryote origin of replication, a nucleotide sequence encoding a first RBS spaced apart from a second RBS by about 5 to 25 nucleotides, and a cloning site positioned to allow operative linkage of a polynucleotide encoding a polypeptide to the first RBS and second RBS such that the first RBS can direct translation of the polypeptide in a prokaryote and the second RBS can direct translation of the polypeptide in a chloroplast. Accordingly, the present invention also provides a chloroplast/prokaryote shuttle vector produced by a method as disclosed herein.

The invention also provides a recombinant nucleic acid molecule, which includes a first nucleotide sequence encoding chloroplast RBS operatively linked to a second nucleotide sequence encoding a polypeptide, wherein the first nucleotide sequence is heterologous with respect to the second nucleotide sequence. An operatively linked RBS generally is positioned about 20 to 40 nucleotides 5′ (upstream) to an initiation codon, which, in turn, is operatively linked to the nucleotide sequence encoding the polypeptide. In one embodiment, the first nucleotide sequence comprises an ATG codon positioned about 20 to 40 nucleotides 3′ of nucleotide sequence encoding the RBS. A recombinant nucleic acid molecule of the invention can further include other regulatory elements or encoding polynucleotides of interest, as exemplified herein or otherwise known in the art.

Reporter genes have been successfully used in chloroplasts of higher plants, and high levels of recombinant protein expression have been reported. In addition, reporter genes have been used in the chloroplast of C. reinhardtii, but, in most cases very low amounts of protein were produced. Reporter genes greatly enhance the ability to monitor gene expression in a number of biological organisms. In chloroplasts of higher plants, β-glucuronidase (uidA, Staub and Maliga, EMBO J. 12:601-606, 1993), neomycin phosphotransferase (nptII, Carrer et al., Mol. Gen. Genet. 241:49-56, 1993), adenosyl-3-adenyltransferase (aadA, Svab and Maliga, Proc. Natl. Acad. Sci., USA 90:913-917, 1993), and the Aequorea victoria GFP (Sidorov et al., Plant J. 19:209-216, 1999) have been used as reporter genes (Heifetz, Biochemie 82:655-666, 2000). Each of these genes has attributes that make them useful reporters of chloroplast gene expression, such as ease of analysis, sensitivity, or the ability to examine expression in situ. Based upon these studies, other heterologous proteins have been expressed in the chloroplasts of higher plants such as Bacillus thuringiensis Cry toxins, conferring resistance to insect herbivores (Kota et al., Proc. Natl. Acad. Sci., USA 96:1840-1845, 1999), or human somatotropin (Staub et al., Nat. Biotechnol. 18:333-338, 2000), a potential biopharmaceutical.

Several reporter genes have been expressed in the chloroplast of the eukaryotic green alga, C. reinhardtii, although with varying degrees of success. These include aadA (Goldschmidt-Clermont, Nucl. Acids Res. 19:4083-4089 1991; Zerges and Rochaix, Mol. Cell Biol. 14:5268-5277, 1994), uidA (Sakamoto et al., Proc. Natl. Acad. Sci., USA 90:477-501, 19933, Ishikura et al., J. Biosci. Bioeng. 87:307-314 1999), Renilla luciferase (Minko et al., Mol. Gen. Genet. 262:421-425, 1999) and the amino glycoside phosphotransferase from Acinetobacter baumanii, aphA6 (Bateman and Purton, Mol. Gen. Genet 263:404-410, 2000). The amount of recombinant protein produced was reported for the uidA gene only (Ishikura et al., supra, 1999) and, and based on western blot analysis and activity measurements, very low amounts were produced. In order to improve expression of heterologous polypeptides in chloroplasts, the effect of codon bias described for the C. reinhardtii chloroplast genome (Nakamura et al., supra, 1999), was examined.

Due to the redundancy inherent in the genetic code, up to six nucleotide triplets can encode the same amino acid, and iso-accepting tRNAs are often encoded by multigene families. In Caenorhabditis elegans, for which the entire complement of nuclear tRNA genes is known, there are 31 tRNA_{UCC}^{Gly} encoding genes, for example (Duret, Trends Genet. 16:287-289, 2000). A consequence of this redundancy is that many organisms display a clear codon bias, wherein certain codons are used more frequently than others. The effect of codon bias on heterologous protein expression is well documented in both prokaryotic and eukaryotic organisms, and even viral genes display a codon bias that can affect their temporal, and tissue specific expression. Typically, codon usage is correlated with the level of iso-accepting tRNAs. As such, genes encoding highly expressed proteins tend to utilize codons whose levels of cognate tRNAs are particularly abundant (Duret, supra, 2000; Kanaya et al., Gene 238:143-155, 1999).

The C. reinhardtii chloroplast genome displays a strong codon bias, with adenine or uracil (or thymine) preferred at the third position (Nakamura et al., supra, 1999). The role of chloroplast codon usage in expression of recombinant polypeptides in the C. reinhardtii chloroplasts was examined by synthesizing de novo a polynucleotide that encodes GFP and is biased for chloroplast codon usage of the major C. reinhardtii chloroplast encoded proteins (Example 1). GFP accumulation was monitored in C. reinhardtii chloroplasts transformed with the codon optimized GFP cassette (GFPct; SEQ ID NO: 1) under the control of the C. reinhardtii RbcL 5′ UTR and 3′ UTR (SEQ ID NOS:5 and 10, respectively), and compared to the accumulation of GFP in C. reinhardtii transformed with a non-optimized GFP cassette (GFPncb; SEQ ID NO:3). As disclosed herein, C. reinhardtii chloroplasts transformed with the GFPct cassette accumulated approximately 80 fold more GFP than GFPncb transformed strains, and expression was sufficiently robust to report differences in protein synthesis based upon subtle changes in environmental conditions (Example 1). Similar results were obtained for luciferase, wherein expression of a chloroplast codon biased synthetic polynucleotide (SEQ ID NO:45) encoding a fusion luciferase protein comprising the bacterial luciferase A subunit fused via a peptide linker to the bacterial luciferase B subunit (SEQ ID NO:46) resulted in robust expression of luciferase, and provided the additional advantage that the luciferase expression could be detected in vivo (see Example 4).

Accordingly, the present invention provides an isolated synthetic polynucleotide encoding a fluorescent protein or a mutant or variant thereof, wherein codons of the polynucleotide are biased to reflect chloroplast codon usage. The synthetic polynucleotide can be DNA or RNA, can be single stranded or double stranded, and can be a linear polynucleotide containing a cloning site at one or both ends. The polynucleotide, which can be contained in a vector, also can be operatively linked to a polynucleotide encoding a first RBS and a second RBS that are spaced apart by about 5 to 25 nucleotides, such that the fluorescent protein conveniently can be translated in a prokaryote and in a chloroplast.

Table 1 exemplifies codons that are preferentially used in alga chloroplast genes. The term “chloroplast codon usage” is used herein to refer to such codons, and is used in a comparative sense with respect to degenerate codons that encode the same amino acid but are less likely to be found as a codon in a chloroplast gene. The term “biased”, when used in reference to chloroplast codon usage, refers to the manipulation of a polynucleotide such that one or more nucleotides of one or more codons is changed, resulting in a codon that is preferentially used in chloroplasts. Chloroplast codon bias is exemplified herein by the alga chloroplast codon bias as set forth in Table 1. The chloroplast codon bias can, but need not, be selected based on a particular plant in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast codon bias as a means to provide efficient translation of a polypeptide, it will be recognized that an alternative means for obtaining efficient translation of a polypeptide in a chloroplast to re-engineer the chloroplast genome (e.g., a C. reinhardtii chloroplast genome) for the expression of tRNAs not otherwise expressed by in the chloroplast genome. Such an engineered algae expressing one or more heterologous tRNA molecules provides the advantage that it would obviate a requirement to modify every polynucleotide of interest that is to be introduced into and expressed from a chloroplast genome; instead, algae such as C. reinhardtii that comprise a genetically modified chloroplast genome can be provided and utilized for efficient translation of a polypeptide according to a method of the invention. Correlations between tRNA abundance and codon usage in highly expressed genes is well known (Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; Komar et. al., Biol. Chem. 379:1295-1300, 1998, each of which is incorporated herein by reference. In E. coli, for example, re-engineering of strains to express underutilized tRNAs resulted in enhanced expression of genes which utilize these codons (see Novy et al., in Novations 12:1-3, 2001, which is incorporated herein by reference). Utilizing endogenous tRNA genes, site directed mutagenesis can be used to make a synthetic tRNA gene, which can be introduced into chloroplasts to complement rare or unused tRNA genes in a chloroplast genome such as a C. reinhardtii chloroplast genome.

One or more codons encoding a fluorescent protein of the invention can be biased, for example, to contain an adenine or a thymine at position three, thus facilitating translation of the fluorescent protein in a chloroplast. As disclosed herein, the polynucleotide encoding Aequorea victoria GFP was biased by de novo synthesis of an encoding sequence having 121 synonymous codon changes, including 66 changes that represent a modest shift toward chloroplast codon usage and 54 changes that resulted in an infrequently used codon being shifted toward chloroplast codon usage (Example 1). As such, the polynucleotide set forth as SEQ ID NO: 1, which encodes a modified GFP (SEQ ID NO:2), provides an example of a polynucleotide of the invention, and polynucleotides that encode SEQ ID NO:2 but have fewer biased codons provide additional examples. Also provided is the modified GFP having an amino acid sequence as set forth in SEQ ID NO:2.

GFPs are well known in the art and have been isolated from the Pacific Northwest jellyfish, Aequorea victoria, the sea pansy, Renilla reniformis, and Phialidium gregarium (Ward et al., Photochem. Photobiol. 35:803-808, 1982; Levine et al., Comp. Biochem. Physiol. 72B:77-85, 1982, each of which is incorporated herein by reference). Similarly, red fluorescent proteins are known and have been isolated from the coral, Discosoma (Matz et al., Nature Biotechnol. 17:969-973, 1999, which is incorporated herein by reference). In addition, a variety of Aequorea GFP-related fluorescent proteins having useful excitation and emission spectra have been engineered by modifying the amino acid sequence of a naturally occurring GFP from A. Victoria (see Prasher et al., Gene 111:229-233, 1992; Heim et al., Proc. Natl. Acad. Sci., USA 91:12501-12504, 1994; U.S. Pat. No. 6,319,669; Intl. Appl. No. PCT/US95/14692, each of which is incorporated herein by reference). As such, it will be recognized that the nucleotide sequences encoding such fluorescent proteins can be biased for chloroplast codon usage and, therefore, provide additional examples of fluorescent proteins of the invention.

The following examples are intended to illustrate but not limit the invention.

### EXAMPLE 1

### Optimization of a Polypeptide Coding Sequence for Expression in Chloroplasts

This example demonstrates that an chloroplast codon biased nucleotide sequence encoding green fluorescent protein is efficiently expressed in alga chloroplasts (see, also, Franklin et al., Plant J. 30:733-744, 2002, which is incorporated herein by reference). C. reinhardtii strains, transformation and growth conditions

All transformations were carried out on C. reinhardtii strain 137c (mt+). Cells were grown to late log phase (approximately 7 days) in the presence of 40 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 4° C. for 5 min. The supernatant was decanted and cells resuspended in 4 ml TAP medium for subsequent chloroplast transformation by particle bombardment (Cohen et al., supra, 1998). All transformations were carried out under spectinomycin selection (150 μg/ml), in which resistance was conferred by co-transformation with the spectinomycin resistance ribosomal gene of plasmid p228 (Chlamydomonas Stock Center, Duke University).

Cultivation of C. reinhardtii transformants for expression of GFP was carried out in TAP medium (Gorman and Levine, supra, 1965) at 23° C. under constant illumination of 5,000 Lux on a rotary shaker set at 100 rpm, unless stated otherwise. Cultures were maintained at a density of 1×10⁷ cells per ml for at least 48 hr prior to harvest.

Plasmid Construction

All DNA and RNA manipulations were carried out essentially as described by Sambrook et al., supra, 1989, and Cohen et al., supra, 1998. The coding region of the GFP gene was amplified via PCR from a plasmid containing the native GFP (GFPncb) sequence (Tsien, Ann. Rev. Biochem. 67:509-544, 1998, which is incorporated herein by reference). PCR primers were designed to generate a 5′ Nde I site and a 3′ Xba I site immediately outside the coding region, to facilitate subsequent cloning. The sequence for the 5′ GFPncb was 5′-CATATGAGTAAAGGAGAAGAAC-3′ (SEQ ID NO:17); the sequence for the 3′ GFPct primer was 5′-TCTAGATTATTTGTATAGTTCATCC-3′ (SEQ ID NO: 18). The coding region of the GFPct gene was synthesized de novo as described by Stemmer et al., Gene 164:49-53, 1995, which is incorporated herein by reference) from a pool of primers, each 40 nucleotides in length. The 5′ terminal and 3′ terminal primers contained restriction sites for Nde I and Xba I, respectively.

The resulting 717 bp PCR products containing the GFPct and GFPncb genes were cloned into plasmid pCR2.1 TOPO (Invitrogen, Inc.) according to the manufacturers protocol to generate plasmids pCrGFPct and pCrGFPncb respectively. The rbcL 3′ UTR was generated via PCR using a 1.6 kb Hind III fragment of C. reinhardtii chloroplast genomic DNA, cloned into plasmid pUC 19, as the template. The sequence of the PCR primer, corresponding to the 5′ end of the rbcL 3′ UTR and a portion of the pUC19 polylinker, including the Xba I site was 5′-TCTAGAGTCGACCTGCAG-3′ (SEQ ID NO: 19). The sequence of the PCR primer, corresponding to the 3′ end of the rbcL 3′ UTR was 5′-GGATCCGTCGACGTATG-3′ (SEQ ID NO:20), and includes a Bam HI restriction site for subsequent cloning. The resulting 433 bp product was cloned into plasmid pCR2.1 TOPO to generate plasmid p3rbcL.

The rbcL 5′ UTR was generated by PCR using C. reinhardtii genomic DNA as template. The sequence of the PCR primer, complementary to the 5′ end of the rbcL gene beginning at position −189 relative to the translational start site was 5′-GAATTCATATACCTAAAGGCCCTTTCTATGC-3′ (SEQ ID NO:21), and contains an Eco RI restriction site. The PCR primer complementary to the 3′ end of the rbcL 5′UTR begins at the translation initiation site and had the sequence 5′-CATATGTATAAATAAATGTAACTTC-3′ (SEQ ID NO:22), and contains a Nde I restriction site. The resulting 241 bp PCR product was cloned into the pCR2.1 TOPO vector to generate plasmid p5rbcL.

The plasmid p5rbcL was digested with Bam HI and Nde I and the resulting fragment was ligated into either pCrGFPct or pCrGFPncb digested with Bam HI and Nde I to generate plasmids p5CrGFPct and p5CrGFPncb respectively. Finally, p5CrGFPct and p5CrGFPncb were digested with Bam HI and Xba I and the resulting 958 bp fragments were ligated into p3rbcL, also digested with Barn HI and Xba I, to generate plasmids p53rGFPct and p53rGFPncb.

Both p53rGFPct and p53rGFPncb were digested with Nde I and Bam HI and the 1.2 kb fragments were ligated into pET19b (Novagen) to generate plasmids pETGFPct and pETGFPncb, respectively, for expression in E. coli. p53rGFPct and p53rGFPncb were next digested with Bam HI and the 1.43 kb fragments were ligated into the C. reinhardtii chloroplast transformation vector, p322 (Chiamydomonas Genetics Center, Duke University) to form plasmids pExGFPct and pExGFPncb.

The p322 vector is based on the nucleotide sequence of the C. reinhardtii chloroplast genomic DNA sequence extending from the Eco (Eco RI) site beginning at position 143,073 to the Xho (Xho I) site beginning at position 148,561 (see, world wide web, at the URL “biology.duke.edu/chlamy_genome/chloro.html”, and clicking on “view complete genome as text file”; see, also, “maps of the chloroplast genome” link, then “140-150 kb” link for Eco site at about 143.1 kb and Xho site at about 148.5 kb). The Eco/Xho chloroplast genome sequence was inserted into Eco RI/Xho I digested the pBS plasmid (Stratagene Corp., La Jolla Calif.). The Bam HI site in p322 corresponds to that beginning at position 146522 of the chloroplast genomic DNA sequence.

Southern and Northern Blots

Southern blots and ³²P labeling of DNA for use as probes were carried out as described in Sambrook et al., supra, 1989). Radioactive probes used on Southern blots included the 2.2 kb Bam HI/Pst I fragment of p322 (probe 5′ p322), the 2.0 kb Bam HI/Xho I fragment of p322 (probe 3′ p322) and the 717 bp Nde I/Xba I fragments from p53rGFPct (probe GFPct) or p53rGFPncb (probe GFPncb). These latter two probes were also used to detect GFPct and GFPncb mRNAs on Northern blots. Additional radioactive probes used in northern blot analysis included the psbA and rbcL cDNAs. Northern blots and Southern blots were visualized utilizing a Packard Cyclone Storage Phosphor System equipped with the OPTIQUANT software package.

Protein Expression, Western Blotting and Fluorescence Gels

Plasmids pETGFPct and pETGFPncb were transformed into E. coli strain BL21 and 6 His-tagged GFPct or GFPncb protein expression induced by IPTG according to the manufacturer's protocol (Novagen). Purification of His-tagged proteins was carried out using Ni-agarose affinity chromatography (Qiagen). Western blots were carried out as described in Cohen et al. (supra, 1998) using a mouse anti GFP primary antibody (Clontech) and an alkaline phosphatase labeled anti-mouse secondary antibody (Sigma). Fluorescence gels were run as for gels intended for Coomassie staining or western transfer, except that proteins were not boiled prior to loading. GFP was visualized in gels by viewing with a Berthold Night Owl CCD camera, model LB 981, equipped with 485 nm excitation and 535 nm emission filters (Chroma Corp.). Images were generated using WinLight software.

Generation of Excitation Spectra for GFPct and GFPncb

Excitation spectra were generated with affinity purified GFPct or GFPncb proteins on a Perkin Elmer Luminescence Spectrometer Model LS50. Recombinant proteins were diluted in 50 mM NaH₂PO₄, 300 mM NaCl, 250 mM imidazole, pH 8.0, prior to reading on the spectrometer. Excitation spectra were generated by scanning illumination from 350 to 550 nm, while monitoring emission at 510 nm.

De Novo Synthesis of a GFP Gene in C. reinhardtii Chloroplast Codon Bias

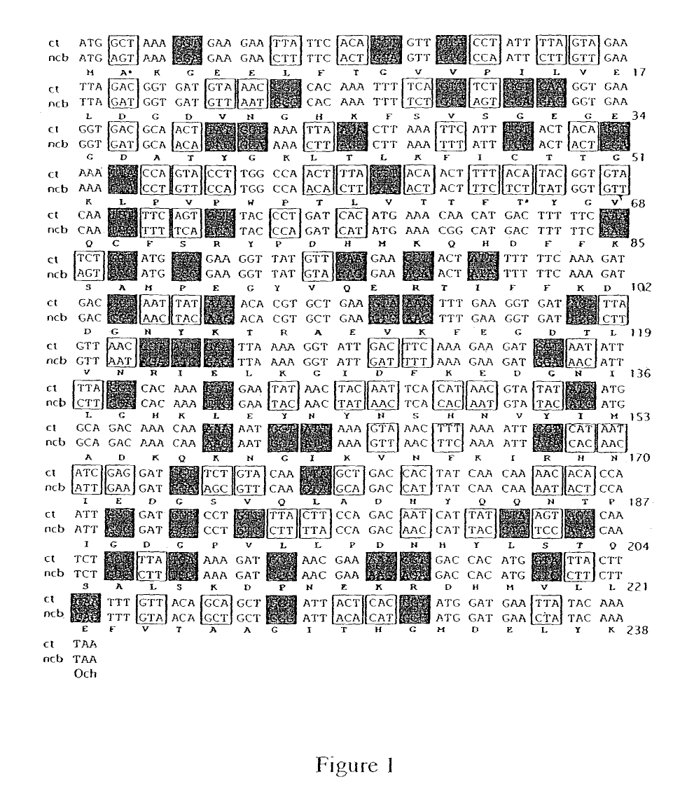

To develop a robust reporter gene for expression in the C. reinhardtii chloroplast, a green fluorescent protein gene, whose codon usage was optimized to reflect that of the C. reinhardtii chloroplast genome, was synthesized. Two amino acid changes to the native GFP (GFPncb) coding region were designed to enhance the fluorescent and expression properties of the protein. The first of these amino acid changes, which was not expected to impact the spectral qualities of GFP, was a serine to alanine change at amino acid position 2, to place the initiation codon in a more favorable context. The second change, a serine to threonine change at amino acid position 65 was made to enhance the amplitude of excitation at 485 nm relative to native GFP (approximately 6 fold), while at the same time reducing excitation at 395 nm (Heim et al., Nature 373:663-664, 1995, which is incorporated herein by reference). This change was introduced into the GFPct coding sequence to improve fluorescent detection using visible light. As shown in FIG. 1, there also was an amino acid change, Q80R, in the GFPncb gene that was not in the wt GFP gene. This alteration was introduced during PCR amplification of the native GFP gene, prior to selection of the clone. This Q80R mutation is a common alteration found upon amplification of native GFP coding sequences using PCR (Tsien, supra, 1998) and has no effect on protein function. As such, this change was included in the GFPct gene for consistency.

Characterization of E. coli Expressed GFPct and GFPncb

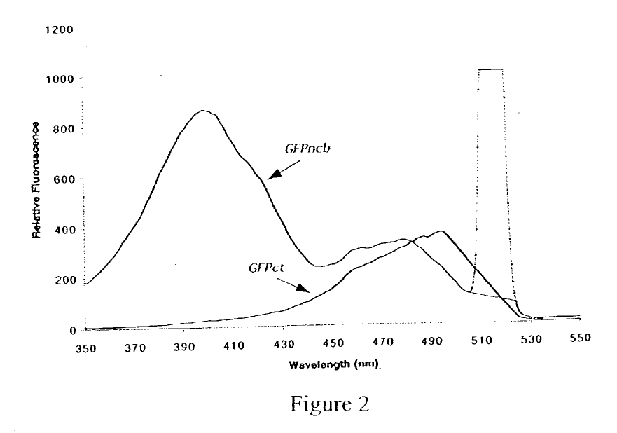

To determine if the GFPct and GFPncb genes were capable of producing functional GFP protein, E. coli cell lysates prepared from cells transformed with either pETGFPct or pETGFPncb were examined. Ni affinity chromatography of E. coli lysates produced proteins of the correct molecular mass for GFP. Direct fluorescence assays of SDS PAGE separated E. coli produced proteins revealed that both proteins fluoresced under blue light illumination, and showed slightly different fluorescent properties consistent with the introduced amino acid changes. The S65T alteration to the GFPct protein resulted in greatly enhanced level of fluorescence at 485 nm (only ⅕ the amount of E. coli expressed GFPct protein was used in this assay relative to GFPncb protein), while its fluorescence at 395 nm excitation is greatly reduced (see FIG. 2). Western blot analysis using a mouse polyclonal antibody raised against native GFP showed a similar signal for both GFPct and GFPncb. This result is particularly important given that the spectral qualities of the GFPct protein was intentionally enhanced relative to the GFPncb protein. Thus, while fluorescence detection, based upon excitation in the visible (485 nm), would favor GFPct detection, immunolabeling is nondiscriminatory, allowing for the direct comparison of GFPct and GFPncb protein accumulation in C. reinhardtii chloroplasts.

Southern and Northern Blot Analysis of GFPct and GFPncb Transformants

Upon demonstrating that the GFPct and GFPncb coding sequences were capable of producing functional GFP proteins, C. reinhardtii chloroplasts were transformed with pExGFPct and pExGFPncb. In addition, the cells were cotransformed with the selectable marker plasmid, p228, which confers resistance to spectinomycin. Primary transformants were screened by PCR followed by Southern blot analysis, and positive transformants were taken through additional rounds of selection to isolate homoplasmic lines, in which all copies of the chloroplast genome contained the introduced GFP gene.

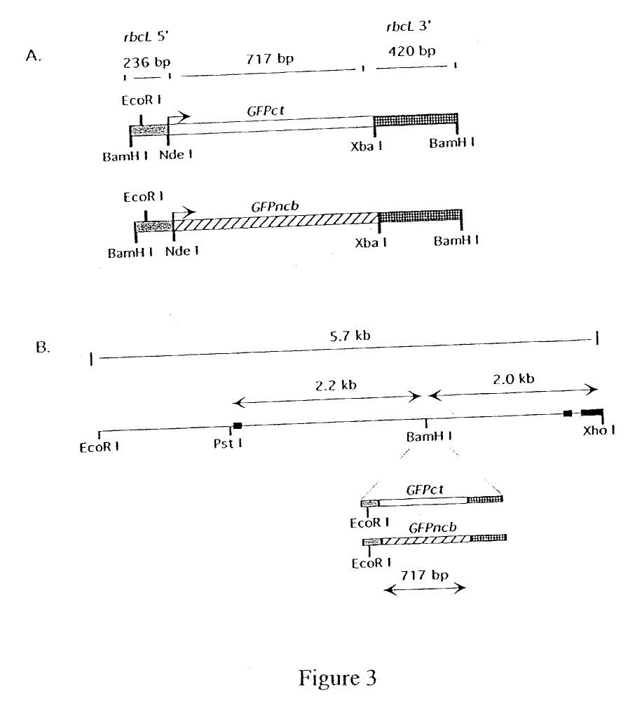

Two homoplasmic GFPct transformants, 18.3 and 21.2, and two homoplasmic GFPncb transformants, 5.8 and 12.1, were selected for further analysis (see FIG. 3A, showing GFPct and GFPncb constructs with relevant restriction sites indicated). Correct integration of the 7.1 kb Eco/Xho region of plasmids pExGFPct and pExGFPncb into the chloroplast genome was ascertained using the probes indicated on the map of the genes (FIG. 3B). Genomic DNA from wt and the GFPct and GFPncb transformants was digested with Eco RI and Xho I, fractionated on agarose gels, and subjected to Southern blot analysis. Because the rbcL 5′ UTR contains an Eco RI restriction site (FIG. 3A), digestion of transformant DNA with Eco RI/Xho I should result in a smaller fragment hybridizing to either the 5′ or 3′ p322 probes relative to wt DNA.

Southern blot analysis of GFPct and GFPncb C. reinhardtii chloroplast transformants demonstrated that the transgenic lines were homoplasmic. C. reinhardtii DNA was digested simultaneously with Eco RI and Xho I, and filters were hybridized with the radioactive probe. The 5′ p322 ³²P labeled probe and the 3′ p322 ³²p labeled probe, hybridized to Eco RI fragments of 3.7 kb and 3.3 kb, respectively, in the GFPct and GFPncb transformants. These same probes, however, hybridized to a 5.7 kb Eco RI/Xho I fragment in the non-transformed wt C. reinhardtii strain, as expected. The DNA blots were stripped and re-probed with GFPct and GFPncb specific probes. An Eco RI/Xho I fragment of 3.3 kb was detected in transformants 5.8 and 12.1 using the GFPncb probe (FIG. 4, central panel), and a similar sized fragment was identified in transformants 18.3 and 21.2 using the GFPct probe. No signal was detected in wt C. reinhardtii DNA using either GFP probe.

Accumulation of GFP mRNA in Transgenic Strains

Northern blot analysis of total RNA was used to determine if the GFPct and GFPncb genes were transcribed in transgenic C. reinhardtii chloroplasts. Ten μg of total RNA isolated from wt and transgenic lines 5.8, 12.1, 18.3 and 21.2 was separated on denaturing agarose gels and blotted to nylon membrane. Duplicate filters were hybridized with either a ³²P labeled psbA or rbcL cDNA probe. Each of the strains accumulated psbA and rbcL mRNAs to similar levels, demonstrating that equal amounts of RNA were loaded for each lane, and that chloroplast transcription and mRNA accumulation are normal in the transgenic strains.

The filters were stripped and re-probed with the GFPct and GFPncb specific probes. Strains 5.8 and 12.1 accumulated GFPncb mRNA, while strains 18.3 and 21.2 accumulated GFPct mRNA. No GFP signal was observed in wt cells, as expected. All four cDNA probes were labeled to approximately the same specific activity, and while the GFPct and GFPncb signals were similar, both GFP probed filters required longer exposures (approximately four times) to obtain a similar signal to the rbcL probe. These results indicate that the GFP mRNAs accumulate to roughly one quarter the level of the endogenous rbcL mRNA.

Analysis of GFP Accumulation in Transgenic C. reinhardtii Chloroplasts

To determine the levels of GFPct and GFPncb protein accumulation in the transgenic lines, GFP was measured by both fluorescence and western blot analysis. Comparison of GFP accumulation in C. reinhardtii transgenic strain 21.2 expressing GFPct, and strains 5.8 and 12.1, both expressing GFPncb. Cells were grown to a density of 1×10⁷ cells per ml under continuous light (5,000 lux), conditions known to allow maximal accumulation of GFP. Total soluble protein was subjected to SDS-PAGE, followed by western blot analysis with anti-GFP antisera. Twenty μg of total soluble protein was loaded for GFPncb transgenic strains 5.8 and 12.1, while 250 ng (1/80) to 20 μg (1/1) total soluble protein was loaded for GFPct transgenic 21.2.

Six μg of total soluble protein (tsp) was separated by SDS-PAGE and the resulting gels subjected to either Coomassie staining, fluorescence imaging, or western blot analysis. The Coomassie stained gel (6 μg total soluble protein, isolated from the indicated C. reinhardtii strains was subjected to 12% SDS-PAGE) indicated that equal amounts of protein were loaded in each lane. The fluorescence gel (proteins were prepared as for Coomassie stain gels, except samples were not boiled prior to loading; protein separated by SDS-PAGE)—excitation was set at 485 nm and emission was set at 535 nm. imaged at 485 nm excitation, 535 nm emission—shows a signal only for the GFPct transformants 18.3 and 21.2. No fluorescent signal was observed for any GFP transformant when excited at 366 nm. shows GFPct and GFPncb proteins expressed in chloroplast in transgenic C. reinhardtii strains.

Western blot analysis of the same samples showed similar results to the fluorescent analysis, with no GFP detected in the GFPncb transformants, and a good signal in the GFPct strains (western blot analysis of chloroplast expressed GFP proteins transferred to nitrocellulose and probed with anti-GFP antisera). Titration was performed to more precisely ascertain the difference in GFP accumulation between GFPct and GFPncb transformants. Twenty μg of tsp from GFPncb transformants 5.8 and 12.1 were separated along with tsp from GFPct transformant 21.2. For the GFPct strain, protein concentrations ranged from 20 μg to 250 ng. A comparison of samples indicated that the level of GFPct accumulation in the 21.2 transformant was approximately 80 fold higher than that seen in either of the GFPncb transformants.

Use of Chloroplast Optimized GFP as a Reporter of Chloroplast Gene Expression

The effect of different growth conditions on GFPct accumulation in transgenic lines was examined to confirm the ability of the GFPct gene to act as a reporter of chloroplast gene expression. C. reinhardtii GFPct transgenic strain 21.2 was maintained under constant illumination at a density of 1×10⁶ cells per ml at either 5,000 lux (high light) or 450 lux (low light), prior to harvesting. Western blot analysis was carried out on 1 μg tsp from each treatment. The effect of light intensity on accumulation of GFPct in C. reinhardtii was examined. Prior to harvest, C. reinhardtii transgenic line 21.2 was maintained at either 1×10⁶ cells per ml or 1×10⁷ cells per ml for at least 48 hr under constant illumination at the indicated light intensity. Total soluble protein (1 μg) was subjected to 12% SDS-PAGE and western blotting with anti-GFP primary antibody.

Cells maintained at 1×10⁶ cells per ml under constant illumination of 5,000 lux accumulated roughly 10% as much GFPct as cells maintained at 1×10⁶ cells per ml under low light flux. When a third flask was maintained at a density of 1×10⁷ cells per ml under 5,000 lux, constant illumination, GFP again accumulated to high levels, as the high cell density acted to reduce light intensity within the growing culture, in essence creating a low light environment. These results demonstrate that the GFPct gene can be used to report differences in protein synthesis based upon subtle changes in environmental conditions, and demonstrate the usefulness of the GFPct gene as a reporter of chloroplast gene expression.

Several heterologous genes have been employed as reporters of chloroplast gene expression in C. reinhardtii, but their utility has been limited due to low levels of protein expression. There are several possible explanations for the low levels of heterologous protein expression in C. reinhardtii chloroplasts. For example, the promoters used to drive transcription of these genes may result in low levels of transcription. Alternatively, some of these reporter mRNAs may be inherently unstable, resulting in low levels of mRNA accumulation. Another possibility is that RNA elements required for translation may be lacking from these chimeric mRNAs. Strong codon bias in C. reinhardtii chloroplast genes also may preclude the translation of heterologous mRNAs.

Although promoter activity and mRNA stability greatly impact gene expression in chloroplasts, analysis of transgenic C. reinhardtii chloroplasts has shown sufficient heterologous mRNA accumulation to support high levels of protein synthesis. Additionally, in most cases C. reinhardtii 5′UTRs and 3′UTRs were used in construction of the chimeric genes, making it unlikely that critical RNA elements were lacking from these reporter mRNAs. As disclosed herein, altered codon usage was used as a means to enhance heterologous protein accumulation in the C. reinhardtii chloroplast. The altered codon usage method was exemplified using the A. aequeorea green fluorescent protein (GFP).

The GFP coding region of GFP was engineered to match the codon usage of protein coding sequences from the C. reinhardtii chloroplast genome. Expression of this GFPct gene, as well as a native GFP gene (GFPncb), was placed under the control of the C. reinhardtii chloroplast rbcL 5′ and 3′ UTRs. Both the GFPncb gene and the GFPct gene were transcribed and accumulated mRNA to similar levels in transgenic C. reinhardtii chloroplasts.

Transgenic strains expressing GFPct accumulated approximately 80 fold more GFP than those expressing GF [symbol] ncb. The GFPct producing strain 21.2 accumulated GFP to approximately 0.5% of the total soluble protein, under optimal growth conditions. This level of protein expression allows for analysis of GFP expression by fluorescence assays of total cellular proteins. Previous reports of uidA (GUS) expression in C. reinhardtii chloroplast under the control of the rbcL 5′ and 3′ UTRs showed low levels of protein expression, approximately 0.01% of soluble protein; this level of GUS accumulation was similar to the level of GFP accumulation obtained with the GFPncb gene using the same rbcL control elements (Ishikura et al., supra, 1999, also reporting relatively low levels of rbcL-GUS mRNA accumulation) (similar to the low levels for rbcL GFP mRNA, as disclosed herein).

There were a total of 123 codon changes is the GFPct gene as compared to the GFPncb gene, including 121 synonymous codon changes, and two codons having amino acid substitutions (see above). Of the 121 synonymous codon changes, 66 changes represented only a modest shift toward a more optimized codon usage. Of the remaining codons, 54 were changes that resulted in an infrequently used codon being replaced with a frequently used codon. The codon optimization is fairly evenly distributed throughout the GFP gene, with 15 alterations in the first third of the coding region, 20 in the second third and 18 in the final third.

An analysis of genes previously expressed in C. reinhardtii chloroplasts, including Renilla luciferase (Minko et al., supra, 1999), uidA (Sakamoto et al., supra, 1993) aadA (Goldschmidt-Clermont, supra, 1991) and aph A6 coding sequences (Bateman and Purton, supra, 2000) revealed 61, 252, 121 and 65 non-preferred codons in each of these respective genes. If the number of non-preferred codons in these reporter genes is expressed as a percentage of their total codons, values of 20%, 42%, 46%, and 25%, respectively, are obtained. This compares with the GFPncb gene where non-preferred codons account for 23% of the total codons. These results demonstrate that expression of these other reporters in C. reinhardtii chloroplasts can be greatly enhanced by altering codon usage.

Since the base composition of the GFP sequence had been significantly changed, the effect of these changes on the structure of the mRNA was examined for the GFPct and GFPncb mRNAs. This analysis ensured that the enhancement of translation in the GFPct mRNA was due to the differences in codon usage, rather than to some effects of mRNA secondary structure that could preclude loading of the GFPncb onto ribosomes. The first 250 nucleotides of the GFPct and GFPncb mRNAs were examined using the RNA folding program mfold (Zucker et al., In RNA Biochemistry and Biotechnology 11-43 (ed. Barciszewski and Clark, NATO ASI Series, Kluwer Acad. Publ. 1999; Matthews et al., J. Mol. Biol. 288:911-940, 1999). No significant secondary structure differences were predicted between the two genes, with the free energy of the most favorable structures being −42 kcal for GFPct and a similar −38 kcal for the GFPncb sequence.

The results disclosed herein demonstrate that optimizing codon usage can facilitate translation and expression of a polypeptide, as exemplified by the optimized GFPct gene, which was used as a reporter of chloroplast gene expression C. reinhardtii. The demonstration that codon optimization can be used to achieve high levels of recombinant protein expression in C. reinhardtii indicates that codon optimization generally can contribute to translation efficiency of other heterologous polypeptides in plant chloroplasts. The relatively low levels of GFP mRNA accumulation as compared to the endogenous rbcL mRNA indicates that optimizing promoter activity and mRNA stability of GFPct can provide a means to enhance the signal of GFPct to even higher or more desirable levels. As such, the GFPct gene provides a tool that is useful to conveniently optimize transcription, mRNA stability and translation of GFP in plant chloroplasts, including in C. reinhardtii chloroplasts.

### EXAMPLE 2

### Characterization of Plant Chloroplast Ribosome Binding Sequence (RBS)

This example demonstrates the identification and characterization of ribosome binding sequences that direct translation in chloroplasts.

Mutant Construction and Characterization

Site-specific mutations were generated by PCR amplification of the psbA 5′UTR using the following oligonucleotides:

| | |
|---|---|
| 5′-GAAGCTTGAATTTATAAATTAAAATATTTTTACAATATTTTACCCAGA | (RBS-Alt; SEQ ID NO:23) |
| AATTAAAAC-3′; | |
| 5′-TGTCATATGTTAATTTTTTTAAAGTTTTTCTCCGTAAAATATTG-3′; | (RBS-23; SEQ ID NO:24) |
| 5′-TGTCATATGTTAATTTTTTTAAAGTCTCCGTAAAATATTG-3′; | (RBS-19; SEQ ID NO:25) |
| 5′-TGTCATATGTTAATTTTTTTTCTCCGTAAAATATTG-3′; | (RBS-15; SEQ ID NO:26) |
| 5′-GTCATATGTTAATTTCTCCG-3′; and | (RBS-11; SEQ ID NO:27) |
| 5′-TGTCATATGTTAATCCTCCTAAAGTTTTAATTTCTCCG-3′. | (RBS-Add; SEQ ID NO:28) |

Plasmid construction and C. reinhardtii transformation were performed as described by Mayfield et al. (supra, 1994). The 16S-1470/71 and 16S-1467/68 mutants were constructed using a QUICK-CHANGE mutagenesis kit (Qiagen). Mutants were characterized by northern blot and western blot analysis. RNA isolation, northern blot analysis, protein isolation, western blot analysis, and in vivo pulse-labeling of proteins with (¹⁴C)-acetate were performed as described by Cohen et al. (supra, 1998).

For “toeprinting” analysis, 30S ribosomal subunits were isolated as described by Harris (Microbiol. Rev. 58:700-754, 1989, which is incorporated herein by reference), with minor modifications. Wild type C. reinhardtii cells (2137a) were resuspended in TKMD buffer (25 mM Tris-HCl (pH 7.8), 25 mM KCl, 25 mM MgOAc, 5 mM DTT) and broken with one passage in a French press at 5000 psi. The cell exudate was centrifuged at 40,000×g at 4° C. for 30 min in a Beckman JA-20 rotor. 200 A₂₆₀ units of the supernatant was placed over a 10-30% linear sucrose gradient in TKMD buffer containing 100 mM KCl for one step preparation of 30S and 50S ribosomal subunits. The gradients were centrifuged for 20 hr at 2° C. at 22,500 rpm in a Beckman SW28.1 rotor. The gradients were processed using an optical scanner and fraction collector reading the Absorbance at 260 nm. 30S and 50S fractions were pooled and diluted 1:1 with high salt TKMD containing 800 mM KCl and centrifuged at 200,000×g for 20 hr at 4° C. Beckman TLA-100 rotor. The pellets were resuspended in TKMD buffer containing 100 mM KCl and frozen in liquid nitrogen for storage at −70° C. The degree of cross contamination of the 30S and 50S subunits was assayed using RNA blot analysis (Cohen et al., supra, 1998).

Formation of the initiation complex was assayed by extension inhibition as described by Hartz et al., (J. Mol. Biol. 218:83-97, 1988, which is incorporated herein by reference), with minor modifications. Annealing mixtures contained 0.6 pmol of the 5′-(³²P)-end labeled oligonucleotide and 0.2 pmol of the synthetic psbA D1-HA transcript in a 10 μl reaction mixture (see Example 2). Extension inhibition was initiated by the addition of 3.75 mM dNTPs plus 8×10⁻⁵ to 2×10⁻³ μM high salt washed 30S ribosomal subunits. After incubation of the reaction at 37° C. for 5 min, uncharged E. coli tRNA (tRNA_{f}^{met}; Roche Diagnostics) was added to a final concentration of 5 μM. AMV reverse transcriptase (0.5 units) was added, and the reaction was incubated at 37° C. for an additional 15 min. The reactions were analyzed on an 8% sequencing gel. Sequencing reactions were performed as described above using dNTPs at a final concentration of 200 μM in the absence of ribosomes or tRNA.

Gel Shift Assays

Approximately 1 μg heparin-agarose purified protein (Cohen et al., 1998) was incubated with 0.4 units PRIME RNase Inhibitor (5 Prime→3 Prime, Inc.) for 10 min at room temperature in a total volume of 8 μl dialysis buffer (20 mM Tris-HCl (pH 7.5), 100 mM KOAc, 0.2 mM EDTA (pH 8.0), 2 mM DTT, 20% glycerol, 4 mM MgCl₂). The reaction was incubated at room temperature for 10 min upon addition of 0.04 pmol of in vitro transcribed (³²P)-labeled psbA RNA, spanning the positions −90 to +171 relative to the translation start codon, 20 μg of wheat germ tRNA (Sigma), and 3 μg of FuD7 (a C. reinhardtii strain lacking psbA mRNA) total RNA. In some reactions, 10 pmol unlabeled in vitro transcribed unlabeled psbA RNA was added as a competitor. RNA/protein complexes were separated in a 5% non-denaturing polyacrylamide gel.

Chloroplast 30S Ribosomal Subunits Recognize a Shine-Delgarno Ribosome Binding Sequence in the psbA 5′UTR

To identify RNA elements required for chloroplast mRNA translation, variant psbA genes containing site-specific mutations within the 5′UTR were introduced into chloroplasts of a psbA-deficient strain of C. reinhardtii (Mayfield et al., supra, 1994). A potential RBS within the psbA 5′UTR located 27 nucleotides upstream of the start codon was identified based on its potential to recognize the anti-SD sequence within the chloroplast 16S rRNA. Deletion of this sequence (RBS-del) resulted in a failure of the psbA mRNA to associate with ribosomes, and in the complete loss of synthesis of the corresponding D1 protein (Mayfield et al., supra, 1994). While this result suggested that the element may function as an RBS, the deletion also may have affected ribosome binding by a number of alternative mechanisms, including direct or indirect disruption of binding sites for trans-acting factors that bind the 5′UTR adjacent to the RBS (Yohn et al., Proc. Natl. Acad. Sci., USA 95:2238-2243, 1998a; Yohn et al., J. Cell Biol. 142:435-442, 1998b; Danon and Mayfield, EMBO J. 10:3993-4001, 1991, each of which is incorporated herein by reference; see, also, Fargo et al., supra, 1998).

SD sequences within RBS elements promote the initiation of translation from prokaryotic transcripts by pairing to a complementary sequence (anti-SD sequence) at the 3′ end of the 16S rRNA of the 30S small ribosomal subunit (Voorma, In Translational Control (ed. Hershey et al., Cold Spring Harbor Laboratory Press 1996), which is incorporated herein by reference). This interaction has been measured in vitro using purified 30S ribosomal subunits added to prokaryotic transcripts (Hartz et al., supra, 1991). Bound 30S subunits block extension of a downstream oligonucleotide primer on the mRNA resulting in a ribosomal “toeprint”.

In order to determine if 30S subunits would recognize the RBS within the 5′UTR of the psbA mRNA, 30S ribosomal subunits were isolated from C. reinhardtii. The 30S subunits were free of contaminating 50S ribosomal subunits. A 5′-(³²P)-end labeled oligonucleotide primer complementary to a region of the psbA mRNA downstream of the initiation codon was annealed to purified in vitro synthesized psbA transcripts. The ³²P-oligonucleotide/RNA complexes were incubated with increasing concentrations of purified C. reinhardtii 30S ribosome subunits, and E. coli fMet tRNA (see Example 2). Pause sites during primer extension occurred due to bound ribosomal subunits. Sequencing reactions were performed in parallel to determine the position of the bound ribosome. In reactions containing 30S ribosomes, a pause in the toeprint reaction occurred 12 nucleotide 3′ of the Shine-Delgarno sequence (RBS pause) and 12 nucleotides 3′ of the initiation codon (AUG pause). Primer extension toeprints were observed when chloroplast 30S ribosomal subunits were incubated with an RNA transcript corresponding to the 5′ end of the psbA mRNA. These pauses occur approximately 12 nucleotides downstream of both the putative SD sequence and the start codon, consistent with 30S ribosomal subunits bound at both of these two sequences. Binding of E. coli 30S subunits onto the psbA mRNA from barley also revealed a toeprint corresponding to a potential SD sequence positioned in a similar location to that of the psbA mRNA from C. reinhardtii (Kim and Mullet, Plant Mol. Biol. 25:437-448, 1994, which is incorporated herein by reference). These results indicate that the putative RBS elements have characteristics of functional RBS elements. Thus, the in vitro biochemical data supports the interpretation of the in vivo genetic evidence from the previous study, that an RBS element in the psbA mRNA is 27 bases 5′ (upstream) of the start codon (Mayfield et al., supra, 1994).

Mutation of the Anti-SD Sequence in the 16S rRNA Inhibits Translation from a Subset of Chloroplast mRNAs

In order to demonstrate that chloroplast ribosomes recognize messages via interaction with the SD sequence, two homoplasmic C. reinhardtii strains were constructed, in which the anti-SD sequence within the chloroplast 16S rRNA was mutated. Nucleotides within the anti-SD sequence located at the 3′ end of the 16S rRNA were changed from CCUCC to GGUCC (nucleotides 1467 and 1468 of the 16S rRNA) or from CCUCC to CCUGG (nucleotides 1470 and 1471 of the 16S rRNA; see, also, SEQ ID NO:29). These mutants were viable when cultured in the presence of complete media capable of supporting growth in the absence of photosynthesis, and did not exhibit any gross morphological defects arising from alterations in chloroplast biogenesis. The 16S-1467/68 mutant strain was able to grow at a reduced rate on minimal media, whereas the 16S-1470/71 mutant strain was unable to grow on minimal media, indicating a reduction and elimination, respectively, of photosynthetic function in these mutants.

Accumulation of chloroplast-encoded proteins in these strains was examined by western blot analysis. Equal quantities of total protein (determined by Coomassie Blue staining) prepared from the wild type (wt) or mutant C. reinhardtii strains 16S-1467/68 and 16S-1470/71 were separated by SDS-PAGE, blotted to nitrocellulose, and treated with rabbit polyclonal antisera specific for the D1, D2, ATPase, or Lsu proteins. Mutation of the anti-SD sequence in the 16S rRNA affected the accumulation of some chloroplast proteins. The psbA-encoded. D1 protein failed to accumulate in the 16S-1470/71 mutant, and accumulated to only 20% of wild type levels in the 16S-1467/68 mutant. The psbD-encoded D2 protein showed a similar pattern, accumulating to less than 10% of wild type in the 16S-1470/71 mutant and to about 25% in the 16S-1467/68 mutant Accumulation of the chloroplast ATPase was also impaired in the 16S-1470/71 mutant (50% of wild type levels), although present at near wild type levels in the 16S-1467/68 mutant. Conversely, accumulation of the soluble chloroplast-encoded large subunit of Rubisco (Lsu) was largely unaffected in either 16S mutant strain.

Failure of the D1 protein to accumulate in the mutant strains indicated that Shine-Delgarno interactions between the putative RBS element and the 16S rRNA are required for optimal translation. The failure of the D2 protein to accumulate in these strains can be a result of the psbD mRNA requiring the same anti-SD sequence as the psbA mRNA for translation, or due to loss of the D1 subunit resulting in a destabilization of the D2 protein after synthesis. For example, nuclear mutants of C. reinhardtii that fail to synthesize individual PSII subunits fail to accumulate other core chloroplast-encoded PSII polypeptides, although these proteins are synthesized at wild type rates (Erickson et al., EMBO J 5:1745-1754, 1986).

In order to examine the rate of translation of individual chloroplast proteins, the wild type strain, strains carrying the mutated 16S rRNA, and a C. reinhardtii strain lacking the psbA gene, were pulse-labeled with (¹⁴C)-acetate. The 16S-1470/71 mutation resulted in the absence of protein synthesis of almost all of the membrane proteins including the D1, D2, P5, and P6 proteins. Equal amounts of total membrane-associated or soluble protein (Cohen et al., Meth. Enzymol. 297:192-208, 1998, which is incorporated herein by reference; see, also, Example 2), as determined by Coomassie Blue staining, prepared from the wild type and mutant C. reinhardtii strains pulse-labeled with (¹⁴C)-acetate were resolved by SDS-PAGE. (¹⁴C)-labeled proteins were visualized by autoradiography. Mutations of the 16S rRNA anti-SD sequence reduced the rate of protein synthesis of several chloroplast-encoded proteins. This result indicates that the reduction in D2 accumulation was not due to a lack of D1 accumulation, and that an anti-SD sequence was required for psbD translation. Translation of the ATPase mRNAs was also reduced in this strain, although to a lesser degree than the other membrane proteins. A less severe affect was observed for the 16S-1467/68 mutant, consistent with the observed levels of protein accumulation. Some membrane-associated proteins continued to be translated in the 16S-1470/71 strain at wild type levels. In stark contrast to the membrane-associated proteins, almost no change in the rate of soluble chloroplast protein translation was observed in the 16S rRNA mutants. Synthesis of soluble proteins at wild type rates demonstrates that the chloroplast ribosomes bearing alterations in the anti-SD element of the 16S rRNA are functional and capable of supporting translation. These results indicate that the regulation of expression of soluble and membrane proteins in the chloroplast can be differentially regulated via an RBS-dependent mechanism.

Expression of the psbA-Encoded D1 Protein Requires the Presence of a SD Sequence in the RBS with Unique Spacing Requirements

The role of the RBS in psbA mRNA translation was further investigated using C. reinhardtii strains, in which the RBS was changed from GGAG to CCAG (RBS-Alt). Each strain was grown under continuous illumination in complete (TAP) media (see Example 2) and equal quantities of membrane proteins (determined by Coomassie Blue staining) were separated by SDS-PAGE, blotted to nitrocellulose, and treated with rabbit polyclonal antisera specific for the D1 protein. Multiple bands arose from bound chlorophyll as a result of incomplete denaturation of the D1 protein. The RBS-Alt mutation eliminates the SD base-pairing potential between the psbA mRNA and the 3′ terminus of the 16S rRNA, without disrupting the relative location of other elements within the 5′UTR (see FIG. 4). As previously shown for the RBS-del (Mayfield et al., supra, 1994), the D1 protein failed to accumulate in RBS-Alt. This result demonstrates that the GGAG sequence is required for psbA expression as expected for an authentic RBS.

If, as believed, the 30S ribosomal subunit is unable to simultaneously contact both the RBS and the initiation codon if these sequences are greater than 15 nucleotides apart (Chen et al., Nucl. Acids Res. 22:4953-4957, 1994), the putative SD sequence in the psbA mRNA, which is located 27 nucleotides from the psbA initiation codon, should be unable to direct translation initiation at the proper start codon. To examine how the relative location of the RBS of the psbA mRNA influences expression, a series of deletions were introduced into the 5′UTR to position the RBS element closer to the initiation codon (FIG. 4). As the RBS was moved progressively closer to the initiation codon, D1 protein accumulation decreased in C. reinhardtii cells. Deletions that positioned the RBS near the optimal location for prokaryotic RBS elements (RBS-15, RBS-11), resulted in no D1 protein accumulation in C. reinhardtii chloroplasts. Furthermore, the addition of a traditional prokaryotic RBS element seven nucleotides upstream of the initiation codon (SD-Add) failed to enhance D1 accumulation in the presence of the wild type psbA RBS sequence. Failure to accumulate the Dl protein in the psbA mutant strains is not due to a loss of mRNA stability

While loss of D1 accumulation in the strains bearing mutations to the putative SD sequence in the psbA 5′UTR can be explained by the loss of ribosome recognition, alternative explanations exist. For example, mutations that destabilize transcripts often result in reduced mRNA accumulation levels, which can lead to reduced translation/protein accumulation. psbA mRNA accumulated in C. reinhardtii strains containing site-directed mutations affecting the RBS sequence. psbA mRNA levels from total or ribosome-associated RNA pools were visualized with a radiolabeled probe specific for psbA or 16S rRNA (to ensure equal loading). Relative psbA mRNA levels were corrected for differences in 16S rRNA, then normalized with respect to wild type.

Although mutations to the SD sequence in the psbA 5′UTR lead to a reduction in steady-state levels of accumulated psbA mRNA, the relative levels of accumulated mRNA did not correlate with the observed levels of D1 accumulation. For example, the D1 protein accumulated to greater levels in the RBS-23 mutant, despite a 50% reduction in psbA mRNA. The RBS-15 and RBS-11 strains were unable to accumulate any D1 protein, or grow under minimal growth conditions, but, nevertheless, accumulated the same amount of psbA mRNA as the RBS-19 mutant, which accumulated D1 protein. In fact, accumulation of just 10% of the wild type pshA mRNA level, as observed for the RBS-del and RBS-Alt mutants, was sufficient to observe wild type levels of D1 protein in other psbA mutants (Mayfield et al., supra, 1994). As such, the affects observed due to these mutations cannot be attributed to changes in mRNA stability/accumulation.

Loss of D1 protein accumulation also can occur if the mutation/deletion of the psbA 5′UTR might results in structural alterations that render the resulting transcripts untranslatable. To determine whether ribosomes can recognize the SD sequence, despite the presence of mutations that change the relative location of the SD sequence to the initiation codon, ribosome-associated RNA from each of the mutants was separated from free mRNA by centrifugation of cell extracts over a sucrose cushion. The strains containing the altered or deleted RBS had greatly reduced levels of psbA mRNA associated with ribosomes. However, each of the strains that contained an RBS element had significant (>50% wild type levels) psbA mRNA association with ribosomes, even in strains that fail to accumulate the D1 protein. Failure to accumulate D1 protein would indicate that the ribosome-associated RNA in the RBS-15 and RBS-11 mutants primarily consisted of RNA bound to monoribosomes rather than polyribosomes.

To further demonstrate that mutations that position the SD sequence closer to the start codon do not unintentionally prevent translation on 70S ribosomes, chimeric genes were constructed that contained the bacterial luciferase coding region placed behind the wild type or mutant psbA 5′UTR. The chimeric genes were transformed into E. coli and translation of the luciferase mRNA was measured by luminescence activity. The luciferase expression pattern in E. coli was inverse to that observed for D1 expression in C. reinhardtii. Mutations that position the psbA SD sequence closer to the initiation codon were newly competent for translation in bacteria. The coding regions of the bacterial luciferase genes (lux AB) from Vibrio harveyi were fused to either wild type (wt) or mutant psbA 5′UTR's and ligated into plasmids containing the wild type psbA promoter and 3′UTR. The plasmids were transformed into E. coli strain BL21 (DE3) and translation of luciferase was monitored by photon counting using a video camera (Welsh and Kay, Curr. Opin. Biotech. 5:617-622, 1997, which is incorporated herein by reference) in the presence of the luciferase substrate n-decyl aldehyde. The percentage of optimal expression (RBS-11) was determined for each strain. Luciferase was efficiently translated in bacteria from the constructs containing an RBS positioned 11 to 15 nucleotides upstream of the initiation codon, but was poorly translated when the RBS was positioned greater than 19 nucleotides upstream. This result contrasts with that reported for the 5′UTR of the atpB mRNA from C. reinhardtii, which was reported to drive translation in either bacteria or chloroplast at similar levels (Fargo et al., supra, 1998).

Sequences within the 5′UTR's of the psbA and psbD transcripts in C. reinhardtii can affect mRNA processing. The psbA 5′UTR is cleaved in vivo four nucleotides upstream of the RBS sequence and this maturation process correlates with ribosome association and is dependent on the presence of the RBS sequence (Bruick and Mayfield, supra, 1998). Analysis of the psbA 5′ terminus provides additional evidence that the psbA RBS sequences from the mutants are recognized by factors involved in the early stages of ribosome association. Primer extension analysis of the chloroplast psbA mutants demonstrated that the psbA 5′UTR was processed in each strain containing an RBS sequence, but not in the RBS-Alt and RBS-del mutants (see FIG. 4; see, also, Bruick, Graduate Thesis, The Scripps Research Institute, 1998). These results indicate that the RBS element in the RBS-11 and RBS-15 strains was recognized by ribosomal subunits in the chloroplast, but that this recognition, by itself, was not sufficient to direct proper translation initiation at the start codon.

Deletions to the psbA 5′UTR Do Not Prevent Association of Nuclear-Encoded, Trans-Acting Translation Factors

A nuclear-encoded protein complex specifically recognizes the psbA 5′UTR and dramatically enhances D1 protein synthesis by stimulating translation initiation (Danon and Mayfield, supra, 1991; Yohn et al., supra, 1998a; Yohn et al., supra, 1998b). To determine whether any of the psbA 5′UTR mutants affected the ability of this complex to bind to the mRNA, RNA binding affinity was measured for each of the mutant RNAs using an in vitro gel shift analysis. Gel shift analysis of binding of the psbA-specific complex to the psbA 5′UTR was performed. Radiolabeled RNA fragments corresponding to the wild type psbA 5′-terminus were transcribed in vitro and incubated in the presence of heparin-agarose purified proteins. RNA/protein interactions resulted in the retardation of the RNA on nondenaturing PAGE. A 250-fold excess of unlabeled competitor RNA also was added to some reactions. Excess unlabeled RNA corresponding to the psbA 5′UTR from each mutant was used to compete the binding of the protein complex to labeled RNA corresponding to the wild type psbA 5′ UTR. Each of the mutant psbA 5′UTRs was recognized by the protein complex in vitro, and only the RBS-11 RNA failed to fully compete the wild type RNA for binding of the protein complex. This result indicates that loss of translation in the majority of these mutants is not due to the elimination of a specific binding site for these translational activator proteins.

Having originated from an endosymbiotic prokaryote, the transcriptional and translational machinery of the chloroplast generally resembles that of bacteria. Chloroplast promoters contain elements similar to those of bacteria, and plastid promoters are capable of driving transcription in E. coli. The ribosomes of the chloroplast are clearly related to those of bacteria, and chloroplast ribosomal RNAs and ribosomal proteins show a high degree of conservation with their bacterial counterparts (Harris et al., supra, 1994). Chloroplast mRNAs also resemble prokaryotic mRNAs in that they are uncapped, generally not poly-adenylated, and can contain polycistronic messages. While the translational machinery in the chloroplast has retained its prokaryotic features, over time many regulatory responsibilities have been surrendered to the nucleus. How the prokaryote-like components of the chloroplast are integrated with the trans-acting regulatory factors introduced from the nucleus has remained largely unknown.

Due to the prokaryotic nature of the chloroplast translational machinery, Shine-Delgarno (SD) interactions were recognized early on as potential regulators of chloroplast translation. However, in most instances identifiable SD sequences were positioned too far from the start codon to be considered consensus RBS elements. Combined with mutagenesis studies in which bacterial-like consensus SD sequences were mutated without a loss of translation, the importance of SD interactions in chloroplast translation was dismissed (Fargo et al., 1998; Koo and Spremulli, 1994; Rochaix, 1996; Sakamoto et al., 1994).

In order to examine the impact of SD interactions on chloroplast translation in general, and on the translation of the psbA mRNA in particular, the anti-SD sequence within the chloroplast 16S rRNA was mutated to eliminate the base-pairing potential with putative SD sequences. The resulting ribosomes retained the ability to synthesize soluble chloroplast proteins, indicating that these 16S mutations did not generally suppress ribosome activity or function. However, the synthesis of most, but not all, membrane-associated chloroplast proteins was strongly impaired by the mutations to the anti-SD region of the 16S rRNA. These results establish the importance of the anti-SD region in chloroplast translation, and indicate that this element can be a component of translational regulation in plastids.

To examine the SD interaction from the mRNA side, a series of mutations were introduced to a potential SD sequence located 27 nucleotides upstream of the start codon in the psbA mRNA, which previously was implicated as an important element in psbA mRNA processing and translation (Bruick and Mayfield, supra, 1999; Mayfield et al., supra, 1994). As disclosed herein, mutations to the psbA SD element abolished mRNA/ribosome association and abolished psbA translation and D1 protein accumulation. Taken together with the 16S mutational analysis and the toeprinting assays, these results demonstrate that Shine-Dalgarno interactions are required for translation of the psbA mRNA, and for a subset of other chloroplast mRNAs.

In view of the unusual spacing between the SD element and the initiation codon in the psbA mRNA, the positional effects on SD function within chloroplasts was examined. A series of deletions that positioned the psbA SD element closer to that of the bacterial consensus resulted in a corresponding decrease in D1 translation in chloroplast, but rendered the transcripts competent for translation in bacteria. This result indicates that chloroplast and bacteria use a fundamentally different mechanism for identifying the initiation codon following a SD interaction. These results also demonstrate that the SD element within the psbA mRNA does not reside within the prokaryotic consensus for SD elements, and may explain why deletions of potential SD elements located at the bacterial consensus position in other plastid mRNAs had no effect on translation.

Because message stability, ribosome association, and translation are often intimately linked, it can be difficult to identify the primary effect of a mutation in the 5′UTR of a mRNA. It has been suggested that an RBS-like sequence (the AUGAG sequence: PRB2) positioned approximately 30 nucleotides upstream of the start codon in the psbD 5′ UTR affects D2 protein synthesis in the chloroplast by serving as a message stability element (Nickelsen et al., Plant Cell 11:957-970, 1999). Based on the loss of psbD translation in the 16S mutations and the position of the PRB2 element relative to the SD element of psbA, PRB2 likely is a SD element for the psbD mRNA. The reduction in psbD mRNA stability in mutants lacking this element, like those observed for the various mutations affecting the psbA SD, likely reflects the loss of ribosome association that would otherwise protect the mRNA from degradation (Wagner et al., J. Bacteriol. 176:1683-1688, 1994).

The contrast between translation of membrane proteins and soluble proteins in the 16S mutants indicates that the SD interaction can be a differential component of translational regulation in the chloroplast. Examination of membrane protein synthesis revealed that at least two membrane-associated proteins were translated at wild type levels in the 16S mutants. The differential translation of membrane proteins between the two 16S mutants indicates that chloroplast mRNAs can use slightly different sequences as SD elements, and suggests that two populations of ribosomes may exist in the chloroplast.

The location of the RBS element within the psbA mRNA is indicative of a novel mechanism in chloroplasts to promote migration of the early initiation complex from the RBS to the start codon. Secondary structures can shorten the distance between a typically positioned RBS elements in some prokaryotic messages. However, the nucleotides between the psbA RBS and the initiation codon can be substantially altered without loss of psbA translation, and this region is predicted to be relatively unstructured. A scanning mechanism, observed during translation initiation in eukaryotes, also was proposed for chloroplast mRNA, but requires ATP as an energy source for helicase activity, a characteristic not yet ascribed to chloroplast translation. Alternatively, chloroplast mRNAs may use protein factors to bring the 30S subunit, bound at the RBS sequence, into register with the initiation codon. One specific protein factor that binds to the 5′UTR of the psbA mRNA has homology with a eukaryotic protein known to interact with translation initiation factors (Yohn et al., supra, 1998a). Such eukaryotic-like proteins can bring the translation initiation complex to the correct initiation codon, thus functioning as translational regulators in the chloroplast. The additional spacing required between the RBS and the initiation codon can accommodate these protein factors, as most of the mutations examined herein did not prevent binding of the these factors in vitro. Analogous distal SD sequences also have been identified in the psbA 5′UTR of higher plants, indicating that such SD elements are characteristic for plant chloroplast mRNA.

### EXAMPLE 3

### Expression of Antibodies in Chloroplasts

This example demonstrates that chloroplast codon optimized polynucleotides encoding single chain antibodies are expressed in chloroplasts, and that the single chain antibodies assemble into dimers.

A polynucleotide (SEQ ID NO:15), which encodes a single chain antibody (HSV8; SEQ ID NO:16) that specifically binds herpes simplex virus (HSV) type 1 and HSV type 2, was transformed into C. reinhardtii chloroplasts using a pExGFP plant chloroplast vector (see Example 1), except that the polynucleotide encoding HSV8 (SEQ ID NO: 15) was substituted for the GFP coding sequence. Samples of total soluble protein from two transformants (10.6 and 11.3) were collected in the absence or presence of the reducing agent, dithiothreitol (DTT), separated by 10% SDS-PAGE using the Laemmli buffer system, and transferred to nitrocellulose filters (Cohen et al., supra, 1998) for western blot analysis. The HSV8 antibody, which contains an operatively linked FLAG peptide tag, was visualized using an anti-FLAG peptide tag antibody (M2 monoclonal antibody; Sigma) and an anti-mouse alkaline phosphatase conjugated antibody (Sigma).

HSV8 single chain antibody expressed in the two different transformants migrated at the expected apparent molecular mass (about 65 kDa). Remarkably, HSV8 antibodies isolated in the absence of DTT migrated as a dimer. These results demonstrate that protein complexes such as antibody dimers can assemble in plant chloroplasts. Similarly, a chloroplast codon biased synthetic polynucleotide (SEQ ID NO:42) encoding a single chain Fv fragment (SEQ ID NO:43) of the anti-HSV antibody was constructed and expressed in C. reinhardtii, and a functional single chain anti-HSV Fv antibody was obtained.

While combinatorial antibody libraries have solved the problem of access to large immunological repertoires, efficient production of these complex molecules remains a problem. Here, the efficient expression of a unique large single chain (lsc) antibody is demonstrated in the chloroplast of the unicellular, green alga, Chlamydomonas reinhardtii. High levels of protein accumulation were achieved by synthesizing the lsc gene in chloroplast codon bias and by driving expression of the chimeric gene using either of two C. reinhardtii chloroplast promoters and 5′ and 3′ RNA elements. This lsc antibody, directed against glycoprotein D of the herpes simplex virus, is produced in a soluble form by the alga and assembles into higher order complexes, in vivo. Aside from dimerization by disulfide bond formation, the antibody undergoes no detectable post-translational modification. Further, the results demonstrate that accumulation of the antibody can be modulated by the specific growth regime used to culture the alga, and by the choice of 5′ and 3′ elements used to drive expression of the antibody gene. These results demonstrate the utility of alga as an expression platform for recombinant proteins, and describe a new type of single chain antibody containing the entire heavy chain protein, including the Fc domain.

Currently, there are a number of heterologous protein expression systems available for the production of recombinant proteins, and each of these systems offers distinct advantages in terms of protein yield and ease of manipulation and cost of operation (1). Monoclonal antibodies (mAbs) are produced primarily by culture of transgenic mammalian cells in fermentation facilities. Due to high capital costs, and the inherent complexity of mammalian production systems, monoclonal antibody production capacity will fall substantially short of requirements over the next five years (2).

As a consequence of the projected shortfall in mAb production via mammalian cell culture, alternative, cost-effective, means to produce mAbs will be required to maintain the present pace of therapeutic protein development. Yeast and bacterial systems, while more economical in terms of media components, have several shortcomings including an inability to efficiently produce properly folded functional molecules, as well as poor yields of more complex proteins. In addition to traditional fermentation, several groups have sought to exploit the productivity of terrestrial plants for mAb production (3, 4, 5). In such systems, the plant itself becomes the bioreactor, with the antibody deposited into leaf or seed tissue. While plants afford an economy of scale unprecedented in the biotechnology industry (one can plant 1000s of acres in corn, for example), there are several inherent drawbacks to this approach as well. First, the length of time required from the initial transformation event to having usable (mg to gram) quantities of recombinant protein on hand, can be as long as three years for species such as corn. A second concern surrounding the expression of human therapeutics in food crops, is the potential for gene flow (via pollen) to surrounding crops (6), as occurred between transgenic corn expressing Bacillus thuringiensis insecticidal proteins and native landraces (7). These concerns raise the possibility that regulatory agencies will prohibit the open cultivation of transgenic food plants (like corn, rice and soybean) expressing human therapeutics.

Only a few attempts have been made to engineer chloroplasts for the expression of therapeutic proteins (8), although in some instances quite high levels of recombinant protein expression have been achieved in this organelle (9-12). There have been even fewer reports on the generation of transgenic algae for the expression of recombinant proteins, even though green algae have served as a model organism for understanding everything from the mechanisms of light and nutrient regulated gene expression to the assembly and function of the photosynthetic apparatus (13). As disclosed in Example 1, optimizing codon usage of a GFP reporter gene to reflect the codon bias of the C. reinhardtii chloroplast genome increased GFP accumulation by approximately eighty-fold, to 0.5% of soluble protein (see, also, 14).

As disclosed herein, human monoclonal antibodies and fragments thereof can be expressed in transgenic algae chloroplasts. A large single chain antibody gene was engineered in C. reinhardtii chloroplast codon bias, and utilized the C. reinhardtii chloroplast atpA or rbcL promoters and 5′ untranslated regions to drive expression. This antibody is directed against herpes simplex virus glycoprotein D (15), and contains the entire IgA heavy chain protein fused to the variable region of the light chain by a flexible linker peptide. The lsc antibody accumulates as a soluble protein in transgenic chloroplasts, and binds herpes virus proteins, as determined by ELISA assays. This large single chain antibody assembles into higher order structures (dimers), in vivo, and contains no obvious post-translational modifications, aside from the disulfide bonds associated with dimerization. These results demonstrate the utility of algae as an expression platform for complex recombinant proteins.

Methods

C. reinhardtii Strains, Transformation and Growth Conditions

All transformations were carried out on C. reinhardtii strain 137c (mt+) as described (14). Cultivation of C. reinhardtii transformants for expression of HSV8-LSC was carried out in TAP medium (19) at 23° C. under illumination and cell density.

Plasmid Construction

All DNA and RNA manipulations were carried out essentially as described (20; 21; see, also, Mayfield et al., Proc. Natl. Acad. Sci., USA 100:438-442, 2003, which is incorporated herein by reference). The coding region of the HSV8-lsc gene (SEQ ID NO:47) was synthesized de novo according to the method of (22) and as described (14). The resulting 1893 bp PCR product was cloned into plasmid pCR2.1 TOPO (Invitrogen Corp.) according to the manufacturers protocol. The alpA and rbcL promoters and 5′ UTR, and the rbcL 3′ UTR were generated via PCR (14).

Southern and Northern Blot Analysis

Southern blots and ³²P labeling of DNA for use as probes were carried out as described (20). Radioactive probes used on Southern blots included the 2.2 kb Bam HI/Pst I fragment of p322 (probe 5′ p322), the 2.0 kb Bam HI/Xho I fragment of p322 (probe 3′ p322) and the 1926 bp Nde I/Xba I fragments from HSV8-lsc. Additional radioactive probes used in northern blot analysis included the psbA cDNA. Northern and Southern blots were visualized utilizing a Packard Cyclone Storage Phosphor System equipped with Optiquant software.

Protein Expression, Western Blotting and ELISA

For Western blot analysis proteins were isolated from C. reinhardtii as described (14). Flag affinity purified C. reinhardtii HSV8-lsc were isolated in TRIS buffered saline (TBS; 25 mM TRIS ph 7.4, 150 mM NaCl) containing complete protease inhibitor cocktail tablets (Roche, Inc.) and phenylmethylsulfonyl fluoride MSF) at 1 mM final concentration. Extracts were purified using anti Flag M2 agarose beads (Sigma) according to the manufacturer's protocol. ELISA assays were carried out on volumes of 100 μl volumes in 96 well microtiter plates (Costar) coated with 100 μl of HSV proteins.

Samples for use in ELISA were diluted in blocking buffer comprised of phosphate buffered saline (PBS; 137 mM NaCl, 2.7 mM KCl, 1.8 mM K₂HPO₄, 10 mM Na₂HPO₄, pH 7.4) and 5% nonfat dry milk. Incubations were carried out for 8 hr at 4° C. with rocking. Plates were then rinsed with PBS plus 0.5% Tween 20 three times, then incubated with anti-Flag antibody for 8 hr at 4° C. Plates were again rinsed three times and incubated with alkaline phosphatase conjugated goat-anti-mouse antibody (Santa Cruz Biotechnology) for 8 hr at 4° C. Plates were once again rinsed three times with PBS plus 0.5% Tween 20 and developed using 100 μl p-nitrophenyl phosphate (pNPP, Sigma). Reactions were terminated via the addition of 50 μl 3 N NaOH.

Protein concentrations were determined using a BioRad Protein assay reagent. Western blots were carried out as described (23) using a murine anti-Flag primary antibody (Sigma) and an alkaline phosphatase conjugated goat anti-mouse secondary antibody (Santa Cruz Biotechnology).

Results

De Novo Synthesis of a Large Single Chain Antibody Gene in C. reinhardtii Chloroplast Using a Codon Bias Polynucleotide

To develop robust expression of recombinant antibodies in the C. reinhardtii chloroplast, a single chain antibody gene was synthesized using codons optimized to reflect abundantly translated C. reinhardtii chloroplast mRNAs. The engineered antibody was derived from a human antibody library displayed on phage, and identified by panning with herpes simplex virus proteins (15). This antibody, termed HSV8, was previously shown to bind the viral surface antigen glycoprotein D (16), and both Fab or IgG1 versions of this antibody act as efficient neutralizing antibodies, in vivo and in vitro (15, 16).

As simple scfv antibodies can be made in bacterial or yeast systems, an attempt was made to synthesize a more complex antibody in chloroplast, but one that could still be translated from a single mRNA. A single chain antibody was designed containing the entire heavy chain region fused to the variable region of the light chain gene via a flexible linker peptide. The primary amino acid sequence of this unique, large single chain (lsc) protein is shown as SEQ ID NO:48, which is encoded by SEQ ID NO:47.

Construction of a Chimeric C. reinhardtii Chloroplast Large Single Chain Antibody Gene

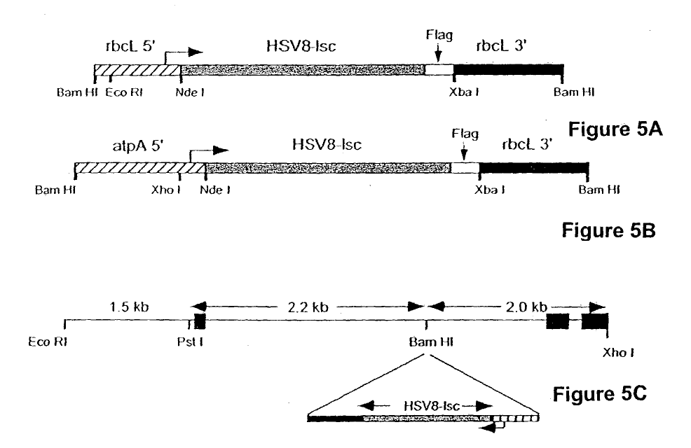

To generate transgenic chloroplast expressing the recombinant antibody, chimeric genes were generated containing either the atpA or rbcL promoter and 5′ UTR fused to the codon optimized HSV8-lsc coding region, followed by the rbcL 3′UTR (FIGS. 5A and 5B, respectively). Integration of genes into the chloroplast genome occurs by homologous recombination, and requires sequence homology between the transformation vector and the chloroplast genome (17). The C. reinhardtii chloroplast transformation vector p322 (14) was utilized. As diagrammed in FIG. 5B, the chimeric antibody genes were ligated into the Bam HI site of p322 to create plasmid p322/atpA-HSV8 and plasmid p322/rbcL-HSV8. The p322/HSV8 constructs were co-transformed into C. reinhardtii chloroplasts via particle bombardment (17), along with plasmid p228, containing a 16S ribosomal gene conferring spectinomycin resistance.

Southern Blot Analysis of HSV8-lsc Transgenic Chloroplast

Primary transformants were selected on media containing spectinomycin and screened by Southern blot analysis for HSV8 gene integration. HSV8 positive transformants were taken through additional rounds of selection to isolate homoplasmic lines in which all copies of the chloroplast genome contained the introduced HSV8-lsc gene. Two homoplasmic transformants were selected, one 10-6-3, containing the atpA promoter driving HSV8-lsc and the other, 20-4-4, containing the rbcL promoter driving HSV8-lsc. Genomic DNA from wt and the two HSV8-lsc transformants was digested with Eco RI and Xho I, separated on agarose gels, and subjected to Southern blot analysis. C. reinhardtii DNA was prepared as described in Example 3, digested with Eco RI and Xho I, and filters were hybridized with the radioactive probes indicated by the double arrowheads in FIG. 5C.

Hybridization with a ³²P labeled Nde I/Xba I fragment of the HSV8 coding region identified a 6.0 kb band in both the atpA-HSV8 and rbcL-HSV8 transgenic strains, while no detectable band was observed in the wt lane, as expected. When these same blots were hybridized with a ³²P labeled 1.5 kb Eco RI to Pst I fragment from the 5′ end of p322, a 5.7 kb fragment was visualized in the wt sample, while a slightly larger 6.0 kb fragment was identified in the two transgenic strains. Hybridization with a ³²P labeled Bam HI/Xho I fragment from the 3′ end of p322, resulted in the visualization of 2.5 kb and 2.0 kb in 10-6-3 and 20-4-4, respectively, while the wt strain again showed a band of 5.7 kb. These results demonstrate that the HSV8 gene had correctly integrated into the p322 silent site of the chloroplast genome, and that all copies of the chloroplast genome contained the HSV8 gene.

Accumulation of HSV8-lsc mRNA in Transgenic Strains

Chloroplast expressed HSV8-lsc mRNA in transgenic C. reinhardtii strains also was examined. Total RNA isolated from untransformed (wt), and atpA HSV8-lsc transformed (10.6.3), and rbcL transformed (20.4.4) strains was separated on denaturing agarose gels and blotted to nylon membrane. The membranes were either stained with methylene blue or hybridized with a psbA cDNA probe, or a hsv8 specific probe. Northern blot analysis of total RNA was used to determine if the HSV8 genes were transcribed in transgenic C. reinhardtii chloroplasts. Ten μg of total RNA from wt and the two transgenic lines was separated on denaturing agarose gels and blotted to nylon membrane. Duplicate filters were stained with methylene blue and hybridized with either a ³²P labeled psbA cDNA probe, or an HSV8 specific probe. Ribosomal RNA and psbA mRNA accumulate to similar levels in wt and each of the transgenic strains, demonstrating that equal amounts of RNA were loaded, and that introduction of the transgene does not alter endogenous mRNA accumulation. Hybridization with an HSV8 specific probe showed that strains 10-6-3 and 20-4-4 accumulate HSV8-lsc mRNA of the correct size, while no HSV8 signal was detected in the wt lane, as expected.

Analysis of HSV8-lsc Protein Accumulation in Transgenic C. reinhardtii Chloroplasts

HSV8-lsc antibody levels were measured by Western blot analysis using an anti-flag antibody to determine if HSV8-lsc protein accumulated in the transgenic lines. Twenty μg of total protein from an E. coli strain expressing HSV8-lsc from a pET vector, and 20 μg of total protein from C. reinhardtii wt and the two transgenic lines, was separated by SDS-PAGE and either stained with Coomassie blue, or subjected to western blot analysis with anti-Flag antisera. For bacterial expression, the Nde I/Bam HI fragment of codon optimized HSV8-lsc gene was ligated into a pET vector and expression was induced by addition of IPTG. The Coomassie stained gel indicated that equal amounts of protein were loaded in each lane, and that overall protein accumulation was normal in the transgenic lines. Western blot analysis of the same samples using an anti-Flag antibody showed a robust signal of the correct molecular weight in both of the HSV8-lsc transgenic strains and E. coli, but no signal in the C. reinhardtii wt lane, as expected.

Characterization of HSV8-lsc Antibodies Expressed in E. coli and Chloroplast

To ascertain if the HSV8-lsc that accumulated in C. reinhardtii chloroplast was functional, the chloroplast expressed protein was characterized along with that of the bacterial expressed HSV8-lsc. HSV8-lsc transgenic bacteria and algae were resuspended in TBS, and the cells lysed by sonication. Soluble proteins were separated from insoluble proteins by centrifugation. Equal amounts of protein from the soluble fractions and from the insoluble pellets were separated by SDS-PAGE, and HSV8-lsc proteins visualized by western blot analysis. Approximately 60% of the HSV8-lsc produced in bacteria partitioned to the insoluble fraction, while the HSV8-lsc produced in chloroplast was found exclusively in the soluble fraction.

To determine if chloroplast expressed antibodies contained any post-translational modifications, the antibodies were examined by SDS-PAGE and western blot analysis on reducing and non-reducing gels. Soluble proteins from C. reinhardtii transgenic line 10.6.3, were either treated with β-mercaptoethanol (+Bme) or without (no Bme) reducing agent prior to separation on SDS-PAGE. Proteins were blotted to nitrocellulose membrane and decorated with anti-flag antibody. Under non-reducing conditions any disulfide bonds formed between the two heavy chain moieties of the antibody should remain intact allowing the antibody to migrate as a larger species. Under non-reducing conditions chloroplast expressed HSV8-lsc runs as a much larger protein of approximately 140 kDa, the size expected of a dimmer. Treatment with Bme, to reduce disulfide bonds, results in the migration of the chloroplast HSV8-lsc proteins at the predicted molecular weight of the monomer at 68 kDa.

To ascertain if any other post-translational modifications might be present in the chloroplast expressed proteins, the bacterial and chloroplast expressed proteins was characterized by mass spectrometery. The mass spectra of peptide fragments from both the E. coli and chloroplast expressed protein had an almost identical pattern, indicating that no additional modifications are made to the chloroplast protein.

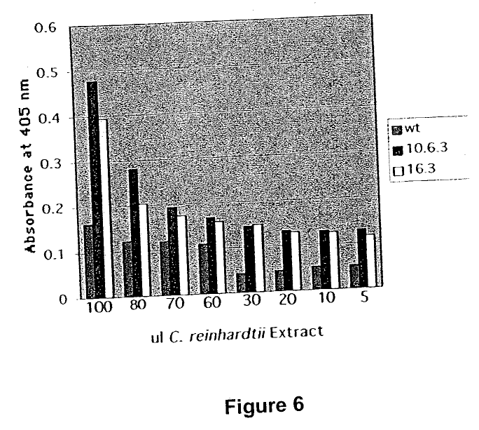

The ability of chloroplast expressed HSV8-lsc to bind HSV8 proteins was examined to confirm that the HSV8-lsc accumulating in the transgenic chloroplast was functional. HSV8-lsc was purified from transgenic chloroplast using an anti-flag affinity resin. As shown in FIG. 6, the chloroplast produced antibody recognized HSV8 proteins in ELISA assays in a robust manner.

Modulation of HSV8-lsc Accumulation in Transgenic Algae

The effect of different growth regimes on the accumulation of antibody in the two transgenic strains, 10-6-3 and 20-4-4 was examined to determine whether the expression of HSV8-lsc in C. reinhardtii chloroplast could be modulated. Cultures of each strain were maintained at 10⁶ or 10⁷ cells per ml and grown in either a 12/12 light-dark cycle (5000 lux), or under continuous light (5000 lux). Cells were harvested by centrifugation and 20 μg of soluble protein was resolved on SDS PAGE, and HSV8-lsc visualized by western blotting with anti Flag antibody.

Accumulation of HSV8-lsc varies considerably depending upon the growth conditions. Expression under the control of the rbcL promoter/5′UTR in strain 20-4-4 showed a marked increase in the accumulation of antibody at the end of the dark phase or immediately after entering the light phase, regardless of the cell density. In comparison, the alpA promoter/5′UTR in strain 10-6-3 directed fairly constant levels of HSV8-lsc production at 10⁶ cells per ml in a light-dark cycle, yet showed a tremendous increase in lsc accumulation upon entering the light phase when cell are cultured at 10⁷ cells per ml. When grown under continuous light, both strains exhibited higher accumulation at 10⁶ cells per ml than at 10⁷ cells per ml. These results demonstrate that accumulation of HSV8-lsc in chloroplast of C. reinhardtii can be optimized, dependent upon the light regime used to culture the cells, the phase in the cycle at which cells are harvested, and the promoter/UTR used to drive expression.

A human monoclonal antibody was expressed in the chloroplast of green algae. High levels of recombinant protein expression were achieved by optimizing the codon usage within the antibody coding sequence to reflect the codon usage of abundant chloroplast proteins, and by driving expression of the chimeric gene using the chloroplast atpA or rbcL promoters and 5′ UTRs. This large single chain (lsc) antibody contains the entire IgA heavy chain fused to the variable region of the light chain by a flexible linker, and accumulated as a fully soluble protein in chloroplast. The antibody was directed against glycoprotein D of herpes simplex virus, and the alga expressed antibody bound to herpes proteins as determined through ELISA. This lsc antibody contains the Fc portion of the heavy chain, which is the site normally involved in intermolecular disulfide bond formation leading to dimerization of the antibody. The chloroplast expressed antibody assembled into higher order complexes that are susceptible to reduction by Bme, indicating that the chloroplast expressed antibody forms dimers in vivo. Formation of disulfide bonds in recombinant proteins expressed in chloroplast has been shown for human somatotropin expressed in tobacco chloroplast (8), and was somewhat expected due to the presence of protein disulfide isomerase in algal chloroplasts (18). This lsc antibody also contains putative sites for N-linked glycosylation. Chloroplast encoded proteins are not known to be glycosylated and, indeed, there was no evidence of glycosylation of the chloroplast expressed antibody based upon mass spectral analysis.

The transgenic strains generated showed differential accumulation of antibody depending upon the promoter used to drive expression, as well as the cell density and light conditions under which they are cultured. The reasons for these large fluctuations in antibody accumulation likely arise from a variety of factors including stability and translational competence of the chimeric mRNAs, and turnover of the antibody protein. These results demonstrate that antibody accumulation can be positively impacted by growth conditions and indicate that high levels of antibody accumulation (exceeding 1% of soluble protein) can be achieved in alga simply by utilizing optimal growth conditions compatible with specific promoter and UTR combinations.

Recombinant proteins can be produced in a variety of protein expression systems. Complex therapeutic proteins, like monoclonal antibodies (mAbs), are primarily produced by culture of transgenic mammalian cells. Costs for mAb production in cultured mammalian cells averages approximately $150/gram for raw materials, while in plant systems mAb production has been estimated to cost $0.05/gram (1). Costs for production of mAbs in algal systems are expected to rival those in terrestrial plants, given that media costs for algae are quite reasonable ($0.002/liter). In addition, algae can be grown in continuous culture and their growth medium recycled.

Aside from the tremendous cost advantage of producing mAbs in algae, there are a number of specific attributes that make alga ideal candidates for recombinant protein production. First, transgenic algae can be generated quickly, requiring only a few weeks between the generation of initial transformants and their scale up to production volumes. Second, both the chloroplast and nuclear genome of algae can be genetically transformed, opening the possibility of producing a variety of transgenic proteins in a single organism, a requirement if multimeric protein complexes such as secretory antibodies are to be produced. In addition, algae have the ability to be grown on scales ranging from a few milliliters to 500,000 liters, in a cost effective manner. These attributes, and the fact that green algae fall into the GRAS (generally regarded as safe) category, make C. reinhardtii a particularly attractive alternative to other systems for the expression of recombinant proteins. Finally, while this example specifically addresses the production of antibodies in algae, this system should be amenable to the production of virtually any recombinant protein.

### REFERENCES CITED

Each of the following articles is incorporated herein by reference.

1. Dove, (2002) Nature Biotechnol. 20, 777-779

2. Motmans and Bouche, Antibodies: The Next Generation (2000) Report to Auerbach Grayson & Company, Inc.

3. Hiatt et al., (1989) Nature 342, 76-78

4. Ma et al., (1994) Eur. J Immunol. 24, 131-138

5. Ma et al., (1995) Science 268, 716-719

6. Ellstrand, (2001) Plant Physiol. 125, 1543-1545.

7. Quist and Chapela, (2001) Nature 414, 541-543.

8. Staub et al., (2000) Nature Biotechnol. 18, 333-338.

9. Kota et al., (1999) Proc. Natl. Acad. Sci. USA 96, 1840-1845.

10. Sidrov et al., (1999) Plant J. 19, 209-216.

11. Ruf et al., (2001) Nature Biotechnol. 19, 870-875.

12. Heifetz, (2000) Biochimie 82, 655-666

13. Harris, (1989) The Chlamydomonas Sourcebook Academic Press, Inc.

14. Franklin et al., (2002) Plant J. 30, 733-744.

15. Burioni et al., (1994) Proc. Natl. Acad. Sci. USA. 91, 355-359.

16. De Logu et al., (1998) J. Clin. Microbiol. 36, 3198-3204.

17. Boynton et al., (1988) Science 240, 1534-1538

18. Kim and Mayfield, (1997) Science 278, 1954-1957

19. Gorman et al., (1965) Proc. Natl. Acad. Sci. USA 54, 1665-1669.

20. Sambrook et al., (1989) Molecular Cloning. A Laboratory Manual Cold Spring Harbor Laboratory Press.

21. Cohen et al., (1998). Meth. Enzymol. 297, 192-208.

22. Stemmer et al., (1995) Gene 164, 49-53.

### EXAMPLE 4

### Expression of a Luciferase Fusion Protein from a Chloroplast Codon Biased Bacterial luxAB Gen

This Example confirms the robust expression in chloroplasts of a luciferase fusion protein encoded by a chloroplast codon biased synthetic polynucleotide.

Luciferase reporter genes have been successfully used in a variety of organisms to examine gene expression in living cells, but have yet to be successfully developed for use in chloroplast. As disclosed in Example 1, a green fluorescent protein (gfp) has been expressed from a chloroplast codon biased polynucleotide and was useful as a reporter of chloroplast gene expression. Since gfp can exhibit high auto-fluorescence, and relatively high levels of expression and gfp protein accumulation are required for visualization in chloroplast, a luciferase reporter protein encoded by a chloroplast codon biased polynucleotide was developed a luciferase reporter by synthesizing the two subunit bacterial luciferase, luxAB, as a single fusion protein in C. reinhardtii chloroplast codon bias. As disclosed herein, the chloroplast luciferase gene, luxCt, was expressed in C. reinhardtii chloroplasts under the control of the atpA promoter and 5′ UTR and rbcL 3′UTR. The luxCt is a sensitive reporter of chloroplast gene expression, allowing luciferase activity to be measured in vivo using a CCD camera or in vitro using a luminometer. Furthermore, luxCt protein accumulation, as measured by western blot analysis, is proportional to luminescence as determined both in vivo and in vitro. These results demonstrate the utility of the luxCt gene as a versatile and sensitive reporter of chloroplast gene expression in living cells.

Reporter genes have greatly enhanced the ability to monitor gene expression in a number of biological organisms. In chloroplasts of higher plants, β-glucuronidase (uidA, Staub and Maliga, 1993), neomycin phosphotransferase (nptII, Carrer et al., 1993), adenosyl-3-adenyltransferase (aadA, Svab and Maliga, 1993), and the gfp of Aequorea aequorea (Sidorov et al., 1999; Reed et al., 2001) have been used as reporter genes (Heifetz, 2000). Several reporter genes have also been expressed in the chloroplast of the eukaryotic green alga, C. reinhardtii, including aadA (Goldschmidt-Clermont, 1991; Zerges and Rochaix, 1994), uidA (Sakamoto et al., 1993, Ishikura et al., 1999), aphA6 (Bateman and Purton, 2000) and Renilla luciferase (Minko et al., 1999). Unfortunately, these initial reporter gene cassettes produced very low levels of protein accumulation, making them poor reporters for quantitative analysis of gene expression.

As disclosed in Example 1, high levels of reporter gene expression were obtained by optimizing codon usage of a gfp reporter gene (see, also, Franklin et al., 2002). A comparison of GFP accumulation in a strain of C. reinhardtii transformed with a non-optimized gfp and a strain transformed with the optimized cgfp revealed an eighty-fold increase in GFP accumulation from the cgfp gene in C. reinhardtii chloroplast. These results demonstrated that the previous inability to achieve high levels of reporter gene expression in the C. reinhardtii chloroplast was due to the codon bias utilized in C. reinhardtii chloroplast genes.

To extend the results obtained with gfp, and to obtain a reporter that could be visualized the cGFP in vivo, a bacterial luciferase gene was synthesized having C. reinhardtii chloroplast codon bias. The de novo synthesized lux gene was based on the bacterial luxAB gene of Vibrio harveyi (Baldwin et al., 1984, Johnson et al., 1986). The luciferase coding sequence was synthesized such that the luciferase A and B subunits were expressed as a single coding region by linking the A and B subunits with a flexible peptide linker (Kirchener et al., 1989, Olsson et al., 1989, Almashanu et al., 1990). The chloroplast optimized luciferase (luxCt) gene was placed in a cassette containing the atpA promoter and 5′UTR and the rbcL 3′ UTR. Transgenic lines containing the luxCt gene accumulated luxCt mRNA and LUXCt protein, as judged by northern and western blot analysis, respectively (see below). Luminescence from transgenic lines expressing luxCt was easily visualized with a CCD camera, when cells were treated with decanal, the bacterial luciferase substrate, while wt cells showed no luminescence in the same assays. Expression of luxCt, as judged by western blot analysis, was proportional to expression of luxCt, as judge by luminescence assays using a CCD camera, and by in vitro luminometer assays. Luciferase activity in transgenic lines could be measured over several orders of magnitude, demonstrating the sensitivity and utility of luxCt as a reporter of chloroplast gene expression in living cells.

Methods

C. reinhardtii Strains, Transformation and Growth Conditions

Transformations were carried out on C. reinhardtii strain 137c (mt+), or in the psbA deficient strain cc744 (REF). Cells were grown to late log phase (approximately 7 days) in the presence of 40 mM 5-fluorodeoxyuridine in TAP medium (Gorman and Levine, 1965) at 23° C. under constant illumination of 4,000 lux (high light) on a rotary shaker set at 100 rpm. Fifty ml of cells were harvested by centrifugation at 4,000×g at 4° C. for 5 min. The supernatant was decanted and cells resuspended in 4 ml TAP medium for subsequent chloroplast transformation by particle bombardment as described (Cohen et al., 1998). All transformations were carried out under spectinomycin selection (150 μg/ml) in which resistance was conferred by co-transformation with the spectinomycin resistance ribosomal gene of plasmid p228 (Chlamydomonas Stock Center, Duke University). Cultivation of C. reinhardtii transformants for expression of luxCt was carried out in TAP medium (Gorman and Levine, 1965) at 23° C. under constant illumination.

Plasmid Construction

DNA and RNA manipulations were carried out essentially as described in Sambrook et al. (1989) and Cohen et al. (1998). The coding region of the luxCt gene was synthesized de novo according to the method of Stemmer et al. (1995) from a pool of primers, each 40 nucleotides in length. The 5′ and 3′ terminal primers used in this assembly contained restriction sites for Nde I and Xba I, respectively. The resulting 2094 bp PCR product containing the luxCt gene was then cloned into the pCR2.1 TOPO plasmid (Invitrogen Corp.) according to the manufacturer's protocol to generate plasmids pluxCt. The atpA promoter and 5′ UTR and the rbcL 3′ UTR were generated as described (Mayfield et al., 2002). Chloroplast transformation plasmid p322 was constructed as described (Franklin et al., 2002).

Southern Blot and Northern Blot Analysis

Southern blots and ³²P labeling of DNA for use as probes were carried out as described (Sambrook et al., 1989; and Cohen et al., 1998). Radioactive probes used on Southern blots included the 2 kb Nde I/Xba I luxCt coding region (probe luxCt), and the 2.0 kb Bam HI/Xho I fragment of p322 (probe 3′ p322). A 0.9 kb Eco RI/Xba I luxCt probe was used to detect luxCt mRNA on northern blots. Additional radioactive probes used in northern blot analysis included the rbcL cDNA. Northern blots and Southern blots were visualized utilizing a Packard Cyclone Storage Phosphor System equipped with Optiquant software.

Protein Expression, Western Blot Analysis and Luminescence Assays

Plasmids pluxAB and pluxCt were transformed into E. coli strain BL21 and cells grown overnight in liquid media. For western blot analysis proteins were isolated from E. coli or from C. reinhardtii utilizing a buffer containing 750 mM Tris-Cl, pH 8.0, 15% sucrose (wt/vol), 100 mM Bme, 1 mM PMSF. Samples were centrifuged for 30 min at 13,000×g at 4° C. with the resulting supernatant used in western blot analysis. C. reinhardtii proteins for use in in vitro luminescence assays were prepared in 50 mM Na₂HPO₄, pH 7.0, 50 mM Bme, 400 mM sucrose buffer, and the crude lysate was centrifuged for 30 min at 13,000×g at 4° C. with the resulting supernatant used in luciferase assays. 96 well microtiter assays were adapted from the bacterial luciferase method (Langridge and Szalay, 1994). C. reinhardtii soluble proteins were diluted in luciferase extraction buffer to 100 μl per sample, to which 125 μl of 500 μM NADH in 50 mM Tris-Cl, pH 8.0, and 0.025 U of diaphorase in 50 mM Na₂HPO₄, 50 mM Bme, 1% bovine serum albumin buffer were added. To this resultant mixture, 130 μl of a solution containing 125 μl 100 μM FMN⁻ in 200 mM Tricine, pH 7.0 and 5 μl 0.1% decanal sonicated for 10s in 50 mM Na₂HPO₄, pH 7.4 was added. Photon measurement in relative light units (rlu) began 5 seconds after FMN⁻/decanal addition with a LJL Biosystems Analyst AD luminometer (fluorescence reader) equipped with Criterion Host software. Protein concentrations were determined using the BioRad Protein assay reagent.

Western blots were carried out as described by Cohen et al. (1998) using a rabbit anti-luxAB primary antibody (REF) and an alkaline phosphatase labeled goat anti-rabbit secondary antibody (Sigma). Colony luminescence was imaged with a Berthold Night Owl CCD camera, model LB 981, equipped with 700 nm emission filter to block chlorophyll fluorescence (Chroma Corp.). Exposure times of 30 sec to 5 min were sufficient to visualize luciferase luminescence in most cases. Images were generated using WinLight software.

Results

De Novo Synthesis of a luxAB Gene in C. reinhardtii Chloroplast Codon Bias

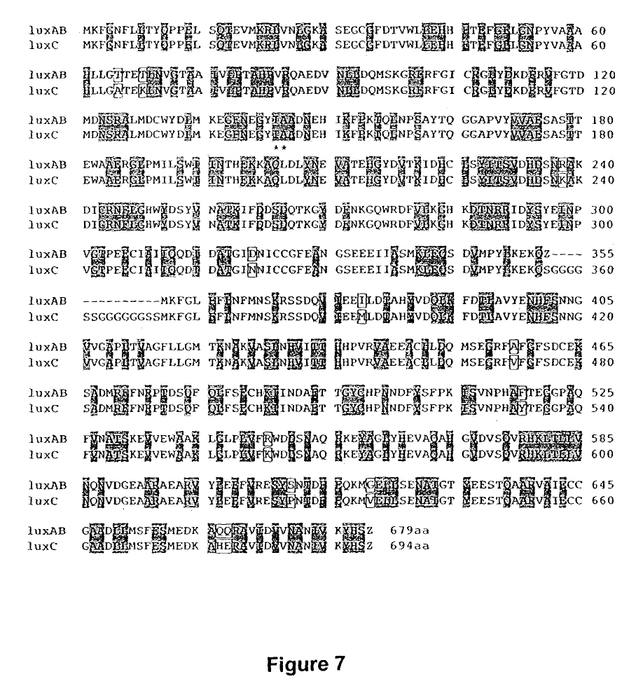

To develop a sensitive reporter of gene expression in chloroplast, a luciferase gene was synthesized using codons optimized to reflect abundantly expressed genes of the C. reinhardtii chloroplast (Example 1; Franklin et al., 2002). The luciferase gene, luxCt (FIG. 7, was designed based on the bacterial luciferase AB gene of Vibrio harveyi (luxAB; Baldwin et al., 1984). For chloroplast expression, the two subunits of luxAB were linked into a single coding sequence by eliminating the stop codon of the A subunit and linking the B subunit, in the correct reading frame, with a flexible peptide sequence to create a single fusion protein (FIG. 7). The V harveyi luxAB sequence was obtained from the GenBank database and a series of oligonucleotides were designed based on the amino acid sequence, but changing codon usage to reflect those of highly expressed C. reinhardtii chloroplast genes. The gene was assembled by the method of Stemmer et al. (1995). PCR products were cloned into E. coli plasmids, the synthetic gene sequenced, and errors corrected by site directed mutagenesis. An Nde I site was placed at the initiation codon and an Xba I site placed immediately downstream of the stop codon, for ease in subsequent cloning. The resulting gene, luxCt, was cloned into an E. coli expression cassette and luciferase expression was assayed by luminescence imaging with a CCD camera. Surprisingly, no luminescence was detected in bacteria containing the luxCt gene, although high luminescence could be detected in bacteria transformed with the bacterial luxAB gene (Kondo et al., 1993).

To ensure that a mutation had not inadvertently been introduced into the luxCt gene during cloning into the E. coli vector, both the luxCt and the bacterial luxAB genes contained in the E. coli expression plasmids were sequenced. No errors were detected in the luxCt gene compared to the desired sequence, but a number of differences were identified in the luxAB sequence from the plasmid used to express luxAB in bacteria (Kondo et al., 1993), and the luxAB sequence reported in the GenBank database (Acc. No. E12410). Alignment of luxAB proteins from several different bacterial species (Johnson et al. 1990) with the synthetic luxCt protein identified a number of differences in amino acid sequence, but only one of these differences was in a conserved amino acid. Therefore, site directed mutagenesis was used to restore a conserved glutamate at position 204, and an adjacent leucine at position 205. No other amino acid was changed, as none was conserved among the set of luxAB proteins surveyed.

The luxCt Fusion Protein Gene Produces a Functional Luciferase in Bacteria

To determine if the synthetic luxCt gene was capable of producing functional luciferase, luminescence was measured in E. coli cells transformed with an expression plasmid containing either the luxAB or the luxCt gene. Western blot analysis was performed using crude E. coli lysates from cells expressing either the luxAB or luxCt gene; 20 μl were subjecting to SDS PAGE, and blotted to nitrocellulose. The blots were decorated with anti-luxAB primary antibody followed by anti-rabbit secondary coupled with alkaline phosphatase, and the protein visualized by alkaline phosphatase activity staining. The alpha (A) and beta (B) subunits of luxAB were identified, as was the single fusion protein (FP) of luxCt. In addition, luciferase expression was determined in E. coli was grown overnight on agar media and treated with decanol vapor. Untransformed E. coli cells or cells expressing either the luxAB or luxCt genes were photographed with reflect light (photograph), or visualized by luminescent imaging with a CCD camera (luminescence). When E. coli cells were treated with decanal and imaged with a CCD camera, both luciferase strains luminesced, while untransformed E. coli showed no light signal, as expected. Western blot analysis, using a polyclonal antibody raised against native luxAB protein, showed a signal for both the A and B subunits of the bacterial luciferase protein in the luxAB strain, and a single band corresponding to the fused protein in the luxCt strain. The A and B proteins of luxAB accumulated to higher levels in bacteria than the single fusion protein of luxCt, while the luminescent signal for these proteins, 2:1 luxAB:luxCt, was approximately proportional to luciferase protein accumulation.

Construction of a luxCt Expression Cassette and Southern Blot Analysis of luxCt Transformants

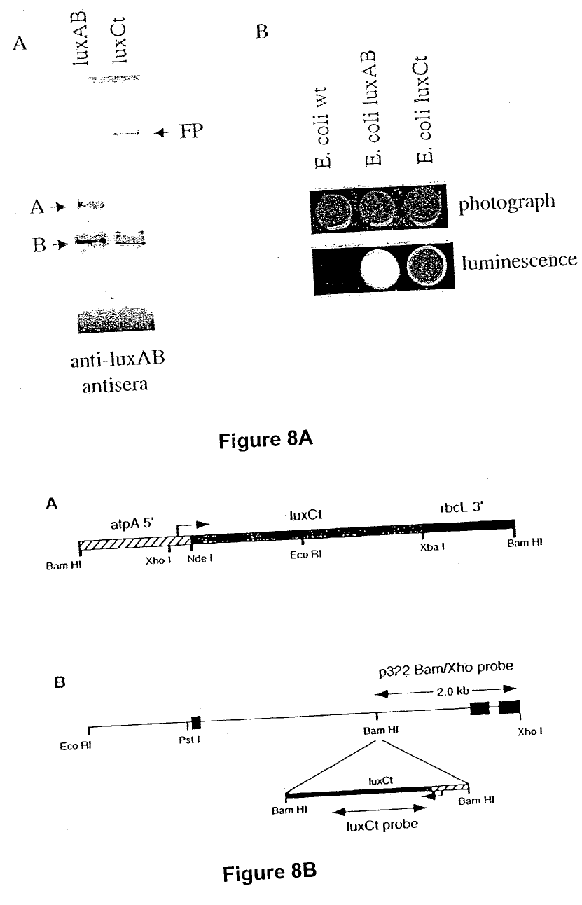

Upon demonstrating that the luxCt coding sequence produced a functional luciferase, C. reinhardtii chloroplasts were transformed with a luxCt cassette. For luciferase expression in chloroplast, the expression cassette shown in FIG. 8 was constructed. The luxCt coding sequence was ligated down stream of the atpA promoter and 5′ UTR, and upstream of the rbcL 3′ UTR (FIG. 8A). The chimeric atpA/luxCt gene was then ligated into chloroplast transformation plasmid p322 at the unique Bam HI site to create plasmid p322-atpA/luxCt (FIG. 8B).

Wild type C. reinhardtii cells were transformed with the p322-atpA/luxCt plasmid and the selectable marker plasmid p228, conferring resistance to spectinomycin. Primary transformants were screened for the presence of the luxCt gene by luminescent assays on the CCD camera, and positive transformants were confirmed by Southern blot analysis. Transformants were taken through additional rounds of selection to isolate homoplasmic lines in which all copies of the chloroplast genome contained the introduced luxCi gene.

Two homoplasmic luxCi transformants, 10.6 and 11.5, were selected for further analysis. FIG. 8 shows the luxCt constructs with relevant restriction sites indicated. Correct integration of the 8.7 kb Eco/Xho region of plasmid p322-atpA/luxCt into the chloroplast genome was ascertained using either the Nde I-Xba I fragment of luxCt, or the Bam HI-Xho I fragment of plasmid p322, as indicated in FIG. 8. Southern blot analysis of luxCt C. reinhardtii chloroplast transformants was performed. C. reinhardtii DNA was prepared as described in Example 4, digested simultaneously with Eco RI and Xho I and subjected to Southern blot analysis. Filters were hybridized with the radioactive probe indicated in FIG. 8B. The two transformants contained luxCt hybridizing bands, while the wild type strain showed no signal with this luxCt coding region probe. Two bands were identified in the transgenic lines because the luxCt gene contains a single Eco RI site in the middle of the gene. Hybridization with the Bam HI-Xho I fragment from the p322 plasmid identified a single band in wt and a different sized band in the two transformants, as expected. Each of these bands was of the correct predicted size for both the wt and transformant lines. These results demonstrate that the two transgenic lines are homoplasmic.

Accumulation of luxCt mRNA in Transgenic Strains

Northern blot analysis of total RNA was used to confirm that the luxCt gene was transcribed in transgenic C. reinhardtii chloroplasts. Ten μg of total RNA, isolated from wt and the two transgenic lines, was separated on denaturing agarose gels and blotted to nylon membrane. Duplicate filters were stained with methylene blue, or hybridized with a ³²P labeled luxCt probe, or an rbcL cDNA probe. Each of the strains accumulated rRNA (stained bands) and rbcL mRNA to similar levels, demonstrating that equal amounts of RNA were loaded for each lane, and that chloroplast transcription and mRNA accumulation are normal in the transgenic lines. Hybridization of the filters with the luxCt specific probe showed that both transgenic lines accumulate luxCi mRNA of the predicted size, while no luxCt signal was observed in wt cells, as expected.

Analysis of luxCt Protein Accumulation in Transgenic C. reinlhardtii Chloroplasts

Western blot analysis was used to confirm that luxCt protein accumulated in the transgenic lines. Twenty μg of total soluble protein (tsp) from wt and the two transgenic lines was separated by SDS-PAGE and either stained with Coomassie, or subjected to western blot analysis. The Coomassie stained gel indicated that equal amounts of protein were loaded in each lane, and that the transgenic lines accumulated a similar set of proteins as compared to wt. Western blot analysis of the same samples identified a single band corresponding to the fusion protein in both of the luxCt transgenic lanes. No signal was observed in the wt C. reinhardtii lane, as expected.

Use of luxCt as a Reporter of Chloroplast Gene Expression in Living Cells

To ascertain the utility of the luxCt gene as a reporter of chloroplast gene expression in living C. reinhardtii cells, luminescence was measured for wt and transgenic cells grown on agar plates. Cells were plated on solid media and maintained for seven days under continuous light (1000 lux). Decanal, the substrate for luxAB, was swabbed onto the Petri plate lids, and the plates were placed under a CCD camera. The transgenic lines appeared similar to wt cells when visualized under ambient light. Imaging with the CCD camera, after 5 min of dark adaptation to eliminate chlorophyll fluorescence, showed a bright luminescent signal for the two transgenic lines, and no signal for the wt strain. The signal from the luxCt transgenic lines was sufficient to visualize even small individual colonies in vivo.

In addition to transforming the luxCt cassette into wt (137c) cells, the cassette was transformed into a psbA deficient strain of C. reinhardtii (cc744, Chlamydomonas Genetics Center, http://www.botany.duke.edu/chlamy/). Again, primary transformants were screened by luminescent assays with the CCD camera, and positive transgenic lines were taken through several rounds of selection to obtain homoplasmic lines. Luminescence from the cc744/luxCt strain was much higher than from the 137c/luxCt strain. To identify if this increased luminescence was directly related to increased luciferase accumulation, luxCt protein accumulation and luciferase activity were measured in the 137c and cc744 transgenic lines. Wild type and luxCt transgenic lines luxCt137c and luxCtcc744 were grown on agar plates and treated with decanal. Cells were either photographed under reflective light (photograph), or visualized on a CCD camera (luminescence). Proteins were extracted from the cells and subjected to western blot analysis (western anti-luxAB) or quantitated by luminometer assays (luminometer). Western blot analysis revealed that approximately 10 fold more luxAB protein accumulated in the cc744 line compared to the 137c line, when cells were grown on solid TAP media in light. CCD camera luminescence assays revealed a similar increased signal in the cc744 line compared to the 137c line. Quantitation of luciferase activity using a luminometer revealed that the cc744-luxCt line had approximately 11 fold more luciferase activity than the 137c-luxCt line. These results demonstrate that the luxCt gene is a robust reporter of chloroplast gene expression, and that measurement of lux activity in vivo corresponded to luciferase accumulation as measured by both western blot analysis and in vitro luminescence assays.

Several heterologous genes have been employed as reporters of chloroplast gene expression, but their utility has been limited by poor sensitivity or an inability to be visualized in vivo. Luciferases have been used in a number of organisms as reporter genes (Greer and Szalay, 2002; Langeridge et al., 1994; Kondo et al., 1993; Kay, 1993) due to their high level of sensitivity and because luciferase can be readily visualized in living cells with little perturbation of the organism. This Example demonstrates the construction of a luciferase reporter gene for chloroplast expression by synthesizing the two subunit bacterial luciferase, luxAB, as a single fusion protein, and by optimizing the codon usage of this synthetic luciferase gene to reflect highly expressed genes from the C. reinhardtii chloroplast.

This Example extends the results of Example 1, which demonstrated that codon usage dramatically effected the expression of heterologous proteins in C. reinhardtii chloroplast by synthesizing a gfp in chloroplast-optimized codons (see, also, Franklin et al., 2002). The cgfp accumulated to 0.5% of total soluble protein within transgenic chloroplast, and could be visualized by fluorescent analysis of chloroplast extracts. However, even that relatively high level of GFP accumulation was insufficient to visualize the reporter in vivo. Using a mitochondrial optimized gfp gene, Komine et al reported visualization of gfp in transgenic C. reinhardtii chloroplast using fluorescence microscopy (Komine et al., 2002). However, very low levels of GFP protein accumulation were reported for the transgenic lines, and the fluorescence output in the mGFP strains did not appear to be above the background fluorescence in untransformed strains.

Based on the success of the chloroplast optimized gfp in improving protein accumulation, coupled with the fact that even relatively high levels of GFP could not be visualized in chloroplast in vivo, a luciferase gene was synthesized in chloroplast optimized codons to obtain a sensitive vital reporter. Expression of this codon optimized luxCt gene, placed under the control of the C. reinhardtii chloroplast atpA S promoter and UTR and the rbcL 3′ UTR, showed that the luxCt mRNA and luxCi protein accumulated in transgenic C. reinhardtii chloroplasts. Furthermore, the transgenic strains expressing luxCt accumulated sufficient levels of luciferase to be easily visualized by luminescence assays in vivo using a CCD camera. LuxCt protein accumulation, as measured by western blot analysis, was proportional to luciferase activity as measured by CCD camera luminescence assays or in vitro luminometer assays.

C. reinhardtii has been referred to as “green yeast”, a well deserved term given the excellent genetic characteristic of this organism that have allowed its use to dissect a number of cellular processes, most notably in the biogenesis of flagella and the photosynthetic apparatus. What has clearly been lacking, however, is a facile means to assay gene expression, especially in the chloroplast. The present results demonstrate the utility of the optimized luxCt gene as a reporter of chloroplast gene expression in vivo. The present results also demonstrate that luxCt is a sensitive reporter capable of monitoring gene expression even in small colonies of cells, making luxCt the reporter of choice for any high throughput analysis of chloroplast gene expression.

### REFERENCES CITED

Each of the following articles is incorporated herein by reference.

Almashanu et al., J. Biolumin. Chemilumin. 5:89-97, 1990.

Bateman and Purton, Mol. Gen Genet. 263:404-410, 2000.

Baldwin et al., Biochemistry 23:3663-7, 1984.

Carrer et al., Mol. Gen. Genet. 241:49-56, 1993.

Cohen et al., Meth. Enzymol. 297, 192-208, 1998.

Franklin et al., Plant J. 30:733-44, 2002.

Goldschmidt-Clermont, Nucl. Acids Res. 19:4083-4089, 1991.

Greer and Szalay, Luminescence 17:43-74, 2002.

Gorman and Levine, Proc. Natl. Acad. Sci. USA 54:1665-1669, 1965.

Heifetz, Biochimie 82:655-666, 2000.

Ishikura et al., J. Biosci. Bioeng. 87:307-314, 1999.

Johnston et al., J. Biol. Chem. 261:4805-11, 1986.

Kirchner et al., Gene 81:349-54, 1989.

Komine et al., Proc. Natl. Acad. Sci. USA 19:4085-90, 2000.

Kondo et al., Proc. Natl. Acad. Sci. USA 90:5672-5676, 1993.

Langridge et al., J. Biolumin. Chemilumin. 9:185-200, 1994.

Minko et al., Mol. Gen. Genet. 262:421-425, 1999.

Nakamura et al., Nucl. Acids Res. 27:292, 1999.

Olsson et al., Gene 81:335-47, 1989.

Reed et al., Plant J. 27:257-265, 2001.

Sambrook et al., Molecular Cloning: A Laboratory Manual (Cold Spring Harbor Laboratory Press 1989).

Sakamoto et al., Proc. Natl. Acad. Sci. USA 90:497-501, 1993.

Sidrov et al., Plant J 25:209-216, 1999.

Staub and Maliga, EMBO J. 12:601-606, 1993.

Stemmer et al., Gene. 164:49-53, 1995.

Svab and Maliga, Proc. Natl. Acad. Sci. USA 913-917, 1993.

Zerges and Rochaix, Mol. Cell. Biol. 14:5268-5277, 1994.

Although the invention has been described with reference to the above examples, it will be understood that modifications and variations are encompassed within the spirit and scope of the invention. Accordingly, the invention is limited only by the following claims.

What is claimed is:

1. A method of producing a polypeptide in a plastid, the method comprising introducing a first recombinant nucleic acid molecule into the plastid,
wherein the first recombinant nucleic acid molecule comprises
a first polynucleotide, which encodes at least one polypeptide,
operatively linked to a second polynucleotide, which comprises a first nucleotide sequence encoding a first ribosome binding sequence (RBS) operatively linked to a second nucleotide sequence encoding a second RBS,
wherein the first RBS can direct translation of the polypeptide in a prokaryote and the second RBS can direct translation of the polypeptide in a plastid,
under conditions that allow expression of the at least one polypeptide, thereby producing the polypeptide in the plastid.

2. The method of claim 1, wherein the first polynucleotide encodes a first polypeptide and at least a second polypeptide.

3. The method of claim 2, wherein the first polypeptide and at least second polypeptide comprise a fusion protein.

4. The method of claim 3, wherein the fusion protein comprises a single chain antibody.

5. The method of claim 4, wherein the first polynucleotide comprises SEQ ID NO:13 or a nucleotide sequence encoding SEQ ID NO:14.

6. The method of claim 1, wherein the plastid is a chloroplast.

7. The method of claim 6, wherein codons of the first polynucleotide are biased to reflect chloroplast codon usage.

8. The method of claim 7, wherein the first polynucleotide encodes a reporter protein or a mutant or variant thereof.

9. The method of claim 8, wherein the reporter protein is a green fluorescent protein.

10. The method of claim 9, wherein the first polynucleotide comprises SEQ ID NO:15, a nucleotide sequence encoding SEQ ID NO:1 or a nucleotide sequence encoding SEQ ID NO:2.

11. The method of claim 7, wherein the first polynucleotide sequence encodes a first polypeptide and at least a second polypeptide.

12. The method of claim 11, wherein the first polypeptide and at least second polypeptide comprise a fusion protein.

13. The method of claim 11, wherein the first polypeptide and second polypeptide comprise subunits of a protein complex.

14. The method of claim 13, wherein the protein complex is a heterodimer.

15. The method of claim 13, wherein the protein complex comprises a reporter protein.

16. The method of claim 15, wherein the reporter protein comprises a luciferase or a mutant or variant thereof.

17. The method of claim 16, wherein the luciferase comprises a bacterial luxAB gene product.

18. The method of claim 17, wherein the first polynucleotide comprises SEQ ID NO:45 or a nucleotide sequence encoding SEQ ID NO:46.

19. The method of claim 11, wherein the first polypeptide comprises an immunoglobulin heavy chain or a variable region thereof, and the second polypeptide comprises an immunoglobulin light chain or a variable region thereof.

20. The method of claim 19, wherein the first polypeptide and the second polypeptide comprise a fusion protein, thereby producing a single chain antibody.

21. The method of claim 20, wherein the first polynucleotide comprises SEQ ID NO:15; a nucleotide sequence encoding SEQ ID NO:16; SEQ ID NO:42; a nucleotide sequence encoding SEQ ID NO:43; SEQ ID NO:47, or a nucleotide sequence encoding SEQ ID NO:48.

22. The method of claim 1, further comprising introducing at least a second recombinant nucleic acid molecule into the plastid.

23. The method of claim 22, wherein the plastid is a chloroplast.

24. The method of claim 23, wherein the second recombinant nucleic acid molecule comprises a first polynucleotide, which encodes at least one polypeptide, operatively linked to a second polynucleotide, which comprises a first nucleotide sequence encoding a first ribosome binding sequence (RBS) operatively linked to a second nucleotide sequence encoding a second RBS, wherein the first RBS can direct translation of the polypeptide in a prokaryote and the second RBS can direct translation of the polypeptide in a chloroplast.

25. The method of claim 24, wherein the first recombinant nucleic acid molecule and the second recombinant nucleic acid molecule are co-expressed in the chloroplast.

26. The method of claim 1, wherein the first recombinant nucleic acid molecule is contained in a vector.

27. The method of claim 26, wherein the plastid is a chloroplast.

28. The method of claim 27, wherein the vector is a chloroplast vector, which comprises a nucleotide sequence of chloroplast genomic deoxyribonucleic acid (DNA) that can undergo homologous recombination with chloroplast genomic DNA.

29. The method of claim 28, wherein the vector further comprises a prokaryote origin of replication.

30. The method of claim 1, further comprising isolating the polypeptide from the plastid.

31. An isolated polypeptide obtained by the method of claim 30.

32. The isolated polypeptide of claim 31, comprising SEQ ID NO:2, SEQ ID NO:16, SEQ ID NO:43, SEQ ID NO:46, or SEQ ID NO:48.

33. An isolated ribonucleotide sequence, comprising a first ribosome binding sequence (RBS) operatively linked to a second RBS, wherein the first RBS and second RBS are spaced apart by about 5 to 25 nucleotides.

34. The ribonucleotide sequence of claim 33, wherein the first RBS and second RBS are spaced apart by about 10 to 20 nucleotides.

35. The ribonucleotide sequence of claim 33, wherein the first RBS and second RBS are spaced apart by about 15 nucleotides.

36. The ribonucleotide sequence of claim 33, wherein each of the first RBS and the second RBS independently consists of about 3 to 9 nucleotides.

37. The ribonucleotide sequence of claim 33, wherein each of the first RBS and the second RBS independently consists of about 4 to 7 nucleotides.

38. The ribonucleotide sequence of claim 33, wherein the first RBS or the second RBS or both comprises GGAG.

39. The ribonucleotide sequence of claim 33, wherein the second RBS further comprises a 5′-untranslated region (5′UTR) of a chloroplast gene.

40. The ribonucleotide sequence of claim 39, wherein the 5′UTR is encoded by a nucleotide sequence as set forth in any of SEQ ID NOS:4 to 8.

41. The ribonucleotide sequence of claim 39, wherein the chloroplast gene encodes a soluble protein.

42. The ribonucleotide sequence of claim 33, which is operatively linked to an initiator AUG codon.

43. The ribonucleotide sequence of claim 42, wherein the initiator AUG codon further comprises a Kozak sequence.

44. The ribonucleotide sequence of claim 43, wherein the initiator AUG codon further comprising a Kozak sequence is ACCAUGG.

45. The ribonucleotide sequence of claim 33, which is operatively linked to a polynucleotide encoding a polypeptide.

46. The ribonucleotide sequence of claim 45, wherein the polynucleotide comprises an initiator AUG codon.

47. The ribonucleotide sequence of claim 33, which consists of about 11 to 50 ribonucleotides.

48. The ribonucleotide sequence of claim 33, which consists of about 15 to 40 ribonucleotides.

49. The ribonucleotide sequence of claim 33, which consists of about 20 to 30 ribonucleotides.

50. The ribonucleotide of claim 33, further comprising an operatively linked polynucleotide encoding a polypeptide, whereby the first RBS directs translation of the polypeptide in a prokaryote and the second RBS directs translation of the polypeptide in a chloroplast.

51. A polynucleotide encoding the ribonucleotide sequence of claim 33.

52. The polynucleotide of claim 51, which comprises an initiator ATG codon operatively linked to the nucleotide sequence encoding the first RBS and second RBS.

53. The polynucleotide of claim 51, which comprises a cloning site positioned to allow operative linkage of an expressible polynucleotide to the first RBS and second RBS.

54. The polynucleotide of claim 53, wherein the cloning site comprises at least one restriction endonuclease recognition site, or at least one recombinase recognition site, or a combination thereof.

55. The polynucleotide of claim 51, which is flanked by a first cloning site and a second cloning site.

56. The polynucleotide of claim 55, wherein the first cloning site and the second cloning site are different.

57. The polynucleotide of claim 51, which is operatively linked to an expressible polynucleotide.

58. The polynucleotide of claim 57, wherein the expressible polynucleotide encodes at least a first polypeptide.

59. The polynucleotide of claim 58, wherein the expressible polynucleotide encodes the first polypeptide and at least a second polypeptide.

60. The polynucleotide of claim 59, wherein the expressible polynucleotide encodes the first polypeptide and a second polypeptide.

61. The polynucleotide of claim 60, wherein the first polypeptide and the second polypeptide are different.

62. The polynucleotide of claim 60, wherein the first polypeptide and second polypeptide comprise a fusion protein.

63. The polynucleotide of claim 62, wherein the expressible polynucleotide comprises a nucleotide sequence as set forth in SEQ ID NO:13, SEQ ID NO:15, SEQ ID NO:42, SEQ ID NO:45, or SEQ ID NO:47.

64. The polynucleotide of claim 60, further comprising a nucleotide sequence encoding an internal ribosome entry site, which is operatively linked between the coding sequence of the first polypeptide and the coding sequence of the second polypeptide.

65. The polynucleotide of claim 51, which is double stranded.

66. The polynucleotide of claim 65, which comprises, in operative linkage in a 5′ to 3′ orientation, a nucleotide sequence encoding the second RBS, a nucleotide sequence encoding the first RBS, and an initiator ATG; or a nucleotide sequence complementary to said polynucleotide.

67. The polynucleotide of claim 66, further comprising at least one cloning site positioned 3′ of the initiator ATG codon.

68. The polynucleotide of claim 65, which comprises, in a 5′ to 3′ orientation, a nucleotide sequence encoding the second RBS, a nucleotide sequence encoding the first RBS, and at least one cloning site positioned about 3 to 10 nucleotides 3′ of the nucleotide sequence encoding the first RBS; or a nucleotide sequence complementary to said polynucleotide.

69. The polynucleotide of claim 65, which is flanked at each end by at least one cloning site.

70. A vector, comprising the polynucleotide of claim 51 and a nucleotide sequence of chloroplast genomic deoxyribonucleic acid (DNA), wherein said nucleotide sequence can undergo homologous recombination with chloroplast genomic DNA.

71. The vector of claim 70, further comprising a cloning site positioned to allow operative linkage of at least one heterologous polynucleotide to the first RBS and second RBS.

72. The vector of claim 70, further comprising a prokaryote origin of replication.

73. The vector of claim 72, wherein the origin of replication is an E. coli origin of replication.

74. The vector of claim 70, wherein the nucleotide sequence of chloroplast genomic DNA comprises a first end and a second end.

75. The vector of claim 74, wherein the first end or the second end or both comprises at least one cloning site, or a cleavage product thereof.

76. The vector of claim 70, which is circularized.

77. The vector of claim 70, further comprising an initiator ATG codon operatively linked to the first RBS and second RBS.

78. The vector of claim 77, further comprising a cloning site positioned to allow operative linkage of at least one heterologous polynucleotide to the ATG codon.

79. The vector of claim 72, further comprising an expressible polynucleotide operatively linked to first RBS and second RBS.

80. The vector of claim 79, wherein the expressible polynucleotide comprises SEQ ID NO: 1, a nucleotide sequence encoding SEQ ID NO:2, SEQ ID NO:45, a nucleotide sequence encoding SEQ ID NO:46, or a combination thereof.

81. A cell, comprising the polynucleotide of claim 51.

82. The cell of claim 81, which is a plant cell.

83. The plant cell of claim 82, wherein the polynucleotide is in a chloroplast.

84. The plant cell of claim 83, wherein the polynucleotide is operatively linked to an expressible polynucleotide.

85. The plant cell of claim 83, wherein the expressible polynucleotide encodes at least a first polypeptide.

86. The plant cell of claim 85, wherein the expressible polynucleotide encodes the first polypeptide and at least a second polypeptide.

87. The plant cell of claim 85, wherein the expressible polynucleotide encodes the first polypeptide and a second polypeptide.

88. The plant cell of claim 87, wherein the first polypeptide and the second polypeptide are different.

89. The plant cell of claim 87, wherein the first polypeptide and second polypeptide comprise a fusion protein.

90. The plant cell of claim 84, wherein the expressible polynucleotide comprises a nucleotide sequence as set forth in SEQ ID NO:13, SEQ ID NO:15, SEQ ID NO:43, SEQ ID NO:45, SEQ ID NO:47, or a combination thereof.

91. A transgenic plant, comprising the plant cell of claim 83.

92. A plant cell or tissue obtained from the transgenic plant of claim 81.

93. A cutting of the transgenic plant of claim 92.

94. A seed produced by the transgenic plant of claim 92.

95. A cDNA or chloroplast genomic DNA library prepared from the transgenic plant of claim 91, or from a plant cell or plant tissue obtained from said transgenic plant.

96. The transgenic plant of claim 91, wherein the plant is an algae.

97. The transgenic plant of claim 91, wherein the plant is an angiosperm.

98. The transgenic plant of claim 97, wherein the angiosperm is a cereal plant, a leguminous plant, an oilseed plant, or a hardwood tree.

99. The transgenic plant of claim 91, wherein the plant is an ornamental plant.

100. A composition, comprising plant material obtained from the transgenic plant of claim 91.

101. The composition of claim 100, wherein the polynucleotide in the chloroplast is operatively linked to an expressible polynucleotide.

102. The composition of claim 101, wherein the expressible polynucleotide is biased for chloroplast codon usage.

103. The composition of claim 101, wherein the expressible polynucleotide encodes an antibody, or an antigen binding fragment thereof.

104. The composition of claim 103, which is in a form suitable for administration to a subject.

105. The composition of claim 104, wherein the subject is a mammal.

106. The composition of claim 104, wherein the subject is a human.

107. A chloroplast/prokaryote shuttle vector,
a nucleotide sequence of chloroplast genomic DNA, which can undergo homologous recombination with chloroplast genomic DNA;
a prokaryotic origin; and
a first ribosome binding sequence (RBS) operatively linked to a second RBS, wherein the first RBS can direct translation of an operatively linked expressible polynucleotide in a chloroplast, and the second RBS can direct translation of the operatively linked expressible polynucleotide in a prokaryote.

108. The shuttle vector of claim 107, further comprising a cloning site, wherein the cloning site is positioned such that a heterologous polynucleotide can be inserted into and operatively linked to the first RBS and the second RBS.

109. The shuttle vector of claim 107, further comprising an operatively linked expressible polynucleotide.

110. The shuttle vector of claim 109, wherein the expressible polynucleotide comprises a chloroplast codon biased polynucleotide.

111. An isolated polynucleotide encoding a protein or a mutant or variant thereof, wherein codons of the polynucleotide are biased to reflect chloroplast codon usage.

112. The polynucleotide of claim 111, which comprises a deoxyribonucleotide sequence.

113. The polynucleotide of claim 111, wherein the codons are biased to contain an adenine or a thymine at position three.

114. The polynucleotide of claim 111, which is flanked by a first cloning site and a second cloning site.

115. The polynucleotide of claim 111, wherein the protein comprises a fusion protein.

116. The polynucleotide of claim 111, wherein the protein is a reporter protein.

117. The polynucleotide of claim 116, wherein the reporter protein is a green fluorescent protein or a luciferase.

118. The polynucleotide of claim 117, comprising SEQ ID NO: 1, a nucleotide sequence encoding SEQ ID NO:2, SEQ ID NO:45, or a nucleotide sequence encoding SEQ ID NO:46.

119. The polynucleotide of claim 111, wherein the protein comprises an antibody or an antigen binding fragment of an antibody.

120. The polynucleotide of claim 119, comprising SEQ ID NO:15, a nucleotide sequence encoding SEQ ID NO: 16, SEQ ID NO:42, a nucleotide sequence encoding SEQ ID NO:43, SEQ ID NO:47, a nucleotide sequence encoding SEQ ID NO:48.

121. The polynucleotide of claim 111, which is operatively linked to a polynucleotide encoding a first ribosome binding sequence (RBS) and a second RBS, wherein the first RBS and second RBS are spaced apart by about 5 to 25 nucleotides, and wherein the first RBS directs translation of the fluorescent protein in a prokaryote and the second RBS directs translation of the fluorescent protein in a chloroplast.

122. A polypeptide encoded by the polynucleotide of claim 111.

123. A recombinant nucleic acid molecule, comprising
a first polynucleotide encoding at least one polypeptide, wherein codons of the first polynucleotide are biased to reflect chloroplast codon usage; and
a second polynucleotide, comprising a nucleotide sequence encoding a first ribosome binding sequence (RBS) operatively linked to a nucleotide sequence encoding a second RBS, wherein the first RBS can direct translation of the polypeptide in a prokaryote and the second RBS can direct translation of the polypeptide in a chloroplast.

124. The recombinant nucleic acid molecule of claim 123, wherein the first polynucleotide comprises a first nucleotide sequence encoding a first polypeptide followed by and operatively linked to a second nucleotide sequence encoding a second polypeptide.

125. The recombinant nucleic acid molecule of claim 124, wherein nucleotide sequence encoding an internal ribosome entry site is operatively linked to the second nucleotide sequence encoding the second polypeptide.

126. The recombinant nucleic acid molecule of claim 123, further comprising a third polynucleotide operatively linked to the first polynucleotide and the second polynucleotide.

127. The recombinant nucleic acid molecule of claim 126, wherein the third polynucleotide encodes at least one polypeptide.

128. A method of making a chloroplast/prokaryote shuttle expression vector, the method comprising introducing into a nucleotide sequence of chloroplast genomic deoxyribonucleic acid (DNA) sufficient to undergo homologous recombination with chloroplast genomic DNA
a nucleotide sequence comprising a prokaryote origin of replication,
a nucleotide sequence encoding a first ribosome binding sequence (RBS), and
a nucleotide sequence encoding a second RBS,
wherein the first RBS and second RBS are spaced apart by about 5 to 25 nucleotides, and
a cloning site, wherein the cloning site is positioned to allow operative linkage of a polynucleotide encoding a polypeptide to the first RBS and second RBS, whereby the first RBS can direct translation of the polypeptide in a prokaryote and the second RBS can direct translation of the polypeptide in a chloroplast.

129. A chloroplast/prokaryote shuttle expression vector produced by the method of claim 128.

130. A method of making a chloroplast/prokaryote shuttle expression vector, the method comprising genetically modifying a nucleotide sequence of chloroplast genomic deoxyribonucleic acid (DNA), which is sufficient to undergo homologous recombination with chloroplast genomic DNA, to contain
a prokaryote origin of replication,
a nucleotide sequence encoding a first ribosome binding sequence (RBS) spaced apart from a second RBS by about 5 to 25 nucleotides, and
a cloning site positioned to allow operative linkage of a polynucleotide encoding a polypeptide to the first RBS and second RBS, whereby the first RBS can direct translation of the polypeptide in a prokaryote and the second RBS can direct translation of the polypeptide in a chloroplast.

131. A chloroplast/prokaryote shuttle expression vector produced by the method of claim 130.

132. A recombinant polynucleotide, comprising a first nucleotide sequence encoding a chloroplast ribosome binding sequence (RBS) operatively linked to a second nucleotide sequence encoding a polypeptide, wherein the first nucleotide sequence is heterologous with respect to the second nucleotide sequence.

133. The recombinant polynucleotide of claim 132, wherein the chloroplast RBS is positioned 20 to 40 nucleotides 5′ to an initiator ATG codon, which is operatively linked to the nucleotide sequence encoding the polypeptide.

134. The recombinant polynucleotide of claim 132, wherein the first nucleotide sequence comprises an ATG codon positioned about 20 to 40 nucleotides 3′ of the RBS.

135. A vector, comprising a nucleotide sequence encoding a ribosome binding sequence (RBS) positioned about 20 to 40 nucleotides 5′ to a cloning site.

136. The vector of claim 135, wherein the cloning site comprises at least one restriction endonuclease recognition site or one recombinase recognition site, or a combination thereof.

137. The vector of claim 135, wherein the cloning site comprises a multiple cloning site consisting of a plurality of restriction endonuclease recognition sites or recombinase recognition sites, or a combination of at least one restriction endonuclease recognition site and at least one recombinase recognition site.

138. The vector of claim 134, further comprising an initiator ATG codon or a portion thereof adjacent and 5′ to the cloning site.

139. The vector of claim 135, further comprising a chloroplast gene 3′ untranslated region positioned 3′ to the cloning site.

140. A method of producing a polypeptide in a plastid, comprising introducing at least a first recombinant nucleic acid molecule into the plastid,
said first recombinant nucleic acid molecule comprising a first nucleotide sequence encoding at least one ribosome binding sequence (RBS) operatively linked to at least one heterologous polynucleotide encoding at least one polypeptide,
wherein the RBS directs translation of the polypeptide in a plastid,
under conditions that allow expression of the at least one polypeptide, thereby producing the polypeptide in the plastid.

141. The method of claim 140, wherein the plastid is a chloroplast.

142. The method of claim 141, wherein codons of the first polynucleotide are biased to reflect chloroplast codon usage.

143. The method of claim 140, wherein the first polynucleotide encodes an antibody, or a subunit of an antibody.

144. The method of claim 143, wherein the antibody specifically binds tetanus toxin or a herpes simplex virus.

145. The method of claim 140, wherein the first polynucleotide encodes a first polypeptide and, optionally, a second polypeptide.

146. The method of claim 145, wherein the first polynucleotide is biased for chloroplast codon usage.

147. The method of claim 146, wherein the first polypeptide comprises an immunoglobulin heavy chain or a variable region thereof, and the second polypeptide comprises an immunoglobulin light chain or a variable region thereof.

148. The method of claim 147, wherein the antibody comprises an amino acid sequence as set forth in SEQ ID NO:16, SEQ ID NO:43, or SEQ ID NO:48.

149. The method of claim 147, wherein the first polynucleotide comprises a nucleotide sequence as set forth in SEQ ID NO:15, SEQ ID NO:42, or SEQ ID NO:47.

150. The method of claim 146, wherein the heterologous polynucleotide encodes a reporter protein.

151. The method of claim 150, wherein the reporter protein comprises a green fluorescent protein or a luciferase.

152. The method of claim 151, wherein the heterologous polynucleotide comprises SEQ ID NO:1, a nucleotide sequence encoding SEQ ID NO:2, SEQ ID NO:45, or a nucleotide sequence encoding SEQ ID NO:46.

153. The method of claim 150, wherein the first polynucleotide encodes a first polypeptide and at least a second polypeptide.

154. The method of claim 153, wherein the first polypeptide and second polypeptide comprise subunits of a protein complex.

155. The method of claim 154, wherein the protein complex is a heterodimer.

156. The method of claim 150, further comprising introducing at least a second recombinant nucleic acid molecule into the plastid.

157. The method of claim 156, wherein the second recombinant nucleic acid molecule comprises a comprises a first nucleotide sequence encoding at least a first RBS operatively linked to at least a second heterologous polypeptide encoding at least a second polypeptide, wherein the first RBS can direct translation of the polypeptide in a chloroplast.

158. The method of claim 157, wherein the first recombinant nucleic acid molecule and the second recombinant nucleic acid molecule are co-expressed in the chloroplast.

159. The method of claim 140, wherein the first recombinant nucleic acid molecule is contained in a vector.

160. The method of claim 159, wherein the vector is a chloroplast vector, which comprises a nucleotide sequence of chloroplast genomic deoxyribonucleic acid (DNA) that can undergo homologous recombination with chloroplast genomic DNA.

161. The method of claim 160, wherein the vector further comprises a prokaryote origin of replication.

162. The method of claim 140, further comprising isolating the polypeptide from the plastid.

163. An isolated polypeptide obtained by the method of claim 162.

164. The isolated polypeptide of claim 163, which is an antibody or a reporter protein.

165. A synthetic polynucleotide, comprising at least a first nucleotide sequence encoding at least a first polypeptide, wherein at least one codon in the first nucleotide sequence is biased to reflect chloroplast codon usage.

166. The polynucleotide of claim 165, wherein each codon in the first nucleotide sequence is biased to reflect chloroplast codon usage.

167. The polynucleotide of claim 165, wherein the polynucleotide further comprises at least a second nucleotide sequence encoding a second polypeptide.

168. The polynucleotide of claim 167, wherein at least one codon of the second nucleotide sequence is biased to reflect chloroplast codon usage.

169. The polynucleotide of claim 167, wherein the first nucleotide sequence is operatively linked to the second nucleotide sequence.

170. The polynucleotide of claim 169, which encodes a fusion protein comprising the first polypeptide and the second polypeptide.

171. The polynucleotide of claim 169, wherein the first nucleotide sequence is operatively linked to the second nucleotide sequence via a third nucleotide sequence.

172. The polynucleotide of claim 171, wherein the third nucleotide sequence encodes a linker peptide.

173. The polynucleotide of claim 172, which encodes a fusion protein comprising the first polypeptide linked via the linker peptide to the second polypeptide.

174. The polynucleotide of claim 165, wherein the first polypeptide comprises an immunoglobulin variable region, an immunoglobulin constant region, or a combination thereof.

175. The polynucleotide of claim 167, which encodes a single chain antibody comprising a heavy chain variable region operatively linked to a light chain variable region.

176. The polynucleotide of claim 175, wherein the single chain antibody has an amino acid sequence as set forth in SEQ ID NO:16, SEQ ID NO:43, or SEQ ID NO:48.

177. The polynucleotide of claim 175, which has a nucleotide sequence as set forth in SEQ ID NO:15, SEQ ID NO:42, or SEQ ID NO:47.

178. The polynucleotide of claim 165, which encodes a reporter polypeptide.

179. The polynucleotide of claim 178, wherein the reporter polypeptide is a luciferase.

180. The polynucleotide of claim 179, wherein the luciferase has an amino acid sequence as set forth in SEQ ID NO:46.

181. The polynucleotide of claim 180, which has a nucleotide sequence as set forth in SEQ ID NO:45.

182. A polypeptide, comprising an amino acid sequence as set forth in SEQ ID NO:16, SEQ ID NO:43, SEQ ID NO:46, or SEQ ID NO:48.

183. A method of producing a heterologous polypeptide in a plastid, the method comprising introducing the synthetic polynucleotide of claim 165 into the plastid under conditions that allow expression of the at least first polypeptide in the plastid.

184. The method of claim 183, wherein the synthetic polynucleotide is operatively linked to a nucleic acid sequence encoding at least one ribosome binding sequence (RBS).

185. The method of claim 184, wherein the RBS can direct translation of the polypeptide in a plastid.

186. The method of claim 184, wherein the polynucleotide further comprises at least a second nucleotide sequence encoding a second polypeptide.

187. The method of claim 186, wherein the first nucleotide sequence is operatively linked to the second nucleotide sequence.

188. The method of claim 187, wherein the heterologous polypeptide comprises a fusion protein comprising the first polypeptide and the second polypeptide.

189. The method of claim 187, wherein the first nucleotide sequence is operatively linked to the second nucleotide sequence via a third nucleotide sequence.

190. The method of claim 189, wherein the third nucleotide sequence encodes a linker peptide.

191. The method of claim 190, wherein the heterologous polypeptide comprises a fusion protein comprising the first polypeptide linked via the linker peptide to the second polypeptide.

192. The method of claim 183, wherein the heterologous polypeptide comprises an immunoglobulin variable region, an immunoglobulin constant region, or a combination thereof.

193. The method of claim 183, wherein the heterologous polypeptide comprises a single chain antibody comprising a heavy chain variable region operatively linked to a light chain variable region.

194. The method of claim 193, wherein the single chain antibody has an amino acid sequence as set forth in SEQ ID NO:16, SEQ ID NO:43, or SEQ ID NO:48.

195. The method of claim 193, wherein the single chain antibody is encoded by a nucleotide sequence as set forth in SEQ ID NO:15, SEQ ID NO:42, or SEQ ID NO:47.

196. The method of claim 183, wherein the heterologous polypeptide comprises a reporter polypeptide.

197. The method of claim 196, wherein the reporter polypeptide is a luciferase.

198. The method of claim 197, wherein the luciferase has an amino acid sequence as set forth in SEQ ID NO:46.

199. The method of claim 198, wherein the reporter polypeptide is encoded by a nucleotide sequence as set forth in SEQ ID NO:45.

200. The method of claim 183, wherein the plastid is a chloroplast.

201. The method of claim 200, wherein the chloroplast is in an algae.

202. The method of claim 201, wherein the algae is a microalgae.

203. A heterologous polypeptide produced by the method of claim 183.

204. A method of detecting a plant cell, comprising introducing the polynucleotide of claim 178 into a chloroplast of the plant cell under conditions that allow expression of the reporter polypeptide in the chloroplast, and detecting expression of the reporter polypeptide.

205. The method of claim 204, wherein the reporter polypeptide is a luciferase.

206. The method of claim 205, wherein the luciferase has an amino acid sequence as set forth in SEQ ID NO:46.

207. The method of claim 204, wherein the polynucleotide has nucleotide sequence as set forth in SEQ ID NO:45.


SEQUENCE LISTING (48 sequences)

SEQ ID NO: 1 - DNA, 717 bp, Artificial sequence
  Chloroplast codon optimized Green Fluorescent Protein
atggctaaaggtgaagaattattcacaggtgttgtacctattttagtagaattagacggtgatgtaaacggtcacaaattttcagtttctggtgaaggtgaaggtgacgcaacttatggtaaattaacacttaaattcatttgtactacaggtaaattaccagtaccttggccaactttagttacaacttttacatacggtgtacaatgtttcagtcgttaccctgatcacatgaaacaacatgactttttcaaatctgctatgccagaaggttatgttcaagaacgtactatttttttcaaagatgacggtaattataaaacacgtgctgaagtaaaatttgaaggtgatactttagttaaccgtattgaattaaaaggtattgacttcaaagaagatggtaatattttaggtcacaaacttgaatataactacaattcacataacgtatatattatggcagacaaacaaaaaaatggtattaaagtaaactttaaaattcgtcataatatcgaggatggttctgtacaattagctgaccactatcaacaaaacacaccaattggtgatggtcctgttttacttccagacaatcattatttaagtactcaatctgctttatcaaaagatcctaacgaaaaacgtgaccacatggtattacttgaatttgttacagcagctggtattactcacggtatggatgaattatacaaataa

SEQ ID NO: 2 - protein, 238 aa, Artificial sequence
  Chloroplast codon optimized Green Fluorescent Protein
MAKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - DNA, 717 bp, Aequorea victoria
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 4 - DNA, 544 bp, Chlamydomonas reinhardtii
ggatcccatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcatatg

SEQ ID NO: 5 - DNA, 241 bp, Chlamydomonas reinhardtii
ggatccactagtaacggccgccagtgtgctggaattcggcttccgaattcatatacctaaaggccctttctatgctcgactgataagacaagtacataaatttgctagtttacattattttttatttctaaatatataatatatttaaatgtatttaaaatttttcaacaatttttaaattatatttccggacagattattttaggatcgtcaaaagaagttacatttatttatacatatg

SEQ ID NO: 6 - DNA, 468 bp, Chlamydomonas reinhardtii
ggatccctagtaacggccgccagtgtgctggaatttgagtatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcatatg

SEQ ID NO: 7 - DNA, 373 bp, Chlamydomonas reinhardtii
ggatccgtcgactggtaccgccactgcctgcttcctccttcggagtatgtaaaccccttcgggcaactaaagtttatcgcagtatataaatataggcagttggcaggcaactgccactgacgtcctattttaatactccgaaggaggcagttggcaggcaactgccactgacgtcccgtaagggtaaggggacgtccactggcgtcccgtaaggggaaggggacgtaggtacataaatgtgctaggtaactaacgtttgattttttgtggtataatatatgtaccatgcttttaatagaagcttgaatttataaattaaaatatttttacaatattttacgagaaattaaaactttaaaaaaattaacatatg

SEQ ID NO: 8 - DNA, 223 bp, Chlamydomonas reinhardtii
ggatccgttggcaggcaacaaatttatttattgtcccgtaaggggaagggggaaacaattattattttactgcggagcagcttgttattgaagttttattaaaaaaaaaataaaaatttgacaaaaaaaataaaaaagttaaattaaaaacactgggaatgttctacatcataaaaatcaaaagggtttaaaatcccgacaaaatttaaactttaaacatatg

SEQ ID NO: 9 - DNA, 397 bp, Chlamydomonas reinhardtii
tctagacttagcttcaactaactctagctcaaacaactaatttttttttaaactaaaataaatctggttaaccatacctggtttattttagtttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgacggatcc

SEQ ID NO: 10 - DNA, 434 bp, Chlamydomonas reinhardtii
ctagagtcgacctgcaggcatgcaagcttgtactcaagctcggaacgaaggtcgtgccttgctcggaaggtggcgacgtaattcgttcagcttgtaaatggtctcccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatacgtcgacggatcc

SEQ ID NO: 11 - DNA, 411 bp, Chlamydomonas reinhardtii
tctagattttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacagggatcc

SEQ ID NO: 12 - DNA, 266 bp, Chlamydomonas reinhardtii
tctagattataatacattaaaattgtaacgcctttacaagacagtataaaatgggaattaattaattaggagggtcactttcagccactcgttttttaaataggtaagtaacctttttaagagaacgtaagagattgtggattacgttctcaagagacataactcaaaatactagtaggtttgagcttgacttcaagctttaacctccgtcagcgataaaacctattttgagcgcattttaatatatttgggacgccagtggatcc

SEQ ID NO: 13 - DNA, 792 bp, Artificial sequence
  Chloroplast codon optimized antibody specific for tetanus toxin
atgctcgagcagtctggggctgaggtgaagaagcctgggtcctcggtgaaggtctcctgcagggcttctggaggcaccttcaacaattatgccatcagctgggtgcgacaggcccctggacaagggcttgagtggatgggagggatcttccctttccgtaatacagcaaagtacgcacaacacttccagggcagggtcaccattaccgcggacgaatccacgggcacagcctacatggagctgagcagcctgagatctgaggacacggccatatattattgtgcgagaggggatacgatttttggagtgaccatgggatactacgctatggacgtctggggccaagggaccaccgtcaccgtctcctctggtggcggtggctcgggcggtggtgggtcgggtggcggcggatctgagctcgttctcacgcagtctccaggcaccctgtctttgtctccaggggaaagagccaccctctcctgcagggccagtcacagtgttagcagggcctacttagcctggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtactactgtcagcagtatggtggctcaccgtggttcggccaagggaccaaggtggaactcaaacgaactagtggccaggccggccagtacccgtacgacgttccggactacgcttcttaa

SEQ ID NO: 14 - protein, 263 aa, Artificial sequence
  Chloroplast codon optimized antibody specific for tetanus toxin
MLEQSGAEVKKPGSSVKVSCRASGGTFNNYAISWVRQAPGQGLEWMGGIFPFRNTAKYAQHFQGRVTITADESTGTAYMELSSLRSEDTAIYYCARGDTIFGVTMGYYAMDVWGQGTTVTVSSGGGGSGGGGSGGGGSELVLTQSPGTLSLSPGERATLSCRASHSVSRAYLAWYQQKPGQAPRLLIYGTSSRATGIPDRFSGSGSGTDFTLTISRLEPEDFAVYYCQQYGGSPWFGQGTKVELKRTSGQAGQYPYDVPDYAS

SEQ ID NO: 15 - DNA, 1926 bp, Artificial sequence
  Chloroplast codon optimized antibody specific for Herpes simplex virus
catatggctgctcaccaccaccaccaccacgttgctcaagctgcttcatcagaattaacgcaatcaccaggtaccttatcattatcaccaggtgaacgtgctaccttatcatgtcgtgcttcacaatcagtttcatcagcttacttagcttggtaccaacaaaaaccaggtcaagctccacgtttattaatttacggtgcttcatcacgtgctactggtattccagatcgtttctcaggttcaggttcaggtacagatttcactttaaccatttcacgtttagaaccagaagatttcgctgtttactactgtcaacaatacggtcgttcaccaactttcggtggtggtaccaaagttgaaattaaacgtacttcatcaggtggtggtggttcaggtggtggtggtggtggttcatcacgttcatcattagaacaatcaggtgctgaagttaaaaaaccaggttcatcagttaaagtttcatgtaaagcttcaggtggttcattctcatcatacgctattaactgggttcgtcaagctcaaggtcaaggtttagaatggatgggtggtttaatgccaattttcggtacaacaaactacgctcaaaaattccaagatcgtttaacgattaccgctgatgtttcaacgtcaacagcttacatgcaattatcaggtttaacatacgaagatacggctatgtactactgtgctcgtgttgcttacatgttagaaccaaccgttactgctggtggtttagatgtttggggtaaaggtaccacggttaccgtttcaccagcttcaccaacctcaccaaaagttttcccattatcattatgttcaacccaaccagatggtaacgttgttattgcttgtttagttcaaggtttcttcccacaagaaccattatcagttacctggtcagaatcaggtcaaggtgttaccgctcgtaacttcccaccatcacaagatgcttcaggtgatttatacaccacgtcatcacaattaaccttaccagctacacaatgtttagctggtaaatcagttacatgtcacgttaaacactacacgaacccatcacaagatgttactgttccatgtccagttccatcaactccaccaaccccatcaccatcaactccaccaaccccatcaccatcatgttgtcacccacgtttatcattacaccgtccagctttagaagatttattattaggttcagaagctaacttaacgtgtacattaaccggtttacgtgatgcttcaggtgttaccttcacctggacgccatcatcaggtaaatcagctgttcaaggtccaccagaacgtgatttatgtggttgttactcagtttcatcagttttaccaggttgtgctgaaccatggaaccacggtaaaaccttcacttgtactgctgcttacccagaatcaaaaaccccattaaccgctaccttatcaaaatcaggtaacacattccgtccagaagttcacttattaccaccaccatcagaagaattagctttaaacgaattagttacgttaacgtgtttagctcgtggtttctcaccaaaagatgttttagttcgttggttacaaggttcacaagaattaccacgtgaaaaatacttaacttgggcttcacgtcaagaaccatcacaaggtaccaccaccttcgctgttacctcaattttacgtgttgctgctgaagattggaaaaaaggtgataccttctcatgtatggttggtcacgaagctttaccattagctttcacacaaaaaaccattgatcgtttagctggtaaaccaacccacgttaacgtttcagttgttatggctgaagttgatggtacctgttacgattataaagatcacgatggtgattacaaagatcacgatattgattataaagatgatgatgataaataatctaga

SEQ ID NO: 16 - protein, 639 aa, Artificial sequence [sequence omitted]

SEQ ID NO: 17 - DNA, 22 bp, Artificial sequence
  Amplification primer
catatgagtaaaggagaagaac

SEQ ID NO: 18 - DNA, 25 bp, Artificial sequence
  Amplification primer
tctagattatttgtatagttcatcc

SEQ ID NO: 19 - DNA, 18 bp, Artificial sequence
  Amplification primer
tctagagtcgacctgcag

SEQ ID NO: 20 - DNA, 17 bp, Artificial sequence
  Amplification primer
ggatccgtcgacgtatg

SEQ ID NO: 21 - DNA, 31 bp, Artificial sequence
  Amplification primer
gaattcatatacctaaaggccctttctatgc

SEQ ID NO: 22 - DNA, 25 bp, Artificial sequence
  Amplification primer
catatgtataaataaatgtaacttc

SEQ ID NO: 23 - DNA, 57 bp, Artificial sequence
  Amplification primer
gaagcttgaatttataaattaaaatatttttacaatattttacccagaaattaaaac

SEQ ID NO: 24 - DNA, 44 bp, Artificial sequence
  Amplification primer
tgtcatatgttaatttttttaaagtttttctccgtaaaatattg

SEQ ID NO: 25 - DNA, 40 bp, Artificial sequence
  Amplification primer
tgtcatatgttaatttttttaaagtctccgtaaaatattg

SEQ ID NO: 26 - DNA, 36 bp, Artificial sequence
  Amplification primer
tgtcatatgttaattttttttctccgtaaaatattg

SEQ ID NO: 27 - DNA, 20 bp, Artificial sequence
  Amplification primer
gtcatatgttaatttctccg

SEQ ID NO: 28 - DNA, 38 bp, Artificial sequence
  Amplification primer
tgtcatatgttaatcctcctaaagttttaatttctccg

SEQ ID NO: 29 - RNA, 10 nt, Chlamydomonas reinhardtii
caccuccuuc

SEQ ID NO: 30 - DNA, 2000 bp, Chlamydomonas reinhardtii
  CDS (497)..(1552)
cgtcatagtatatcaatattgtaacagattgacaccctttaagtaaacattttttttgagtcatatggagtcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcattaggtggttggttaactggtactactttcgttacttcatggtatacgcatggtttagctacttcttacttagaaggttgtaacttcttaacagcagctgtttctacacctgctaacagtatggctcactctcttctatttgtttggggtccagaagctcaaggtgatttcactcgttggtgtcaacttggtggtttatgggcattcgttgctttacacggtgcatttggtttaattggtttcatgcttcgtcagtttgaaattgctcgttcagtaaacttacgtccatacaacgcaattgctttctcagcaccaattgctgtattcgtttcagtattcctaatttacccattaggtcaatcaggttggttctttgcacctagtttcggtgtagctgctatcttccgtttcattttattcttccaaggtttccacaactggacacttaacccattccacatgatgggtgttgctggtgttttaggtgctgctttattatgtgctattcacggtgctactgttgaaaacacattattcgaagacggtgacggtgctaacacattccgtgcattcaaccctacacaggctgaagaaacatactctatggttactgctaaccgtttctggtcacaaatcttcggtgttgctttctctaacaaacgttggcttcacttcttcatgttattagttccagtaactggtctttggatgagtgctattggtgttgtaggtttagctctaaacttacgtgcttacgacttcgtatcacaagagattcgtgctgctgaagaccctgaattcgaaacattctacactaaaaacattcttcttaacgaaggtattcgtgcttggatggctgctcaagaccaaccacacgaacgtttagtattccctgaagaagtattaccacgtggtaacgctctataatatatttttatataaattaccaatactaattagtattggtaatttatattactttattatttaaaagaaaatgcccctttggggctaaaaatcacatgagtgcttgagccgtatgcgaaaaaactcgcatgtacggttctttaggaggatttaaaatattaaaaaataaaaaaacaaatcctacctgactaaaccaggacatttttcacgtactctgtcaaaaggtccaaacacaacaacttggatttggaaccttcacgcagatgctcatgactttgacagtcatacaagtgatctagaagaaatttctagaaaagtattcagtgcacactttggtcaattaggtatcattttcatttggttaagtgggtgcgacacgaagacgtatatatttttatagtttaaaaagatacttttacactgtagttgaaaagtataagcactt

SEQ ID NO: 31 - protein, 352 aa, Chlamydomonas reinhardtii
MTIAIGTYQEKRTWFDDADDWLRQDRFVFVGWSGLLLFPCAYFALGGWLTGTTFVTSWYTHGLATSYLEGCNFLTAAVSTPANSMAHSLLFVWGPEAQGDFTRWCQLGGLWAFVALHGAFGLIGFMLRQFEIARSVNLRPYNAIAFSAPIAVFVSVFLIYPLGQSGWFFAPSFGVAAIFRFILFFQGFHNWTLNPFHMMGVAGVLGAALLCAIHGATVENTLFEDGDGANTFRAFNPTQAEETYSMVTANRFWSQIFGVAFSNKRWLHFFMLLVPVTGLWMSAIGVVGLALNLRAYDFVSQEIRAAEDPEFETFYTKNILLNEGIRAWMAAQDQPHERLVFPEEVLPRGNAL

SEQ ID NO: 32 - RNA, 45 nt, Chlamydomonas reinhardtii
caauauuuuacggagaaauuaaaacuuuaaaaaaauuaacauaug

SEQ ID NO: 33 - RNA, 13 nt, Chlamydomonas reinhardtii
gcucaccuccuuc

SEQ ID NO: 34 - RNA, 38 nt, Chlamydomonas reinhardtii
uuacggagaaauuaaaacuuuaaaaaaauuaacauaug

SEQ ID NO: 35 - RNA, 34 nt, Artificial sequence
  Mutant sequence of SEQ ID NO32
uuacaaauuaaaacuuuaaaaaaauuaacauaug

SEQ ID NO: 36 - RNA, 38 nt, Artificial sequence
  Mutant sequence of SEQ ID NO32
uuacccagaaauuaaaacuuuaaaaaaauuaacauaug

SEQ ID NO: 37 - RNA, 34 nt, Artificial sequence
  Mutant sequence of SEQ ID NO32
uuacggagaaaaacuuuaaaaaaauuaacauaug

SEQ ID NO: 38 - RNA, 30 nt, Artificial sequence
  Mutant sequence of SEQ ID NO32
uuacggagacuuuaaaaaaauuaacauaug

SEQ ID NO: 39 - RNA, 26 nt, Artificial sequence
  Mutant sequence of SEQ ID NO32
uuacggagaaaaaaaauuaacauaug

SEQ ID NO: 40 - RNA, 22 nt, Artificial sequence
  Mutant sequence of SEQ ID NO32
uuacggagaaauuaaacauaug

SEQ ID NO: 41 - RNA, 38 nt, Artificial sequence
  Mutant sequence of SEQ ID NO32
uuacggagaaauuaaaacuuuaggaggauuaacauaug

SEQ ID NO: 42 - DNA, 840 bp, Homo sapiens
catatggttgctcaagctgcttcatcagaattaacgcaatcaccaggtaccttatcattatcaccaggtgaacgtgctaccttatcatgtcgtgcttcacaatcagtttcatcagcttacttagcttggtaccaacaaaaaccaggtcaagctccacgtttattaatttacggtgcttcatcacgtgctactggtattccagatcgtttctcaggttcaggttcaggtacagatttcactttaaccatttcacgtttagaaccagaagatttcgctgtttactactgtcaacaatacggtcgttcaccaactttcggtggtggtaccaaagttgaaattaaacgtacttcatcaggtggtggtggttcaggtggtggtggtggtggttcatcacgttcatcattagaacaatcaggtgctgaagttaaaaaaccaggttcatcagttaaagtttcatgtaaagcttcaggtggttcattctcatcatacgctattaactgggttcgtcaagctcaaggtcaaggtttagaatggatgggtggtttaatgccaattttcggtacaacaaactacgctcaaaaattccaagatcgtttaacgattaccgctgatgtttcaacgtcaacagcttacatgcaattatcaggtttaacatacgaagatacggctatgtactactgtgctcgtgttgcttacatgttagaaccaaccgttactgctggtggtttagatgtttggggtaaaggtaccacggttaccgtttcagattataaagatcacgatggtgattacaaagatcacgatattgattataaagatgatgatgataaataatctaga

SEQ ID NO: 43 - protein, 277 aa, Homo sapiens [sequence omitted]

SEQ ID NO: 44 - protein, 681 aa, Vibrio harveyi
MKFGNFLLTYQPPELSQTEVMKRLVNLGKASEGCGFDTVWLLEHHFTEFGLLGNPYVAAAHLLGTTETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQZMKFGLFFLNFMNSKRSSDQVIEEILDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVNANIVKYHSZ

SEQ ID NO: 45 - DNA, 2094 bp, Artificial sequence
  Chloroplast codon biased luxAB gene
catatgaaatttggtaacttccttttaacttatcaaccacctgaactatctcaaacagaagttatgaaacgtttagttaatttaggtaaagcttctgaaggttgtggtttcgacacagtttggttattagaacatcactttactgaatttggtttattaggtaacccttatgttgctgctgcacatctattaggtgctacagaaaaattaaatgttggtactgctgctattgtattacctactgctcaccctgttcgtcaagcagaagacgtaaatttattagatcaaatgtcaaaaggacgttttcgttttggtatttgtcgtggtttatacgacaaagatttccgtgtttttggtacagacatggataatagtcgtgctttaatggactgttggtatgacttaatgaaagaaggttttaacgaaggttatattgctgcagataatgaacatattaaattccctaaaattcaattaaatccatcagcttacacacaaggtggtgctcctgtttatgttgttgctgaatcagcatcaacaacagaatgggctgctgaacgtggtttaccaatgattctaagttggattattaatactcacgaaaaaaaagcacaacttgatctttataatgaagttgctactgaacacggttacgatgtaactaaaattgaccattgtttatcttatattacttcagttgatcacgattcaaacaaagctaaagatatttgtcgtaattttttaggtcattggtatgactcatacgtaaatgctacaaaaatttttgatgactctgatcaaacaaaaggttatgactttaataaaggtcaatggcgtgattttgttttaaaaggtcacaaagatactaaccgtcgtattgattatagttacgaaattaatccagtaggtacacctgaagaatgtatcgcaattattcaacaagatatcgatgctacaggtattaataatatttgttgtggttttgaagctaacggttctgaagaagaaattatcgcttctatgaaattatttcaatctgatgtaatgccatatcttaaagaaaaacaatctggtggtggaggttcttcaggtggtggaggcggtggttcttcaatgaaatttggattatttttccttaattttatgaattcaaaacgttcttctgatcaagttattgaagaaatgttagatactgcacattatgtagatcaattaaaatttgacacattagctgtttacgaaaatcacttttcaaacaatggtgtagttggtgctccattaacagtagctggttttttacttggtatgacaaaaaacgctaaagtagcttcattaaatcatgttattactacacaccatccagtacgtgtagctgaagaagcatgtttacttgatcaaatgagtgaaggtcgttttgtttttggttttagtgattgtgaaaaaagtgctgatatgcgtttttttaatcgtccaacagattctcaatttcaattattcagtgaatgtcacaaaattatcaatgatgcatttactactggttattgtcatccaaataatgatttttacagttttcctaaaatttctgttaacccacacgcttatactgaaggtggtcctgcacaatttgtaaatgctacaagtaaagaagtagttgaatgggcagctaaattaggtcttccacttgtatttaaatgggacgattcaaatgctcaacgtaaagaatatgctggtttataccatgaagttgctcaagcacacggtgttgatgttagtcaagttcgtcataaattaacactattagttaatcaaaacgtagatggtgaagcagctcgtgcagaagctcgtgtttatttagaagaatttgttcgtgaatcttatcctaatactgacttcgaacaaaaaatggtagaattattatcagaaaacgctattggtacttacgaagaaagtactcaagcagctcgtgttgcaattgaatgttgtggtgctgcagacttattaatgtcttttgaatcaatggaagataaagctcacgaacgtgcagttattgatgtagtaaatgctaacattgttaaatatcattcataatctaga

SEQ ID NO: 46 - protein, 698 aa, Artificial sequence
  LuxAB fusion protein
HMKFGNFLLTYQPPELSQTEVMKRLVNLGKASEGCGFDTVWLLEHHFTEFGLLGNPYVAAAHLLGATEKLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNKAKDICRNFLGHWYDSYVNATKIFDDSDQTKGYDFNKGQWRDFVLKGHKDTNRRIDYSYEINPVGTPEECIAIIQQDIDATGINNICCGFEANGSEEEIIASMKLFQSDVMPYLKEKQSGGGGSSGGGGGGSSMKFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFVFGFSDCEKSADMRFFNRPTDSQFQLFSECHKIINDAFTTGYCHPNNDFYSFPKISVNPHAYTEGGPAQFVNATSKEVVEWAAKLGLPLVFKWDDSNAQRKEYAGLYHEVAQAHGVDVSQVRHKLTLLVNQNVDGEAARAEARVYLEEFVRESYPNTDFEQKMVELLSENAIGTYEESTQAARVAIECCGAADLLMSFESMEDKAHERAVIDVVNANIVKYHSZSR

SEQ ID NO: 47 - DNA, 1893 bp, Artificial sequence
  Single-chain antibody
atggttgctcaagctgcttcatcagaattaacgcaatcaccaggtaccttatcattatcaccaggtgaacgtgctaccttatcatgtcgtgcttcacaatcagtttcatcagcttacttagcttggtaccaacaaaaaccaggtcaagctccacgtttattaatttacggtgcttcatcacgtgctactggtattccagatcgtttctcaggttcaggttcaggtacagatttcactttaaccatttcacgtttagaaccagaagatttcgctgtttactactgtcaacaatacggtcgttcaccaactttcggtggtggtaccaaagttgaaattaaacgtacttcatcaggtggtggtggttcaggtggtggtggtggtggttcatcacgttcatcattagaacaatcaggtgctgaagttaaaaaaccaggttcatcagttaaagtttcatgtaaagcttcaggtggttcattctcatcatacgctattaactgggttcgtcaagctcaaggtcaaggtttagaatggatgggtggtttaatgccaattttcggtacaacaaactacgctcaaaaattccaagatcgtttaacgattaccgctgatgtttcaacgtcaacagcttacatgcaattatcaggtttaacatacgaagatacggctatgtactactgtgctcgtgttgcttacatgttagaaccaaccgttactgctggtggtttagatgtttggggtaaaggtaccacggttaccgtttcaccagcttcaccaacctcaccaaaagttttcccattatcattatgttcaacccaaccagatggtaacgttgttattgcttgtttagttcaaggtttcttcccacaagaaccattatcagttacctggtcagaatcaggtcaaggtgttaccgctcgtaacttcccaccatcacaagatgcttcaggtgatttatacaccacgtcatcacaattaaccttaccagctacacaatgtttagctggtaaatcagttacatgtcacgttaaacactacacgaacccatcacaagatgttactgttccatgtccagttccatcaactccaccaaccccatcaccatcaactccaccaaccccatcaccatcatgttgtcacccacgtttatcattacaccgtccagctttagaagatttattattaggttcagaagctaacttaacgtgtacattaaccggtttacgtgatgcttcaggtgttaccttcacctggacgccatcatcaggtaaatcagctgttcaaggtccaccagaacgtgatttatgtggttgttactcagtttcatcagttttaccaggttgtgctgaaccatggaaccacggtaaaaccttcacttgtactgctgcttacccagaatcaaaaaccccattaaccgctaccttatcaaaatcaggtaacacattccgtccagaagttcacttattaccaccaccatcagaagaattagctttaaacgaattagttacgttaacgtgtttagctcgtggtttctcaccaaaagatgttttagttcgttggttacaaggttcacaagaattaccacgtgaaaaatacttaacttgggcttcacgtcaagaaccatcacaaggtaccaccaccttcgctgttacctcaattttacgtgttgctgctgaagattggaaaaaaggtgataccttctcatgtatggttggtcacgaagctttaccattagctttcacacaaaaaaccattgatcgtttagctggtaaaccaacccacgttaacgtttcagttgttatggctgaagttgatggtacctgttacgattataaagatcacgatggtgattacaaagatcacgatattgattataaagatgatgatgataaataa

SEQ ID NO: 48 - protein, 630 aa, Artificial sequence
  Single-chain atibody
MVAQAASSELTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKRTSSGGGGSGGGGGGSSRSSLEQSGAEVKKPGSSVKVSCKASGGSFSSYAINWVRQAQGQGLEWMGGLMPIFGTTNYAQKFQDRLTITADVSTSTAYMQLSGLTYEDTAMYYCARVAYMLEPTVTAGGLDVWGKGTTVTVSPASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCYDYKDHDGDYKDHDIDYKDDDDK